(19) United States
(12) Patent Application Publication
(10) Pub. No.: US 2023/0374538 A1
(43) Pub. Date: Nov. 23, 2023

(54) Methods and Compositions for Expression of Nucleic Acids in Cells

(71) Applicant: Myeloid Therapeutics, Inc., Cambridge, MA (US)

(72) Inventors: Daniel GETTS, Stow, MA (US); Yuxiao WANG, Belmont, MA (US); Namita BISARIA, Somerville, MA (US); Inna SHCHERBAKOVA, Holliston, MA (US); William F. ROWLEY, JR., Jamaica Plain, MA (US)

(21) Appl. No.: 18/188,027

(22) Filed: Mar. 22, 2023

Related U.S. Application Data
(63) Continuation of application No. PCT/US2021/051539, filed on Sep. 22, 2021.
(60) Provisional application No. 63/082,388, filed on Sep. 23, 2020.

(51) Int. Cl. C12N 15/85; A61K 39/00; A61K 48/00

(52) U.S. Cl. CPC C12N 15/85; A61K 39/4614; A61K 39/4631; A61K 48/0066; C12N 2830/50

(57) ABSTRACT

Compositions and methods for making and using engineered phagocytic cells that express a chimeric antigen receptor having an enhanced phagocytic activity for stable and durable expression are described and can be suitably used for immunotherapy in cancer or infection.

### CROSS REFERENCE

This application is a continuation of international application PCT/US2021/051539 filed on Sep. 22, 2021, which claims the benefit of U.S. Provisional Application No. 63/082,388, filed on Sep. 23, 2020, which is incorporated herein by reference in its entirety.

### SEQUENCE LISTING

The instant application contains a Sequence Listing which has been submitted electronically in XML format and is hereby incorporated by reference in its entirety. Said XML copy named 56371-714_601.XML.xml, created on Mar. 21, 2023, is converted into ST.26 version in XML format from the Sequence Listing file created under ST.25 version in ASCII format as filed in the corresponding international application PCT/US2021/051539, and the XML file is 102,301 bytes in size.

### BACKGROUND

Cellular immunotherapy is a promising new technology for fighting difficult to treat diseases, such as cancer, and persistent infections and also certain diseases that are refractory to other forms of treatment. A major breakthrough has come across with the discovery of CAR-T cell and their potential use in immunotherapy. CAR-T cells are T lymphocytes expressing a chimeric antigen receptor which helps target the T cell to specific diseased cells such as cancer cells, and can induce cytotoxic responses intended to kill the target cancer cell or immunosuppression and/or tolerance depending on the intracellular domain employed and co-expressed immunosuppressive cytokines. However, several limitations along the way has slowed the progress on CAR-T cells and dampened its promise in clinical trials.

Understanding the limitations of CAR-T cells is the key to leveraging the technology and continue innovations towards better immunotherapy models. Specifically, in T cell malignancies, CAR-T cells appear to have faced a major problem. CAR-T cells and malignant T cells share surface antigen in most T cell lymphomas (TCL), therefore, CAR-T cells are subject to cytotoxicity in the same way as cancer cells. In some instances, the CAR-T products may be contaminated by malignant T cells. Additionally, T cell aplasia is a potential problem due to prolonged persistence of the CAR-T cells. Other limitations include the poor ability for CAR-T cells to penetrate into solid tumors and the potent tumor microenvironment which acts to downregulate their anti-tumor potential. CAR-T cell function is also negatively influenced by the immunosuppressive tumor microenvironment (TME) that leads to endogenous T cell inactivation and exhaustion.

Myeloid cells, including macrophages, are cells derived from the myeloid lineage and belong to the innate immune system. They are derived from bone marrow stem cells which egress into the blood and can migrate into tissues. Some of their main functions include phagocytosis, the activation of T cell responses, and clearance of cellular debris and extracellular matrices. They also play an important role in maintaining homeostasis, and initiating and resolving inflammation. Moreover, myeloid cells can differentiate into numerous downstream cells, including macrophages, which can display different responses ranging from pro-inflammatory to anti-inflammatory depending on the type of stimuli they receive from the surrounding microenvironment. Furthermore, tissue macrophages have been shown to play a broad regulatory and activating role on other immune cell types including CDT effector cells, NK cells and T regulatory cells. Macrophages have been shown to be a main immune infiltrate in malignant tumors and have been shown to have a broad immunosuppressive influence on effector immune infiltration and function.

Myeloid cells are a major cellular compartment of the immune system comprising monocytes, dendritic cells, tissue macrophages, and granulocytes. Models of cellular ontogeny, activation, differentiation, and tissue-specific functions of myeloid cells have been revisited during the last years with surprising results. However, their enormous plasticity and heterogeneity, during both homeostasis and disease, are far from understood. Although myeloid cells have many functions, including phagocytosis and their ability to activate T cells, harnessing these functions for therapeutic uses has remained elusive. Newer avenues are therefore sought for using other cell types towards development of improved therapeutics, including but not limited to T cell malignancies.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery.

### SUMMARY

The diverse functionality of myeloid cells makes them an ideal cell therapy candidate that can be engineered to have numerous therapeutic effects. The present disclosure is related to immunotherapy using myeloid cells (e.g., CD14+ cells) of the immune system, particularly phagocytic cells. A number of therapeutic indications could be contemplated using myeloid cells. For example, myeloid cell immunotherapy could be exceedingly important in cancer, autoimmunity, fibrotic diseases and infections. The present disclosure is related to immunotherapy using myeloid cells, including phagocytic cells of the immune system, particularly macrophages. It is an object of the invention disclosed herein to harness one or more of these functions of myeloid cells for therapeutic uses. For example, it is an object of the invention disclosed herein to harness the phagocytic activity of myeloid cells, including engineered myeloid cells, for therapeutic uses. For example, it is an object of the invention disclosed herein to harness the ability of myeloid cells, including engineered myeloid cells, to promote T cell activation. For example, it is an object of the invention disclosed herein to harness the ability of myeloid cells, including engineered myeloid cells, to promote secretion of tumoricidal molecules. For example, it is an object of the invention disclosed herein to harness the ability of myeloid cells, including engineered myeloid cells, to promote recruitment and trafficking of immune cells and molecules. The present disclosure provides innovative methods and compositions that can successfully electroporate, transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity.

The present disclosure involves making and using engineered myeloid cells (e.g., CD14+ cells, such as macrophages or other phagocytic cells, which can attack and kill (ATAK) diseased cells directly and/or indirectly, such as cancer cells and infected cells. Engineered myeloid cells, such as monocytes and macrophages and other phagocytic cells, can be prepared by incorporating nucleic acid sequences (e.g., mRNA, plasmids, viral constructs) encoding a gene of interest, such as a chimeric fusion protein (CFP), that has an extracellular binding domain specific to disease associated antigens (e.g., cancer antigens), into the cells using, for example, nucleic acid technology, synthetic nucleic acids, gene editing techniques (e.g., CRISPR), transduction (e.g., using viral constructs), electroporation, or nucleofection. It has been found that myeloid cells can be engineered to have a broad and diverse range of activities. For example, it has been found that myeloid cells can be engineered to express a chimeric fusion protein (CFP) containing an antigen binding domain to have a broad and diverse range of activities. For example, it has been found that myeloid cells can be engineered to have enhanced phagocytic activity such that upon binding of the CFP to an antigen on a target cell, the cell exhibits increased phagocytosis of the target cell. It has also been found that myeloid cells can be engineered to promote T cell activation such that upon binding of the CFP to an antigen on a target cell, the cell promotes activation of T cells, such as T cells in the tumor microenvironment. The engineered myeloid cells can be engineered to promote secretion of tumoricidal molecules such that upon binding of the CFP to an antigen on a target cell, the cell promotes secretion of tumoricidal molecules from nearby cells. The engineered myeloid cells can be engineered to promote recruitment and trafficking of immune cells and molecules such that upon binding of the CFP to an antigen on a target cell, the cell promotes recruitment and trafficking of immune cells and molecules to the target cell or a tumor microenvironment.

The present disclosure is based on the important finding that myeloid cells can be engineered to express a gene of interest and can be targeted to site of inflammation, infection or tumor. Myeloid cells thus engineered can overcome at least some of the limitations of CAR-T cells, including being readily recruited to solid tumors; having an engineerable duration of survival, therefore lowering the risk of prolonged persistence resulting in aplasia and immunodeficiency; myeloid cells cannot be contaminated with T cells; myeloid cells can avoid fratricide, for example because they do not express the same antigens as malignant T cells; and myeloid cells have a plethora of anti-tumor functions that can be deployed. In some respects, engineered myeloid derived cells can be safer immunotherapy tools to target and destroy diseased cells. However, engineering myeloid cells for enhancing gene expression for non-viral DNA therapy remains a significant challenge.

Moreover, myeloid cells, such as macrophages, have been ubiquitously found in the tumor environment (TME) and are notably the most abundant cells in some tumor types. As part of their role in the immune system, myeloid cells, such as macrophages, are naturally engaged in clearing diseased cells. The present invention relates to harnessing myeloid cell function specifically for targeting, killing and directly and/or indirectly clearing diseased cells as well as the delivery payloads such as antigens and cytokines.

Engineered myeloid cells can also be short-lived in vivo, phenotypically diverse, sensitive, plastic, and are often found to be difficult to manipulate in vitro. For example, exogenous gene expression in monocytes has been difficult compared to exogenous gene expression in non-hematopoietic cells. There are significant technical difficulties associated with transfecting myeloid cells (e.g., monocytes/macrophages). As professional phagocytes, myeloid cells, such as monocytes/macrophages, comprise many potent degradative enzymes that can disrupt nucleic acid integrity and make gene transfer into these cells an inefficient process. This is especially true of activated macrophages which undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery.

The present disclosure provides innovative methods and compositions that can successfully electroporate, transfect or transduce a myeloid cell, or otherwise induce a genetic modification in a myeloid cell, with the purpose of augmenting a functional aspect of a myeloid cell, additionally, without compromising the cell's differentiation capability, maturation potential, and/or its plasticity. Provided herein is a composition comprising a nucleic acid comprising (i) DNA sequence encoding an mRNA or (ii) the mRNA sequence, wherein the mRNA sequence comprises (i) a 5′ UTR sequence and (ii) a 3′ UTR sequence, wherein the 5′ UTR is at least 45 nucleotides in length and a sequence encoding a target gene or protein therebetween. In some embodiments, the 5′ UTR sequence, and/or the 3′ UTR sequence can comprise a non-native sequence, that is, a sequence that is not present in an unmodified transcript. In some embodiments, the nucleic acid or nucleic acid sequence is recombinant. In some embodiments, the nucleic acid” or nucleic acid sequence is engineered. In some embodiments, the nucleic acid or nucleic acid sequence is synthetic. In some embodiments, the nucleic acid or nucleic acid sequence is in vitro transcribed. In some embodiments, the nucleic acid or nucleic acid sequence is isolated or purified.

In some embodiments, the nucleic acid, e.g., an engineered nucleic acid, an in vitro transcribed (IVT) mRNA, a synthetic or modified nucleic acid as described herein is not conjugated to or associated with a lipid nanoparticle (LNP).

In some embodiments, the nucleic acid, e.g., an engineered nucleic acid, an in vitro transcribed mRNA, a synthetic or modified nucleic acid as described herein is electroporated into a cell. In some embodiments, the nucleic acid, e.g. an IVT mRNA comprises a 3′UTR and a 5′UTR. In some embodiments, the 3′ UTR sequence is followed by a poly A sequence. In some embodiments, the poly A sequence is at least 100 nucleotides long. In some embodiments, the poly A sequence is at least about 50, 60, 70, 80, 90, 100, 110, 120, 130, 140, 150, 160, 170, 180, 190 or 200 nucleotides long. In some embodiments, the poly A sequence is greater than 200 nucleotides long. In some embodiments, within the 5′ UTR, a translation start site is at least 15 nucleotides downstream of the 5′ end of the mRNA. In some embodiments, a translation start site is at least 20 nucleotides downstream of the 5′ end of the mRNA. In some embodiments, the translation start site is at least 25 nucleotides downstream of the ribosome binding site. In some embodiments, the translation start site is at least 30 nucleotides downstream of the ribosome binding site. In some embodiments, the 5′ end of the nucleic acid comprises a methyl guanylate cap. In some embodiments, the nucleic acid comprises a single translational start site. In some embodiments, the mRNA coding sequence is 100-10,000 nucleotides long. In some embodiments, the nucleic acid sequence comprises a 5′ UTR sequence selected from SEQ ID NOs 46-51. In some embodiments, the nucleic acid sequence comprises a 3′ UTR sequence selected from SEQ ID NOs 52-59. Provided herein is a composition comprising a cell comprising the composition described in the above section, wherein in some embodiments the cell is a myeloid cell, a CD14+ cell, a CD16− cell, or a CD14+/CD16− cell or the cell is a T cell. Also provided herein is a pharmaceutical composition comprising the composition described above; and a pharmaceutical acceptable excipient.

Provided herein is a method of treating a subject with a disease or condition comprising administering the pharmaceutical composition described herein to a subject in need thereof. Also provided herein is a method of expressing a protein encoded by a nucleic acid in a cell, the method comprising (a) incorporating into the cell ex vivo a nucleic acid comprising (i) DNA encoding an mRNA or (ii) the mRNA, and (b) expressing a protein encoded by a sequence of the mRNA; wherein the mRNA comprises (i) a 5′ UTR sequence; a sequence encoding a protein or polypeptide and (ii) a 3′ UTR sequence, wherein expression of the protein or polypeptide is detectable upto at least 24 hours, at least 48 hours, or at least 72 hours after (a).

In some embodiments expression of the protein is detectable according to an immunoassay after at least 72 hours after (a). In some embodiments, expression of the protein is detectable in at least 10% to at least 50% of the cells according to an immunoassay after at least 72 hours after (a). In some embodiments, expression of the protein is detectable in at least 40% of the cells according to an immunoassay after at least 72 hours after (a). In some embodiments, expression of the protein is detectable in at least 30% of the cells according to an immunoassay after at least 72 hours after (a). In some embodiments, expression of the protein is detectable in at least 20% of the cells after at least 72 hours after (a). In some embodiments expression of the protein is detectable according to an immunoassay after at least 48 hours after (a). In some embodiments, expression of the protein is detectable in at least 10% to at least 90% of the cells according to an immunoassay after at least 48 hours after (a). In some embodiments, expression of the protein is detectable in at least 80% of the cells, at least 70% of the cells, at least 60% of the cells, at least 50% of the cells, at least 40% of the cells, at least 30% of the cells, or at least 20% of the cells according to an immunoassay after at least 48 hours after (a).

In some embodiments, expression of a protein encoded by the nucleic acid is detectable, such as by an immunoassay, up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after incorporation of the nucleic acid into the cell. In some embodiments, expression of a protein encoded by the nucleic acid is detectable, such as by an immunoassay, up to and/or at least 96 hours after incorporation of the nucleic acid into the cell. In some embodiments, expression of a protein encoded by the nucleic acid is detectable, such as by an immunoassay, up to and/or at least 3 days after incorporation of the nucleic acid into the cell. In some embodiments, expression of a protein encoded by the nucleic acid is detectable, such as by an immunoassay, up to and/or at least 4 days after incorporation of the nucleic acid into the cell. In some embodiments, expression of a protein encoded by the nucleic acid is detectable, such as by an immunoassay, up to and/or at least 5 days after incorporation of the nucleic acid into the cell. In some embodiments, expression of a protein encoded by the nucleic acid is detectable, such as by an immunoassay, up to and/or at least 6 days after incorporation of the nucleic acid into the cell. In some embodiments, expression of a protein encoded by the nucleic acid is detectable, such as by an immunoassay, up to and/or at least 7 days after incorporation of the nucleic acid into the cell. In some embodiments, expression of a protein encoded by the nucleic acid is detectable, such as by an immunoassay, up to and/or at least 8 days after incorporation of the nucleic acid into the cell. In some embodiments, expression of a protein encoded by the nucleic acid is detectable, such as by an immunoassay, up to and/or at least 9 days after incorporation of the nucleic acid into the cell. In some embodiments, expression of a protein encoded by the nucleic acid is detectable, such as by an immunoassay, up to and/or at least 10 days or more after incorporation of the nucleic acid into the cell. In some embodiments, expression of a protein encoded by the nucleic acid is detectable, such as by an immunoassay, in at least 10% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 20% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 30% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 40% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 50% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 60% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 70% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 80% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells. In some embodiments, expression of a protein encoded by the nucleic acid is detectable, such as by an immunoassay, in at least 90% of the cells of a population of cells up to and/or at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours, or up to and/or at least 3, 4, 5, 6, 7, 8, 9, or 10 or more days after incorporation of the nucleic acid into the population of cells.

Provided herein is a method of expressing a protein encoded by a nucleic acid in a cell, the method comprising (a) incorporating into the cell ex vivo a nucleic acid comprising (i) DNA encoding an mRNA or (ii) the mRNA, and (b) expressing a protein encoded by a sequence of the mRNA; wherein the mRNA comprises (i) a 5′ UTR sequence and (ii) a 3′ UTR sequence, wherein the 5′ UTR is at least 45 nucleotides in length. In some embodiments, the nucleic acid comprises a 5′ UTR sequence selected from SEQ ID NOs 46-51.

In some embodiments, the nucleic acid sequence comprises a 3′ UTR sequence selected from SEQ ID NOs 52-59. In some embodiments, the 3′ UTR is followed by a poly A tail and the poly A tail is enzymatically added to the 3′end of the mRNA. In some embodiments, the 3′ UTR is followed by a poly A tail and the poly A tail is an encoded poly A tail.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1A is an exemplary diagram showing 5′- and 3′-UTR regions in an mRNA designed for increased stability of the mRNA and higher expression by promoting interaction with mRNA binding proteins.

FIG. 1B depicts a mechanism for in vitro transcribed RNA in which the 5′- and 3′-UTRs are added by PCR extension.

FIG. 2 shows an experimental design to test the expression of the engineered constructs in myeloid cells.

FIG. 3 shows expression data of the different constructs encoding a CD5 binder CAR by flow cytometry.

FIG. 4 shows expression data of the different constructs encoding a CD5 binder CAR by flow cytometry.

FIG. 5 shows enhanced expression of a binder construct using enzymatically added poly A tail to the mRNA encoding the binder construct.

FIG. 6 shows prolonged expression of the binder construct.

FIG. 7 shows expression of a binder encoded by an mRNA with an enzymatic poly A tail versus an mRNA with plasmid-encoded poly A tail in primary monocytes over time. Primary monocytes were mock electroporated or electroporated with a CD5 binder expressing mRNA with an enzymatic poly A tail or a CD5 binder expressing mRNA with a plasmid-encoded poly A tail and expression of the CD5 binder was analyzed by flow cytometry.

FIG. 8 shows expression of a binder encoded by an mRNA with an enzymatic poly A tail containing the indicated modified nucleobases at the indicated location(s) in THP-1 cells over time. The poly A tail was added enzymatically using the indicated poly A polymerases. THP-1 cells were mock electroporated or electroporated with a CD5 binder expressing mRNA with an enzymatic poly A tail containing the indicated modified nucleobases at the indicated location(s) and expression of the CD5 binder was analyzed by flow cytometry.

FIG. 9 is a schematic showing different CAP structures used for modifications of the 5′end of the mRNA encoding a sequence of interest for testing the effects on expression of the mRNA in myeloid cells. The Cap1 structure shown in the left image was introduced at the 5′-terminus of the mRNA enzymatically using Vaccinia capping enzyme and 2′-O-methyltransferase. The Cap 0 shown in the right image was introduced co-transcriptionally using anti-reverse cap analog (ARCA).

FIG. 10 shows expression of a CD5-binding CFP (CD5 binder) encoded by an mRNA with the indicated CAP structures in THP-1 cells over time. THP-1 cells were mock electroporated or electroporated with a CD5 binder expressing mRNA having either the Cap1 or the Cap 0 structures and expression of the CD5 binder was analyzed by flow cytometry.

FIG. 11 shows expression of a CD5-binding CFP (CD5 binder) encoded by an mRNA with the indicated CAP structures in primary monocytes over time. Primary monocytes were mock electroporated or electroporated with a CD5 binder expressing mRNA having either the Cap1 or the Cap 0 structures and expression of the CD5 binder was analyzed by flow cytometry.

FIG. 12 shows expression of a CD5-binding CFP (CD5 binder) encoded by an mRNA that was in vitro transcribed in the presence of unmodified uridine, pseudouridine, 1-methyl-pseudouridine or 5-methoxyuridine in THP-1 cells over time. THP-1 cells were mock electroporated or electroporated with a CD5 binder expressing mRNA that was in vitro transcribed in the presence of unmodified uridine, pseudouridine, 1-methyl-pseudouridine or 5-methoxyuridine and expression of the CD5 binder was analyzed by flow cytometry.

FIG. 13A illustrates the structure of uridine base (top) and base modified uridine analogs as indicated.

FIG. 13B shows data demonstrating that replacement of all Uridine residues in the mRNA did not improve CD5 binder expression in monocytes. Percent of the binder positive monocytes are shown.

FIG. 13C shows data demonstrating expression of an mRNA encoded HER2-specific CFP (HER2 binder) where the mRNA was produced with various proportions of pseudouridine in the IVT reaction as indicated at the top in %. Data shown as percent of the binder positive monocytes.

FIG. 13D shows a graph of percent of the HER2-specific binder positive monocytes from the flow cytometry data shown in FIG. 13C.

FIG. 13E shows expression of mRNA produced with various % of pseudouridine in the IVT reaction, mean fluorescence intensity (MFI) of the cells expressing the mRNA encoded protein is shown.

FIG. 13F shows graph of the mean fluorescence intensity (MFI) of the mRNA encoded protein expression in monocytes over time, summarized from data shown in FIG. 13E.

FIG. 13G shows data demonstrating expression of an mRNA encoded HER2-specific CFP (HER2 binder) where the mRNA was produced with various proportions of 1-methyl-pseudouridine in the IVT reaction as indicated at the top in %. Data shown as percent of the binder positive monocytes.

FIG. 13H shows graph of percent of the HER2-specific binder positive monocytes summarized from the flow cytometry data shown in FIG. 13G.

FIG. 13I shows mean fluorescence intensity (MFI) of the mRNA encoded protein expression in binder positive monocytes.

FIG. 13J shows graph of the mean fluorescence intensity (MFI) of the mRNA encoded protein expression in monocytes over time, summarized from data shown in FIG. 13I.

FIG. 13K shows data demonstrating expression of an mRNA encoded HER2-specific CFP (HER2 binder) where the mRNA was produced with various proportions of 15-methoxyudouridine in the IVT reaction as indicated at the top in %. Data shown as percent of the binder positive monocytes.

FIG. 13L shows graph of percent of the HER2-specific binder positive monocytes summarized from the flow cytometry data shown in FIG. 13K.

FIG. 13M shows mean fluorescence intensity (MFI) of the mRNA encoded protein expression in binder positive monocytes.

FIG. 13N shows graph of the mean fluorescence intensity (MFI) of the mRNA encoded protein expression in monocytes over time, summarized from data shown in FIG. 13M.

FIG. 13O shows data indicating mRNA produced with 20% of pseudouridine in the IVT reaction displays the best binder expression. Left, % of binder positive cells; right, MFI.

FIG. 14A shows expression of a CD5-binding CFP (CD5 binder) encoded by an mRNA that was in vitro transcribed in the presence of unmodified uridine, pseudouridine, 1-methyl-pseudouridine or 5-methoxyuridine in primary monocytes over time. Primary monocytes were mock electroporated or electroporated with a CD5 binder expressing mRNA that was in vitro transcribed in the presence of unmodified uridine, pseudouridine, 1-methyl-pseudouridine or 5-methoxyuridine and expression of the CD5 binder was analyzed by flow cytometry.

FIG. 14B depicts a graph showing expression of a CD5-binding CFP (CD5 binder) encoded by an mRNA that was in vitro transcribed in the presence of unmodified uridine or an mRNA that was in vitro transcribed in the presence of 5-methoxyuridine in primary monocytes over time. Primary monocytes were electroporated with bulk or purified mRNA encoding a CD5 binder that was in vitro transcribed in the presence of unmodified uridine or an mRNA encoding the CD5 binder that was in vitro transcribed in the presence of 5-methoxyuridine and expression of the CD5 binder was analyzed by flow cytometry.

FIG. 14C shows expression of a CD5-binding CFP (CD5 binder) encoded by an mRNA in primary monocytes over time, using an experimental setup outlined in FIG. 2. Primary monocytes were mock electroporated or electroporated with a CD5 binder expressing mRNA and expression of the CD5 binder was analyzed by flow cytometry.

FIG. 15A shows expression of a CD5-binding CFP (CD5 binder) encoded by an mRNA with a plasmid-encoded poly A tail containing the indicated 5′ and 3′-UTRs in primary human monocytes over time. Primary human monocytes from Donor 1 were electroporated with a CD5 binder expressing mRNA containing standard 5′ and 3′ UTRs or a C3 5′ UTR and an ORM1 3′ UTR and expression of the CD5 binder was analyzed by flow cytometry. The results indicate that the C3 5′ UTR and ORM1 3′ UTR improve extent and duration of CD5 binder expression in monocytes from Donor 1.

FIG. 15B shows expression of a CD5-binding CFP (CD5 binder) encoded by an mRNA with a plasmid-encoded poly A tail containing the indicated 5′ and 3′-UTRs in primary human monocytes over time. Primary human monocytes from Donor 2 were electroporated with a CD5 binder expressing mRNA containing standard 5′ and 3′ UTRs or a C3 5′ UTR and an ORM1 3′ UTR and expression of the CD5 binder was analyzed by flow cytometry. The results indicate that the C3 5′ UTR and ORM1 3′ UTR improve extent and duration of CD5 binder expression in monocytes from Donor 2.

FIG. 16A shows expression of a HER2-binding CFP (HER2 binder) encoded by an mRNA with a enzymatically added poly A tail containing the indicated 5′ and 3′-UTRs in THP-1 cells over time. THP-1 cells were electroporated with 100 microgram/mL of a HER2 binder expressing mRNA containing standard 5′ and 3′ UTRs or a C3 5′ UTR and an ORM1 3′ UTR and expression of the HER2 binder was analyzed by flow cytometry. The results indicate that the C3 5′ UTR and ORM1 3′ UTR improve extent and duration of HER2 binder expression in THP 1 cells electroporated with 100 microgram/mL of the mRNA.

FIG. 16B shows expression of a HER2-binding CFP (HER2 binder) encoded by an mRNA with a enzymatically added poly A tail containing the indicated 5′ and 3′-UTRs in THP-1 cells over time. THP-1 cells were electroporated with 50 microgram/mL of a HER2 binder expressing mRNA containing standard 5′ and 3′ UTRs or a C3 5′ UTR and an ORM1 3′ UTR and expression of the HER2 binder was analyzed by flow cytometry. The results indicate that the C3 5′ UTR and ORM1 3′ UTR improve extent and duration of HER2 binder expression in THP 1 cells electroporated with 50 microgram/mL of the mRNA.

FIG. 16C shows expression of a HER2-binding CFP (HER2 binder) encoded by an mRNA with a enzymatically added poly A tail containing the indicated 5′ and 3′-UTRs in THP-1 cells over time. THP-1 cells were electroporated with 25 microgram/mL of a HER2 binder expressing mRNA containing standard 5′ and 3′ UTRs or a C3 5′ UTR and an ORM1 3′ UTR and expression of the HER2 binder was analyzed by flow cytometry. The results indicate that the C3 5′ UTR and ORM1 3′ UTR improve extent and duration of HER2 binder expression in THP 1 cells electroporated with 25 microgram/mL of the mRNA.

FIG. 17 shows expression of a HER2-binding CFP (HER2 binder) encoded by an mRNA with a enzymatically added poly A tail containing the indicated 5′ and 3′-UTRs in primary monocytes from Donor 3 over time. Primary human monocytes from Donor 3 were electroporated with a HER2 binder expressing mRNA containing standard 5′ and 3′ UTRs or a C3 5′ UTR and an ORM1 3′ UTR and expression of the HER2 binder was analyzed by flow cytometry. The results indicate that the C3 5′ UTR and ORM1 3′ UTR improve extent and duration of HER2 binder expression in primary monocytes from Donor 3.

FIG. 18 shows expression of a HER2-binding CFP (HER2 binder) encoded by an mRNA with a enzymatically added poly A tail containing the indicated 5′ and 3′-UTRs in primary monocytes from Donor 3 over time. Primary human monocytes from Donor 4 were electroporated with a HER2 binder expressing mRNA containing standard 5′ and 3′ UTRs or a C3 5′ UTR and an ORM1 3′ UTR and expression of the HER2 binder was analyzed by flow cytometry. The results indicate that the C3 5′ UTR and ORM1 3′ UTR improve extent and duration of HER2 binder expression in primary monocytes from Donor 4.

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Myeloid cells are recently shown to provide a strong platform for therapeutic intervention in various diseases including cancer, because these cells are able to migrate chemotactically to a locus of infection, inflammation, and tumor, can phagocytose and kill bacteria and parasites, can scavenge dead or apoptotic cells and can attack tumor cells or cells harboring mutation or an aberrant phenotype. A myeloid cell is usually short lived inside the body. A myeloid cell can be engineered ex vivo to augment one or more of the functions related to phagocytosis or chemotaxis and introduced into a subject having a disease, for the engineered myeloid cell to target and attack the diseased cells in the subject, such as tumor cells or infected cells. However, myeloid cells are complex and can rapidly undergo change of state in which its effectiveness as a phagocytic cell may be diminished. In this respect, engineering or manipulating a myeloid cell can pose various challenges. For example, introducing plasmid DNA in a myeloid cell can lead to transformation of a monocyte cell into a M2 phenotype, where the cells may lose chemotactic movement, and surface expression of chemoattracted receptors. A monocyte or macrophage with altered phenotype following introduction of DNA comprising a gene of interest may become less effective in its innate functions such as phagocytosis and chemotaxis.

Provided herein are methods and compositions of engineering a myeloid cell. In some embodiments, the engineering is ex vivo. In some embodiments, an exogenous polynucleotide sequence may be introduced in vitro to generate an engineered myeloid cell. In some embodiments, an exogenous polynucleotide sequence may be introduced in vivo that is designed to be specifically taken up by a myeloid cell in vivo, and the myeloid cell expresses the gene of interest encoded by the polynucleotide. Of importance, compositions and methods are provided herein to express a polynucleotide sequence in a myeloid cell. The myeloid cell expressing the exogenous polynucleotide sequence should retain its chemotactic, phagocytic and cytotoxic function in order to perform a therapeutically effective function in a living body.

Provided herein are polynucleotide compositions for improved expression of an encoded protein or polypeptide in a myeloid cell. In some embodiments, the polynucleotide is messenger RNA.

The instant disclosure is directed to a non-viral method and compositions for expressing a exogenous genetic material, such as a polynucleotide in a myeloid cell to ensure robust and prolonged expression in a myeloid cell, and to ensure that the myeloid cell expressing the exogenous genetic material is fully functional and capable of actively migrating to the cite of infection or inflammation or tumor, that it responds to chemotactic signals in vivo for it to be localized, for example to a tumor, and that it is actively phagocytic.

Prolonged expression of a polynucleic acid in a myeloid cell has been found to be highly challenging. First there are technical difficulty associated with transfecting monocytes and macrophages. As professional phagocytes, monocytes and macrophages are endowed with many potent degradative enzymes that can disrupt nucleic acid integrity and make gene transfer into these cells an inefficient process. Monocytes and macrophages undergo a dramatic change in their physiology following exposure to immune or inflammatory stimuli. Viral transduction of these cells has been hampered because macrophages are end-stage cells that generally do not divide; therefore, some of the vectors that depend on integration into a replicative genome have met with limited success. Furthermore, macrophages are quite responsive to “danger signals,” and therefore several of the original viral vectors that were used for gene transfer induced potent anti-viral responses in these cells making these vectors inappropriate for gene delivery. For multiple reasons as is known to one of skill in the art, alternatives to viral gene delivery is always sought for safety issues. However, most of the original transfection techniques enjoyed only limited success with macrophages. This is due to the requirement of fairly high gene copy numbers required to efficiently transfect cells and the relatively high degree of toxicity associated with the process whereby the host cell membrane is made permeable to DNA. The various transfection methods used to introduce foreign DNA into mammalian cells include: DEAE-dextran, calcium phosphate coprecipitation, cationic lipid vehicles, and physical disruption of the host cell membranes by electroporation or nucleofection. All these approaches have been used with varying degrees of success on macrophages.

Provided herein are methods and compositions for producing engineered myeloid cells (including, but not limited to, neutrophils, monocytes, myeloid dendritic cells (mDCs), mast cells and macrophages), designed to specifically express a gene of interest (GOI), such as a protective gene in a disease. A gene of interest may be a gene or a polynucleic acid bearing a sequence encoding corrected wild-type protein sequence that can substitute a mutated non-functional sequence in a subject that does not express a functional protein. A gene of interest may be a immunoprotective gene, a chemokine, a cytokine, a secreted protein, a cytoplasmic protein, a membrane protein or a nuclear protein.

In one embodiment, a myeloid cell can be engineered to express a CAR (chimeric antigen receptor), also termed chimeric fusion protein (CFP) that can bind a target protein or biomolecule or a target cell. These engineered myeloid cells can therefore attack and kill target cells directly (e.g., by phagocytosis) and/or indirectly (e.g., by activating T cells). In some embodiments, the target cell is a cancer cell.

While a polynucleotide encoding a CFP may be used throughout the specification as an exemplary embodiment, the composition and methods disclosed herein are applicable for expressing a polynucleotide encoding any gene of interest, e.g., a synthetic polynucleotide.

While cancer is one exemplary embodiment described in detail in the instant disclosure, the methods and technologies described herein are contemplated to be useful in targeting an infected or otherwise diseased cell inside the body. In some embodiments, the methods and compositions may be utilized in introducing any polynucleotide sequence encoding a protein to enhance macrophage function, such as a transcription enhancer, or a cytokine or a chemokine. The method can be used to target the myeloid cell to a pathogen, and engulf the pathogen and at the same time present suitable antigens to T cells for inducing the lymphocyte cascade and protective immunity. In some embodiments, an exemplary pathogen may be Bacillus anthracis, Clostridium botulinum, Fracisella tularensis, Variola major, Salmonella sp., Mycobacterium tuberculosis, Listeria monocytogenes, Campylobacter jejuni, Yersinia enterocolitica, or protozoans, for example, Cryptosporidium parvum, Cyclospora cayatanensis, Giardia lamblia, Entamoeba histolytica, Toxoplasma gondi, Naegleria fowleri, Balamuthia mandrillaris, or fungi. In some embodiments, the methods and compositions disclosed herein may be useful in generating engineered myeloid cells for expressing a gene to compensate for a deficient gene or replace a mutated gene product with a correct wild-type protein, such as in monogenic disorders.

Provided herein are compositions and methods for treating diseases or conditions, such as cancer. The compositions and methods provided herein utilize human myeloid cells, including, but not limited to, neutrophils, monocytes, myeloid dendritic cells (mDCs), mast cells and macrophages, to target diseased cells, such as cancer cells. The compositions and methods provided herein can be used to eliminate diseased cells, such as cancer cells and or diseased tissue, by a variety of mechanisms, including T cell activation and recruitment, effector immune cell activation (e.g., CD8 T cell and NK cell activation), antigen cross presentation, enhanced inflammatory responses, reduction of regulatory T cells and phagocytosis. For example, the myeloid cells can be used to sustain immunological responses against cancer cells.

In one aspect, provided herein is a method of expressing a polynucleic acid in a myeloid cell, such as a CD14+ human myeloid cell. In some embodiments, provided herein is an engineered polynucleic acid. In some embodiments, the engineered polynucleic acid is suitable for efficient expression on a polypeptide encoded by the engineered polynucleic acid in a myeloid cell. In some embodiments, the polynucleic acid is RNA. In some embodiments, the RNA is mRNA. In some embodiments, the mRNA encodes a protein that is expressed in the myeloid cell. In some embodiments, the mRNA encoded protein is a cytoplasmic protein. In some embodiments, the mRNA encoded protein is a nuclear protein. In some embodiments, the mRNA encoded protein is a membrane protein. In some embodiments, the mRNA encoded protein is a secreted protein.

In some embodiments an mRNA comprising a sequence encoding a gene of interest is expressed in a myeloid cell, wherein the mRNA is modified for efficient expression in a myeloid cell. In some embodiments, the mRNA is modified at the termini for enhanced and/or prolonged expression in a myeloid cell. In some embodiments, the termini include a 5′terminus, and/or a 3′terminus modification.

In some embodiments, the modification may be in the mRNA chemistry, that is in the nucleotides of the mRNA. In some embodiments, the modification is introduced into the mRNA at the time of mRNA formation. In some embodiments, the modification is introduced into the mRNA after the mRNA is formed.

In one embodiment, the poly A tail of the mRNA is designed for improving the efficiency of expressing a polypeptide encoded by a sequence within the mRNA in a myeloid cell.

In some embodiments, the 5′-UTR is specifically designed or chosen for improving the efficiency of expressing a polypeptide encoded by a sequence within the mRNA in a myeloid cell.

In some embodiments, the 3′-UTR is specifically designed or chosen for improving the efficiency of expressing a polypeptide encoded by a sequence within the mRNA in a myeloid cell.

Also provided herein is a pharmaceutical composition comprising a composition described herein, such as an engineered nucleic acid described herein, a vector described herein, a polypeptide described herein or a cell described herein; and a pharmaceutically acceptable excipient. The term “nucleic acid” may be used to designate a synthesized, recombinant, engineered or in vitro transcribed nucleic acid. In some embodiments, a nucleic acid is not naturally occurring.

Provided herein is a composition comprising a nucleic acid comprising: (i) DNA encoding an mRNA or (ii) the mRNA, wherein the mRNA comprises (i) a 5′ UTR sequence and (ii) a 3′ UTR sequence, wherein the 5′ UTR is at least 45 nucleotides in length and a sequence encoding a protein or polypeptide, wherein the protein or polypeptide encoded by the nucleic acid when expressed in the myeloid cell is detected in the cell for at least up to 72 hours. Provided herein is a composition comprising a nucleic acid comprising: an mRNA wherein the mRNA comprises (i) a 5′ UTR sequence and (ii) a 3′ UTR sequence, where (i) and (ii) flanks (iii) a sequence encoding a protein or polypeptide; wherein the 5′ UTR sequence is selected from SEQ ID NOs 46-51; and the 3′ UTR sequence selected from SEQ ID NOs 52-59; wherein the protein or polypeptide encoded by the nucleic acid when expressed in the myeloid cell is detected in the cell for at least up to 72 hours. In some embodiments, the nucleic acid comprises a 5′ UTR sequence of SEQ ID NO: 47 and a 3′ UTR sequence of SEQ ID NO: 53. Provided herein is a composition comprising a nucleic acid comprising: (i) DNA encoding an mRNA or (ii) the mRNA, wherein the mRNA comprises (i) a 5′ UTR sequence and (ii) a 3′ UTR sequence, wherein the 5′ UTR is at least 45 nucleotides in length and a sequence encoding a protein or polypeptide, and wherein the mRNA comprises an enzymatically added poly A tail; wherein the protein or polypeptide encoded by the nucleic acid when expressed in the myeloid cell is detected in the cell for at least up to 72 hours. In some embodiments, the composition is not conjugated to or associated with a lipid nanoparticle (LNP). In some embodiments, the nucleic acid comprises one or more modified nucleotide bases, wherein a fraction of the total number of uridine bases are modified to a pseudouridine, a 1-methyl-pseudouridine or a 5-methoxyuridine. In some embodiments, less than 50% of the total number of uridine bases are modified to a pseudouridine, a 1-methyl-pseudouridine or a 5-methoxyuridine. In some embodiments, the protein or polypeptide encoded by the nucleic acid when expressed in the myeloid cell is detected in the cell for at least up to 72 hours. In some embodiments the 5′ UTR is at least 20 nucleotides in length. In some embodiments the 5′ UTR is at least 30 nucleotides in length. In some embodiments the 5′ UTR is at least 60 nucleotides in length. In some embodiments the 5′ UTR is at least 100 nucleotides in length.

In one embodiment, the nucleic acid is an mRNA comprising a poly A sequence that is enzymatically added. In one embodiment, the nucleic acid is an mRNA comprising a poly A sequence that is enzymatically added. In some embodiments, the nucleic acid is an mRNA comprising a poly A sequence that is encoded by the plasmid which comprises the template for generating the mRNA by in vitro transcription (IVT). The template for IVT is a linearized plasmid. Typically, the length of the poly A is controlled when encoded by the plasmid, and is less controlled when enzymatically added. An mRNA product comprising an enzymatically added poly A tail may be tailored at best to contain a narrowed range of the number of A-residues. The in vitro transcribed mRNA is thereafter purified prior.

In some embodiments, the nucleic acid is encapsulated in a lipid nanoparticle. In some embodiments, the lipid nanoparticle comprises a cationic lipid, and at least one of: a non-cationic lipid, a neutral lipid, cholesterol and a conjugated lipid. In some embodiments, the LNP is less than 250 nm is diameter. In some embodiments, the LNP is less than 250 nm is diameter. In some embodiments, the LNP is less than 150 nm is diameter. In some embodiments, the LNP is about 100 nm is diameter. In some embodiments, the nucleic acid is encapsulated in a liposome.

In some embodiments, the nucleic acid comprises a poly A sequence downstream of the 3′ UTR sequence. In some embodiments, the poly A sequence is at least 50 nucleotides long. In some embodiments, the poly A sequence is at least 60, 70, 80, or 90 nucleotides long. In some embodiments, the poly A sequence is at least 100 nucleotides long. In some embodiments, the poly A sequence is at least 110 nucleotides long. In some embodiments, the poly A sequence is at least 120 nucleotides long. In some embodiments, the poly A sequence is at least 130 nucleotides long. In some embodiments, the poly A sequence is at least 140 nucleotides long. In some embodiments, the poly A sequence is at least 150, 160, 170, 180, 190 or 200 nucleotides long. In some embodiments, the within the 5′ UTR, a translation start site is at least 15 nucleotides downstream of the 5′ end. In some embodiments, the translation start site is at least 20 nucleotides downstream of the 5′ end. In some embodiments, the translation start site is at least 25 nucleotides downstream of the 5′ end. In some embodiments, the translation start site is at least 30 nucleotides downstream of the 5′ end. In some embodiments, the nucleic acid comprises a single translational start site.

In some embodiments, the nucleic acid comprises a 5′ methyl guanylate cap. In some embodiments, the nucleic acid comprises a Cap 0 structure. In some embodiments, the Cap 0 structure is introduced into the mRNA cotranscriptionally using anti-reverse capping analog (ARCA).

In some embodiments, the nucleic acid is 20-20,000 nucleotides long. In some embodiments, the nucleic acid is 20-22 nucleotides long. In some embodiments, the nucleic acid is 20-50 nucleotides long. In some embodiments, the nucleic acid is 20-100 nucleotides long. In some embodiments the nucleic acid is a small interfering RNA. In some embodiments, the nucleic acid is an inhibitory RNA. In some embodiments, the nucleic acid is a tRNA. In some embodiments, the nucleic acid is single stranded. In some embodiments, the nucleic acid is double stranded.

In some embodiments, the nucleic acid is an mRNA. In some embodiments, the mRNA is 50-20,000 nucleotides long.

In some embodiments, the nucleic acid comprises a single translational start site. In some embodiments, the nucleic acid comprises a 5′ UTR sequence selected from SEQ ID NOs 46-51. In some embodiments, the nucleic acid comprises a 3′ UTR sequence selected from SEQ ID NOs 52-59. In some embodiments, the nucleic acid comprises a 5′ UTR sequence of SEQ ID NO: 47 and a 3′ UTR sequence of SEQ ID NO: 53.

In some embodiments, the nucleic acid is an engineered nucleic acid. In some embodiments, the nucleic acid is recombinant nucleic acid. In some embodiments, the nucleic acid is in vitro transcribed mRNA. In some embodiments, the nucleic acid is a synthesized nucleic acid.

In some embodiments, the nucleic acid is isolated.

In some embodiments, the nucleic acid is purified.

In some embodiments, the nucleic acid comprises at least 1 modified nucleotide.

In some embodiments, the nucleic acid comprises at least 10% modified nucleotides.

In some embodiments, the nucleic acid comprises at least 20% modified nucleotides.

In some embodiments, the nucleic acid comprises at least 30%, 40%, or 50% modified nucleotides. In some embodiments, less than 70% of the uridine residues in the nucleic acid are modified.

In some embodiments, less than 50% of the uridine residues in the nucleic acid are modified.

In some embodiments, the modified nucleotide is a pseudouridine, 1-methyl-pseudouridine or a 5-methoxyuridine that replaces a uridine.

In some embodiments, the nucleic acid comprises about 10%, about 12%, about 14%, about 16%, about 18%, about 20%, about 21%, about 22%, about 23%, about 24%, about 25%, about 26% about 27%, about 28%, about 29%, about 30%, about 31%, about 32%, about 33%, about 34%, about 35%, about 36%, about 37%, about 38%, about 39% about 40% or more of the uridine residues of the nucleic acid that are modified to a pseudouridine, 1-methyl-pseudouridine or a 5-methoxyuridine.

Provided herein is a composition comprising a cell comprising the composition of any one of embodiments described above.

In some embodiments, the cell is a myeloid cell, a CD14+ cell, a CD16− cell, or a CD14+/CD16− cell.

In some embodiments, the cell is a T cell.

In some embodiments, the expression of the protein in the cell is detectable for at least 72 hours.

Provided herein is a pharmaceutical composition comprising the composition of any one of embodiments described above; and a pharmaceutical acceptable excipient.

Provided herein is a method of treating a subject with a disease or condition comprising administering the pharmaceutical composition described above to a subject in need thereof.

Provided herein is a method of expressing a protein encoded by a nucleic acid in a CD14+ cell, the method comprising: (a) incorporating into the CD14+ cell a nucleic acid comprising (i) DNA encoding an mRNA or (ii) the mRNA wherein the mRNA comprises a sequence encoding the protein, and (b) expressing the protein encoded by a sequence of the mRNA; wherein the mRNA comprises (i) a 5′ UTR sequence and (ii) a 3′ UTR sequence, wherein expression of the protein is detectable in the CD14+ cell up to at least 72 hours after (a).

In some embodiments, expression of the protein is detectable according to an immunoassay up to at least 72 hours after (a).

In some embodiments, expression of the protein is detectable in at least 20% of the cells after at least 72 hours after (a).

Provided herein is a method of expressing a protein encoded by a nucleic acid in a CD14+ cell, the method comprising incorporating into the CD14+ cell a nucleic acid comprising (i) DNA encoding an mRNA or (ii) the mRNA, and expressing a protein encoded by a sequence of the mRNA; wherein the mRNA comprises (i) a 5′ UTR sequence and (ii) a 3′ UTR sequence, flanking a sequence encoding the protein; wherein the 5′ UTR is at least 45 nucleotides in length.

In some embodiments, the a nucleic acid comprises a 5′ UTR sequence selected from SEQ ID NOs 46-51. In some embodiments, the nucleic acid sequence comprises a 3′ UTR sequence selected from SEQ ID NOs 52-59.

In some embodiments, the nucleic acid comprises a poly A tail, wherein the poly A tail is enzymatically added to the 3′end of the mRNA or is an encoded poly A tail.

In some embodiments, the nucleic acid sequence comprises a 5′ UTR sequence of SEQ ID NO: 47 and a 3′ UTR sequence of SEQ ID NO: 53. Provided herein is a method of expressing a protein encoded by a nucleic acid in a CD14+ cell, the method comprising incorporating into the CD14+ cell a nucleic acid comprising: incorporating into the CD14+ cell a nucleic acid comprising an mRNA encoding a protein, wherein the protein coding sequence is flanked by a 5′ UTR sequence of SEQ ID NO: 47 and a 3′ UTR sequence of SEQ ID NO: 53. Provided herein is a method for expressing a nucleic acid in a myeloid cell such that the nucleic acid is detectable in the cell at least up to 72 hours after introducing the nucleic acid in the myeloid cell. Provided herein is a method for expressing an mRNA encoding the protein in a myeloid cell such that the protein is detectable in the cell at least up to 72 hours after introducing the mRNA in the myeloid cell. In some embodiments, the 5′ UTR is at least 50, at least 60, at least 70, at least 80 or at least 100 nucleotides in length. In some embodiments, the nucleic acid comprises a poly A tail of 50-200 adenylate residues, and wherein the poly A tail is enzymatically added to the mRNA.

In some embodiments, the mRNA is targeted for myeloid cell-specific expression. In some embodiments, the mRNA encodes a sequence that is specifically expressed in a myeloid cell.

In some embodiments, the mRNA is introduced in the myeloid cell as a naked nucleic acid.

In some embodiments, the mRNA is introduced in the myeloid cell via a delivery vehicle, e.g., an LNP. In some embodiments, the lipid nanoparticle comprises a cationic lipid, and at least one of: a non-cationic lipid, a neutral lipid, cholesterol and a conjugated lipid.

In some embodiments, incorporating comprises transfecting. In some embodiments, incorporating comprises electroporating.

In some embodiments, incorporating comprises culturing the cell in the presence of the nucleic acid, wherein the nucleic acid is present at a concentration of at most about 500 micrograms/mL.

In some embodiments, incorporating comprises culturing the cell in the presence of the nucleic acid, wherein the nucleic acid is present at a concentration of at most about 250 micrograms/mL. In some embodiments, incorporating comprises culturing the cell in the presence of the nucleic acid, wherein the nucleic acid is present at a concentration of at most about 100 micrograms/mL. In some embodiments, incorporating comprises culturing the cell in the presence of the nucleic acid, wherein the nucleic acid is present at a concentration of at most about 50 micrograms/mL.

In some embodiments, incorporating comprises culturing the cell in the presence of the nucleic acid, wherein the nucleic acid is present at a concentration of from about 1 microgram/mL to about 500 micrograms/mL.

In some embodiments, incorporating comprises culturing the cell in the presence of the nucleic acid, wherein the nucleic acid is present at a concentration of from about 10 microgram/mL to about 100 micrograms/mL.

In some embodiments, incorporating comprises culturing the cell in the presence of the nucleic acid, wherein the nucleic acid is present at a concentration of from about 10 microgram/mL to about 50 micrograms/mL. In some embodiments, incorporating comprises incorporating in vivo. In some embodiments, the nucleic acid comprises a sequence encoding a membrane protein. In some embodiments, the nucleic acid comprises a sequence encoding a cytosolic protein. In some embodiments, the nucleic acid comprises a sequence encoding a recombinant protein. In some embodiments, the nucleic acid is an mRNA, wherein the mRNA is 50-20,000 nucleotides long.

In some embodiments, the nucleic acid comprises at least 1 modified nucleotide. In some embodiments, the nucleic acid comprises at least 10% modified nucleotides. In some embodiments, the nucleic acid comprises at least 20% modified nucleotides. In some embodiments, the nucleic acid comprises at least 30%, 40%, or 50% modified nucleotides. In some embodiments, less than 70% of the uridine residues in the nucleic acid are modified. In some embodiments, less than 50% of the uridine residues in the nucleic acid are modified. In some embodiments, the modified nucleotide is a pseudouridine, 1-methyl-pseudouridine or a 5-methoxyuridine that replaces a uridine.

In some embodiments, the nucleic acid is encapsulated in a lipid nanoparticle. In some embodiments, the lipid nanoparticle comprises a cationic lipid, and at least one of: a non-cationic lipid, a neutral lipid, cholesterol and a conjugated lipid. In some embodiments, the lipid nanoparticle comprises a cationic lipid, a non-cationic lipid and a conjugated lipid. In some embodiments, the conjugated lipid is a PEGylated lipid. In some embodiments, the PEG moiety is PEG-2000.

In some embodiments, following incorporation of the nucleic acid in a population of CD14+ cells ex vivo, at least 80% of cells in the population of CD14+ cells express the protein encoded by the nucleic acid at 24 hours. In some embodiments, following incorporation of the nucleic acid in a population of CD14+ cells ex vivo, at least 70% of the population express the protein encoded by the nucleic acid after 48 hours. In some embodiments, the protein is detectable in CD14+ cells up to 72 hours following incorporation of the nucleic acid. In some embodiments, the protein is detectable in CD14+ cells up to 96 hours following incorporation of the nucleic acid. In some embodiments, the protein is detectable in CD14+ cells up to 5 days following incorporation of the nucleic acid.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

An “antigen presenting cell” or “APC” as used herein includes professional antigen presenting cells (e.g., B lymphocytes, macrophages, monocytes, dendritic cells, Langerhans cells), as well as other antigen presenting cells (e.g., keratinocytes, endothelial cells, astrocytes, fibroblasts, oligodendrocytes, thymic epithelial cells, thyroid epithelial cells, glial cells (brain), pancreatic beta cells, and vascular endothelial cells). An APC can express Major Histocompatibility complex (MHC) molecules and can display antigens complexed with MHC on its surface which can be recognized by T cells and trigger T cell activation and an immune response. Professional antigen-presenting cells, notably dendritic cells, play a key role in stimulating naive T cells. Nonprofessional antigen-presenting cells, such as fibroblasts, may also contribute to this process. APCs can also cross-present peptide antigens by processing exogenous antigens and presenting the processed antigens on class I MHC molecules. Antigens that give rise to proteins that are recognized in association with class I MHC molecules are generally proteins that are produced within the cells, and these antigens are processed and associate with class I MHC molecules.

A “biological sample” can refer to any tissue, cell, fluid, or other material derived from an organism.

The term “epitope” can refer to any protein determinant, such as a sequence or structure or amino acid residues, capable of binding to an antibody or binding fragment thereof, a T cell receptor, and/or an antibody-like molecule. Epitopic determinants typically consist of chemically active surface groups of molecules such as amino acids or sugar side chains and generally have specific three dimensional structural characteristics as well as specific charge characteristics. A “T cell epitope” can refer to peptide or peptide-MHC complex recognized by a T cell receptor.

An engineered cell, such as an engineered myeloid cell, can refer to a cell that has at least one exogenous nucleic acid sequence in the cell, even if transiently expressed. Expressing an exogenous nucleic acid may be performed by various methods described elsewhere, and encompasses methods known in the art. The present disclosure relates to preparing and using engineered cells, for example, engineered myeloid cells, such as engineered phagocytic cells. The present disclosure relates to, inter alia, an engineered cell comprising an exogenous nucleic acid encoding, for example, a chimeric fusion protein (CFP).

The term “immune response” includes, but is not limited to, T cell mediated, NK cell mediated and/or B cell mediated immune responses. These responses may be influenced by modulation of T cell costimulation and NK cell costimulation. Exemplary immune responses include T cell responses, e.g., cytokine production, and cellular cytotoxicity. In addition, immune responses include immune responses that are indirectly affected by NK cell activation, B cell activation and/or T cell activation, e.g., antibody production (humoral responses) and activation of cytokine responsive cells, e.g., macrophages Immune responses include adaptive immune responses. The adaptive immune system can react to foreign molecular structures, such as antigens of an intruding organism. Unlike the innate immune system, the adaptive immune system is highly specific to a pathogen. Adaptive immunity can also provide long-lasting protection. Adaptive immune reactions include humoral immune reactions and cell-mediated immune reactions. In humoral immune reactions, antibodies secreted by B cells into bodily fluids bind to pathogen-derived antigens leading to elimination of the pathogen through a variety of mechanisms, e.g. complement-mediated lysis. In cell-mediated immune reactions, T cells capable of destroying other cells are activated. For example, if proteins associated with a disease are present in a cell, they can be fragmented proteolytically to peptides within the cell. Specific cell proteins can then attach themselves to the antigen or a peptide formed in this manner, and transport them to the surface of the cell, where they can be presented to molecular defense mechanisms, such as T cells. Cytotoxic T cells can recognize these antigens and kill cells that harbor these antigens.

A “ligand” can refer to a molecule which is capable of binding or forming a complex with another molecule, such as a receptor. A ligand can include, but is not limited to, a protein, a glycoprotein, a carbohydrate, a lipoprotein, a hormone, a fatty acid, a phospholipid, or any component that binds to a receptor. In some embodiments, a receptor has a specific ligand. In some embodiments, a receptor may have promiscuous binding to a ligand, in which case it can bind to several ligands that share at least a similarity in structural configuration, charge distribution or any other physicochemical characteristic. A ligand may be a biomolecule. A ligand may be an abiotic material. For example, a ligand may be a negative charged particle that is a ligand for scavenger receptor MARCO. For example, a ligand may be TiO₂, which is a ligand for the scavenger receptor SRA1.

The term “major histocompatibility complex (MHC)”, “MHC molecule”, or “MHC protein” refers to a protein capable of binding an antigenic peptide and present the antigenic peptide to T lymphocytes. Such antigenic peptides can represent T cell epitopes. The human MHC is also called the HLA complex. Thus, the terms “human leukocyte antigen (HLA)”, “HLA molecule” or “HLA protein” are used interchangeably with the terms “major histocompatibility complex (MHC)”, “MHC molecule”, and “MHC protein”. HLA proteins can be classified as HLA class I or HLA class II. The structures of the proteins of the two HLA classes are very similar; however, they have very different functions. Class I HLA proteins are present on the surface of almost all cells of the body, including most tumor cells. Class I HLA proteins are loaded with antigens that usually originate from endogenous proteins or from pathogens present inside cells, and are then presented to naïve or cytotoxic T-lymphocytes (CTLs). HLA class II proteins are present on antigen presenting cells (APCs), including but not limited to dendritic cells, B cells, and macrophages. They mainly present peptides which are processed from external antigen sources, e.g. outside of cells, to helper T cells.

In the HLA class II system, phagocytes such as macrophages and immature dendritic cells can take up entities by phagocytosis into phagosomes—though B cells exhibit the more general endocytosis into endosomes—which fuse with lysosomes whose acidic enzymes cleave the uptaken protein into many different peptides. Autophagy is another source of HLA class II peptides. The most studied subclass II HLA genes are: HLA-DPA1, HLA-DPB1, HLA-DQA1, HLA-DQB1, HLA-DRA, and HLA-DRB1.

Presentation of peptides by HLA class II molecules to CD4+ helper T cells can lead to immune responses to foreign antigens. Once activated, CD4+ T cells can promote B cell differentiation and antibody production, as well as CD8+ T cell (CTL) responses. CD4+ T cells can also secrete cytokines and chemokines that activate and induce differentiation of other immune cells. HLA class II molecules are typically heterodimers of α- and β-chains that interact to form a peptide-binding groove that is more open than class I peptide-binding grooves.

HLA alleles are typically expressed in codominant fashion. For example, each person carries 2 alleles of each of the 3 class I genes, (HLA-A, HLA-B and HLA-C) and so can express six different types of class II HLA. In the class II HLA locus, each person inherits a pair of HLA-DP genes (DPA1 and DPB1, which encode α and β chains), HLA-DQ (DQA1 and DQB1, for α and β chains), one gene HLA-DRa (DRA1), and one or more genes HLA-DR13 (DRB1 and DRB3, -4 or -5). HLA-DRB1, for example, has more than nearly 400 known alleles. That means that one heterozygous individual can inherit six or eight functioning class II HLA alleles: three or more from each parent. Thus, the HLA genes are highly polymorphic; many different alleles exist in the different individuals inside a population. Genes encoding HLA proteins have many possible variations, allowing each person's immune system to react to a wide range of foreign invaders. Some HLA genes have hundreds of identified versions (alleles), each of which is given a particular number. In some embodiments, the class I HLA alleles are HLA-A*02:01, HLA-B*14:02, HLA-A*23:01, HLA-E*01:01 (non-classical). In some embodiments, class II HLA alleles are HLA-DRB*01:01, HLA-DRB*01:02, HLA-DRB*11:01, HLA-DRB*15:01, and HLA-DRB*07:01.

A “myeloid cell” can refer broadly to cells of the myeloid lineage of the hematopoietic cell system, and can exclude, for example, the lymphocytic lineage. Myeloid cells comprise, for example, cells of the granulocyte lineage and monocyte lineages. Myeloid cells are differentiated from common progenitors derived from the hematopoietic stem cells in the bone marrow. Commitment to myeloid cell lineages may be governed by activation of distinct transcription factors, and accordingly myeloid cells may be characterized as cells having a level of plasticity, which may be described as the ability to further differentiate into terminal cell types based on extracellular and intracellular stimuli. Myeloid cells can be rapidly recruited into local tissues via various chemokine receptors on their surface. Myeloid cells are responsive to various cytokines and chemokines.

A myeloid cell, for example, may be a cell that originates in the bone marrow from a hematopoietic stem cell under the influence of one or more cytokines and chemokines, such as G-CSF, GM-CSF, Flt3L, CCL2, VEGF and S100A8/9. In some embodiments, the myeloid cell is a precursor cell. In some embodiments, the myeloid cell may be a cell having characteristics of a common myeloid progenitor, or a granulocyte progenitor, a myeloblast cell, or a monocyte-dendritic cell progenitor or a combination thereof. A myeloid can include a granulocyte or a monocyte or a precursor cell thereof. A myeloid can include an immature granulocyte, an immature monocyte, an immature macrophage, an immature neutrophil, and an immature dendritic cell. A myeloid can include a monocyte or a pre-monocytic cell or a monocyte precursor. In some cases, a myeloid cell as used herein may refer to a monocyte having an M0 phenotype, an M1 phenotype or an M2 phenotype. A myeloid can include a dendritic cell (DC), a mature DC, a monocyte derived DC, a plasmacytoid DC, a pre-dendritic cell, or a precursor of a DC. A myeloid can include a neutrophil, which may be a mature neutrophil, a neutrophil precursor, or a polymorphonucleocyte (PMN). A myeloid can include a macrophage, a monocyte-derived macrophage, a tissue macrophage, a macrophage of an M0, an M1 or an M2 phenotype. A myeloid can include a tumor infiltrating monocyte (TIM). A myeloid can include a tumor associated monocyte (TAM). A myeloid can include a myeloid derived suppressor cell (MDSC). A myeloid can include a tissue resident macrophage. A myeloid can include a tumor associated DC (TADC). Accordingly, a myeloid cell may express one or more cell surface markers, for example, CD11b, CD14, CD15, CD16, CD38, CCR5, CXCR1, CXCR2, CXCR4, CD66, Lox-1, CD11c, CD64, CD68, CD163, CCR2, CCR4, CCR7, CX3CR1, HLA-DR, CD1c, CD83, CD141, CD209, MHC-II, CD123, CD303, CD304, a SIGLEC family protein and a CLEC family protein. In some cases, a myeloid cell may be characterized by a high or a low expression of one or more of cell surface markers, for example, CD11b, CD14, CD15, CD16, CD66, Lox-1, CD11c, CD64, CD68, CD163, CCR2, CCR5, HLA-DR, CD1c, CD83, CD141, CD209, MHC-II, CD123, CD303, CD304 or a combination thereof.

“Phagocytosis” is used interchangeably with “engulfment” and can refer to a process by which a cell engulfs a particle, such as a cancer cell or an infected cell. This process can give rise to an internal compartment (phagosome) containing the particle. This process can be used to ingest and or remove a particle, such as a cancer cell or an infected cell from the body. A phagocytic receptor may be involved in the process of phagocytosis. The process of phagocytosis can be closely coupled with an immune response and antigen presentation. The processing of exogenous antigens follows their uptake into professional antigen presenting cells by some type of endocytic event. Phagocytosis can also facilitate antigen presentation. For example, antigens from phagocytosed cells or pathogens, including cancer antigens, can be processed and presented on the cell surface of APCs.

A “polypeptide” can refer to a molecule containing amino acids linked together via a peptide bond, such as a glycoprotein, a lipoprotein, a cellular protein or a membrane protein. A polypeptide may comprise one or more subunits of a protein. A polypeptide may be encoded by a nucleic acid. In some embodiments, polypeptide may comprise more than one peptide sequence in a single amino acid chain, which may be separated by a spacer, a linker or peptide cleavage sequence. A polypeptide may be a fused polypeptide. A polypeptide may comprise one or more domains, modules or moieties.

A “receptor” can refer to a chemical structure composed of a polypeptide, which transduces a signal, such as a polypeptide that transduces an extracellular signal to a cell. A receptor can serve to transmit information in a cell, a cell formation or an organism. A receptor comprises at least one receptor unit and can contain two or more receptor units, where each receptor unit comprises a protein molecule, e.g., a glycoprotein molecule. A receptor can contain a structure that binds to a ligand and can form a complex with the ligand. Signaling information can be transmitted by a conformational change of the receptor following binding with the ligand on the surface of a cell.

The term “antibody” refers to a class of proteins that are generally known as immunoglobulins, including, but not limited to IgG1, IgG2, IgG3, and IgG4, IgA (including IgA1 and IgA2), IgD, IgE, IgM, and IgY. The term “antibody” includes, but is not limited to, full length antibodies, single-chain antibodies, single domain antibodies (sdAb) and antigen-binding fragments thereof. Antigen-binding antibody fragments include, but are not limited to, Fab, Fab′ and F(ab′)2, Fd (consisting of V_{H} and C_{H}1), single-chain variable fragment (scFv), single-chain antibodies, disulfide-linked variable fragment (dsFv) and fragments comprising a V_{L} and/or a V_{H} domain. Antibodies can be from any animal origin. Antigen-binding antibody fragments, including single-chain antibodies, can comprise variable region(s) alone or in combination with one or more of a hinge region, a CH1 domain, a CH2 domain, and a CH3 domain. Also included are any combinations of variable region(s) and hinge region, CH1, CH2, and CH3 domains. Antibodies can be monoclonal, polyclonal, chimeric, humanized, and human monoclonal and polyclonal antibodies which, e.g., specifically bind an HLA-associated polypeptide or an HLA-peptide complex.

A nucleic acid as described herein can contain a nucleotide sequence that is not naturally occurring. An engineered nucleic acid may be synthesized in the laboratory. A nucleic acid may be prepared by using recombinant DNA technology, for example, enzymatic modification of DNA, such as enzymatic restriction digestion, ligation, and DNA cloning. A nucleic acid can be DNA, RNA, analogues thereof, or a combination thereof. A recombinant DNA may be transcribed ex vivo or in vitro, such as to generate a messenger RNA (mRNA). An mRNA as described herein may be isolated, purified and used to electroporate or transfect a cell. An mRNA as described herein can be an in vitro transcribed mRNA. A nucleic acid, such has an mRNA, described herein can be synthetic. A nucleic acid, such has an mRNA, described herein can be encoded by a vector. A nucleic acid, such has an mRNA, described herein may encode a protein or a polypeptide.

The process of introducing or incorporating a nucleic acid into a cell can be via electroporation, transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the introduction of small molecules such as DNA, RNA, or antibodies into eukaryotic cells. Transfection may also refer to the introduction of bacteriophage into bacterial cells. ‘Transduction’ is mostly used to describe the introduction of recombinant viral vector particles into target cells, while ‘infection’ refers to natural infections of humans or animals with wild-type viruses.

The term “vector”, can refer to a nucleic acid molecule capable of autonomous replication in a host cell, and which allow for cloning of nucleic acid molecules. As known to those skilled in the art, a vector includes, but is not limited to, a plasmid, cosmid, phagemid, viral vectors, phage vectors, yeast vectors, mammalian vectors and the like. For example, a vector for exogenous gene transformation may be a plasmid. In certain embodiments, a vector comprises a nucleic acid sequence containing an origin of replication and other elements necessary for replication and/or maintenance of the nucleic acid sequence in a host cell. In some embodiments, a vector or a plasmid provided herein is an expression vector. Expression vectors are capable of directing the expression of genes and/or nucleic acid sequence to which they are operatively linked. In some embodiments, an expression vector or plasmid is in the form of circular double stranded DNA molecules. A vector or plasmid may or may not be integrated into the genome of a host cell. In some embodiments, nucleic acid sequences of a plasmid are not integrated in a genome or chromosome of the host cell after introduction. For example, the plasmid may comprise elements for transient expression or stable expression of the nucleic acid sequences, e.g. genes or open reading frames harbored by the plasmid, in a host cell. In some embodiments, a vector is a transient expression vector. In some embodiments, a vector is a stably expressed vector that replicates autonomously in a host cell. In some embodiments, nucleic acid sequences of a plasmid are integrated into a genome or chromosome of a host cell upon introduction into the host cell. Expression vectors that can be used in the methods as disclosed herein include, but are not limited to, plasmids, episomes, bacterial artificial chromosomes, yeast artificial chromosomes, bacteriophages or viral vectors. A vector can be a DNA or RNA vector. In some embodiments, a vector provide herein is a RNA vector that is capable of integrating into a host cell's genome upon introduction into the host cell (e.g., via reverse transcription), for example, a retroviral vector or a lentiviral vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked.

The terms “spacer” or “linker” as used in reference to a fusion protein/polypeptide refers to a peptide sequence that joins two other peptide sequences of the fusion protein/polypeptide. In some embodiments, a linker or spacer has no specific biological activity other than to join or to preserve some minimum distance or other spatial relationship between the proteins or RNA sequences. In some embodiments, the constituent amino acids of a spacer can be selected to influence some property of the molecule such as the folding, flexibility, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. In some embodiments, a linker is used to separate two or more polypeptides, e.g. two antigenic peptides by a distance sufficient to ensure that each antigenic peptide properly folds. Exemplary peptide linker sequences adopt a flexible extended conformation and do not exhibit a propensity for developing an ordered secondary structure. Amino acids in flexible linker protein/peptode region may include Gly, Asn and Ser, or any permutation of amino acid sequences containing Gly, Asn and Ser. Other near neutral amino acids, such as Thr and Ala, also can be used in the linker sequence.

The terms “treat,” “treated,” “treating,” “treatment,” and the like are meant to refer to reducing, preventing, or ameliorating a disorder and/or symptoms associated therewith (e.g., a neoplasia or tumor or infectious agent or an autoimmune disease). “Treating” can refer to administration of the therapy to a subject after the onset, or suspected onset, of a disease (e.g., cancer or infection by an infectious agent or an autoimmune disease). “Treating” includes the concepts of “alleviating”, which can refer to lessening the frequency of occurrence or recurrence, or the severity, of any symptoms or other ill effects related to the disease and/or the side effects associated with therapy. The term “treating” also encompasses the concept of “managing” which refers to reducing the severity of a disease or disorder in a patient, e.g., extending the life or prolonging the survivability of a patient with the disease, or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. The term “prevent”, “preventing”, “prevention” and their grammatical equivalents as used herein, can refer to avoiding or delaying the onset of symptoms associated with a disease or condition in a subject that has not developed such symptoms at the time the administering of an agent or compound commences. In certain embodiments, treating a subject or a patient as described herein comprises administering a therapeutic composition, such as a drug, a metabolite, a preventive component, a nucleic acid, a peptide, or a protein that encodes or otherwise forms a drug, a metabolite or a preventive component. In some embodiments, treating comprises administering a cell or a population of cells to a subject in need thereof. In some embodiments, treating comprises administering to the subject one or more of engineered cells described herein, e.g. one or more engineered myeloid cells, such as phagocytic cells. Treating comprises treating a disease or a condition or a syndrome, which may be a pathological disease, condition or syndrome, or a latent disease, condition or syndrome. In some cases, treating, as used herein may comprise administering a therapeutic vaccine. In some embodiments, the engineered phagocytic cell is administered to a patient or a subject. In some embodiments, a cell administered to a human subject results in reduced immunogenicity. For example, an engineered phagocytic cell may lead to no or reduced graft versus host disease (GVHD) or fratricide effect. In some embodiments, an engineered cell administered to a human subject is immunocompatible to the subject (i.e. having a matching HLA subtype that is naturally expressed in the subject). Subject specific HLA alleles or HLA genotype of a subject can be determined by any method known in the art. In exemplary embodiments, the methods include determining polymorphic gene types that can comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

A “fragment” can refer to a portion of a protein or nucleic acid. In some embodiments, a fragment retains at least 50%, 75%, or 80%, or 90%, 95%, or even 99% of the biological activity of a reference protein or nucleic acid.

The terms “isolated,” “purified”, “biologically pure” and their grammatical equivalents refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of the present disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications can give rise to different isolated proteins, which can be separately purified.

The terms “neoplasia” or “cancer” refers to any disease that is caused by or results in inappropriately high levels of cell division, inappropriately low levels of apoptosis, or both. Glioblastoma is one non-limiting example of a neoplasia or cancer. The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell.

The term “vaccine” is to be understood as meaning a composition for generating immunity for the prophylaxis and/or treatment of diseases (e.g., neoplasia/tumor/infectious agents/autoimmune diseases). Accordingly, vaccines as used herein are medicaments which comprise engineered nucleic acids, or cells comprising and expressing a nucleic acid and are intended to be used in humans or animals for generating specific defense and protective substance by vaccination. A “vaccine composition” can include a pharmaceutically acceptable excipient, carrier or diluent. Aspects of the present disclosure relate to use of the technology in preparing a phagocytic cell-based vaccine.

The term “pharmaceutically acceptable” refers to approved or approvable by a regulatory agency of the Federal or a state government or listed in the U.S. Pharmacopeia or other generally recognized pharmacopeia for use in animals, including humans. A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Nucleic acid molecules useful in the methods of the disclosure include, but are not limited to, any nucleic acid molecule with activity or that encodes a polypeptide. Polynucleotides having substantial identity to an endogenous sequence are typically capable of hybridizing with at least one strand of a double-stranded nucleic acid molecule. “Hybridize” refers to when nucleic acid molecules pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration can ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide Stringent temperature conditions can ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an exemplary embodiment, hybridization can occur at 30° C. in 750 mM NaCl, 75 mM trisodium citrate, and 1% SDS. In another exemplary embodiment, hybridization can occur at 37° C. in 500 mM NaCl, 50 mM trisodium citrate, 1% SDS, 35% formamide, and 100 μg/ml denatured salmon sperm DNA (ssDNA). In another exemplary embodiment, hybridization can occur at 42° C. in 250 mM NaCl, 25 mM trisodium citrate, 1% SDS, 50% formamide, and 200 μg/ml ssDNA. Useful variations on these conditions will be readily apparent to those skilled in the art. For most applications, washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, or less than about 15 mM NaCl and 1.5 mM trisodium citrate. Stringent temperature conditions for the wash steps can include a temperature of at least about 25° C., of at least about 42° C., or at least about 68° C. In exemplary embodiments, wash steps can occur at 25° C. in 30 mM NaCl, 3 mM trisodium citrate, and 0.1% SDS. In other exemplary embodiments, wash steps can occur at 42° C. in 15 mM NaCl, 1.5 mM trisodium citrate, and 0.1% SDS. In another exemplary embodiment, wash steps can occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Substantially identical” refers to a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence can be at least 60%, 80% or 85%, 90%, 95%, 96%, 97%, 98%, or even 99% or more identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e-3 and e-m° indicating a closely related sequence. A “reference” is a standard of comparison. It will be understood that the numbering of the specific positions or residues in the respective sequences depends on the particular protein or polypeptide and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment to a reference sequence and determination of homologous residues.

The term “subject” or “patient” refers to an organism, such as an animal (e.g., a human) which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

The term “therapeutic effect” refers to some extent of relief of one or more of the symptoms of a disorder (e.g., a neoplasia, tumor, or infection by an infectious agent or an autoimmune disease) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required.

5′- and 3′-Untranslated Regions (UTRs), 5′-CAP and 3′ Poly a Tail of an mRNA

For generating an engineered polynucleotide construct for non-viral gene delivery and expression, factors that could potentially affect mRNA stability and translation efficiency were considered, for example, the features related to a 5′ untranslated region (5′ UTR), a 3′ UTR, and a polyadenylation (Poly A tail). The untranslated regions at the 5′- and the 3′-end flanking the coding sequence are critical for expression of the encoded polypeptide when introduced into a cell. Of these, the 5′-UTR is responsible for efficient assembly of the ribosome on the translational start codon. Some templates for in vitro transcription are known in the literature, which are utilized in a standardized manner and which have been modified in such a way that stabilized RNA transcripts are produced. Protocols currently described in the literature are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 Adenosine nucleotides. In some embodiments, Me engineered polynucleotide comprises an engineered untranslated region UTR).

Naturally occurring mRNAs contain a 5′ cap structure which helps stabilize the RNA and is fundamental to eukaryotic gene expression (Shuman, S. et al Mol. Microbiol. 1995, 17, 405-410). These mRNAs also contain a 3′ poly A tail. Both modifications stabilize the mRNA encoding the protein. The 5′-end modification can comprise a cap structure based on guanosine or purine that increase expression of products (e.g., protein) encoded by the RNA, lower immunogenicity and/or improve stability of the RNA. The 3′-end modifications can comprise modification of the cis-diol at the 3′ and 2′ positions of the terminal ribose of the RNA, for example by inserting a ring atom between these positions or replacing one or both hydroxyl groups with other substituents, to improve stability of the RNA (e.g., by slowing the rate of degradation). The 5′-cap structure found at the 5′ end of eukaryotic messenger RNAs (mRNAs) and many viral RNAs consists of a N7-methylguanosine nucleoside linked to the 5′-terminal nucleoside of the pre-mRNA via a 5′-5′ triphosphate linkage. This cap structure fulfills many roles that ultimately lead to mRNA translation. RNA capping is also important for other processes, such as RNA splicing and export from the nucleus and to avoid recognition of mRNA by the cellular innate immunity machinery (Daffis, S. et al Nature 2010, 468, 452-6; Zist, R. et al Nat. Immunol. 2011, 12, 137-43).

The 3′ UTR can determine the half-life of an mRNA in a cell. mRNA degradation is regulated through protein complexes binding to regions within the 3′ UTR and the poly A tail. For example, regulatory sequences in the 3′-UTR containing adenylate-uridylate-rich elements (AU-rich elements: AREs) can destabilize mRNAs. The most important regulatory elements involved are the poly A tail and other cis-elements but multiple and diversified regulatory mechanisms have been described (Oktaba et al., 2015; Yue et al., 2018).

### Enhancement of Expression of a Polypeptide Encoded by a Polynucleotide Using Non-Native or Synthetic 5′- and 3′ UTRs:

Provided herein are exemplary mRNA constructs that are engineered with non-native or synthetic 5′- and 3′-UTRs that greatly enhance the expression of a protein coding sequence comprised in a nucleic acid when introduced in a cell. In some embodiments the cell is a myeloid cell. In some embodiments, the 5′ UTR may be encoded within a plasmid in which the DNA sequence encoding an exogenous protein coding sequence is incorporated for delivery and expression. Likewise, in some embodiments, the 3′ UTR may also be encoded within a plasmid in which the DNA sequence encoding an exogenous protein coding sequence is incorporated for delivery and expression. In some embodiments, the nucleic acid is an IVT RNA. In some embodiments the 5′- and/or the 3′ UTR may be added to the mRNA comprising the sequence encoding an exogenous protein in vitro in an enzymatic reaction. A poly A tail can be added enzymatically in vitro to an RNA or can be an encoded poly A tail, such as by an IVT template. In some embodiments, enzymatically added poly A tails can be tailored to have a desired length.

A proper 5′-cap structure is important in the synthesis of functional messenger RNA. In some embodiments, the 5′-cap comprises a guanosine triphosphate arranged as GpppG at the 5′terminus of the nucleic acid.

In some embodiments, the mRNA comprises a 5′ 7-methylguanosine cap, m7-GpppG. A 5′ 7-methylguanosine cap can increase mRNA translational efficiency and prevents degradation of mRNA 5′-3′exonucleases. In some embodiments, the mRNA comprises “anti-reverse” cap analog (ARCA, m7,3′-0 GpppG).

In some embodiments, the guanosine cap is a Cap 0 structure.

In some embodiments, the guanosine cap is a Cap 1 structure.

In addition to its essential role of cap-dependent initiation of protein synthesis, the mRNA cap can also function as a protective group from 5′ to 3′ exonuclease cleavage and a unique identifier for recruiting protein factors for pre-mRNA splicing, polyadenylation and nuclear export. It acts as the anchor for the recruitment of initiation factors that initiate protein synthesis and the 5′ to 3′ looping of mRNA during translation. Three enzymatic activities are required to generate the Cap 0 structure, namely, RNA triphosphatase (TPase), RNA guanylyltransferase (GTase) and guanine-N7 methyltransferase (guanine-N7 MTase). Each of these enzyme activities carries out an essential step in the conversion of the 5′ triphosphate of nascent RNA to the Cap 0 structure. RNA TPase removes the ‘-phosphate from the 5’ triphosphate to generate 5′ diphosphate RNA. GTase transfers a GMP group from GTP to the 5′ diphosphate via a lysine-GMP covalent intermediate. The guanine-N7 MTase then adds a methyl group to the N7 amine of the guanine cap to form the cap 0 structure. For Cap 1 structure, m7G-specific 2′O methyltransferase (2′O MTase) methylates the +1 ribonucleotide at the 2′O position of the ribose to generate the cap 1 structure. The nuclear RNA capping enzyme interacts with the polymerase subunit of RNA polymerase II complex at phosphorylated Ser5 of the C-terminal heptad repeats. RNA guanine-N7 methyltransferase also interacts with the RNA polymerase II phosphorylated heptad repeats. In some embodiments, the cap is a G-quadruplex cap. In some embodiments, the 5′ Cap is added to the 5′ end of the mRNA by co-transcriptional capping, in which, a cap analog is introduced into the transcription reaction, along with the four standard nucleotide triphosphates, in an optimized ratio of cap analog to GTP 4:1. This allows initiation of the transcript with the cap structure in a large proportion of the synthesized RNA molecules. This approach produces a mixture of transcripts, of which ˜80% are capped, and the remainder have 5′triphosphate ends. In some embodiments, the cap analogs used in co-transcriptional RNA capping is the standard 7-methyl guanosine (m7G) cap analog.

In some embodiments, the cap analogs used in co-transcriptional RNA capping is the anti-reverse cap analog (ARCA), also known as 3′ 0-me 7-meGpppG cap analog. ARCA is methylated at the 3′ position of the m7G, preventing RNA elongation by phosphodiester bond formation at this position. Thus, transcripts synthesized using ARCA contain 5′-m7G cap structures in the correct orientation, with the 7-methylated G as the terminal residue. In contrast, the m7G cap analog can be incorporated in either the correct or the reverse orientation.

In one embodiment, the 5′ UTR comprises about 50 nucleotides. In some embodiments, the 5′ UTR comprises less than 50 nucleotides. In one embodiment, the 5′ UTR may comprise more than 50 nucleotides. In one embodiment, the 5′ UTR comprises at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 nucleotides.

In some embodiments, the 5′ UTR comprises is at least 45 nucleotides in length. In one embodiment, the 5′ UTR comprises a translation start site that is at least 15 nucleotides downstream of the 5′ end. In some embodiment, the 5′ UTR comprises is at least 45 nucleotides in length. In one embodiment, the 5′ UTR comprises a translation start site that is at least 20 nucleotides downstream of the 5′ end. In some embodiment, the 5′ UTR comprises is at least 45 nucleotides in length. In one embodiment, the 5′ UTR comprises a translation start site that is at least 25 nucleotides downstream of the 5′ end. In some embodiment, the 5′ UTR comprises is at least 45 nucleotides in length. In one embodiment, the 5′ UTR comprises a translation start site that is at least 30 nucleotides downstream of the 5′ end.

In some embodiments, the 5′ UTR comprises is at least 45 nucleotides in length. In one embodiment, the 5′ UTR comprises a single translational start site.

In some embodiments, addition of a poly-A tail at the end of transcription may be done enzymatically. For example, the poly A tailing may be performed using E. coli poly(A) polymerase. Alternatively, for example, the poly A tailing may be performed using Saccharomyces (yeast) poly(A) polymerase. The length of the poly A tail introduced can be optimized by titrating the reaction.

In some embodiments, the nucleic acid comprises about 100-250 poly adenosyl (poly (A)) residues.

In some embodiments, the poly A length is between 50-200 Adenosine nucleotide residues.

In some embodiments, the poly A length is at least about 120 Adenosine nucleotide residues.

In some embodiments, the poly A length is at least about 50, 60, 70, 80, 90, 100, 110, 120, 130, 140, 150, 160, 170, 180, 190, 200, 210, 220, 230, 240, or about 250 Adenosine residues.

In some embodiments the 5′ and 3′ UTRs for stabilizing the nucleic acid inside the cell and ensuring improved expression of the exogenous protein comprises any one of the sequences from SEQ ID NO: 46-59. In some embodiments, the cell is a myeloid cell. In some embodiments the cell is a myeloid precursor cell. In some embodiments, the cell is a CD14+ cell. In some embodiments the cell is a CD14+/CD16− cell.

In some embodiments, an mRNA having 50 nucleotides-20,000 nucleotides can be expressed in myeloid cells using the methods described herein. In some embodiments, the mRNA comprises a coding region of about 1000, 2000, 3000, 4000, 5000, 6000, 7000, 8000, 9000, 10,000, 11,000, 12,000, 13,000, 14,000 15,000, 16,000, 17,000, 18,000 19,000 or about 20,000 nucleotides.

In some embodiments, replacing the 5′ UTR and/or the 3′ UTR improves expression of the encoded protein or polypeptide by at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% at least 160%, at least 170%, at least 180%, at least 190%, at least 200% or more compared to standard UTRs.

In some embodiments, replacing the 5′ UTR and/or the 3′ UTR improves duration of expression of the encoded protein or polypeptide in the myeloid cell by at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% at least 160%, at least 170%, at least 180%, at least 190%, at least 200% or more compared to standard UTRs.

In some embodiments, the target cell is a mammalian cell. In some embodiments, the target cell is a human cell. In some embodiments, the target cell comprises a cell infected with a pathogen. In some embodiments, the target cell is a cancer cell. In some embodiments, the target cell is a cancer cell that is a lymphocyte. In some embodiments, the target cell is a cancer cell that is an ovarian cancer cell. In some embodiments, the target cell is a cancer cell that is a breast cell. In some embodiments, the target cell is a cancer cell that is a pancreatic cell. In some embodiments, the target cell is a cancer cell that is a glioblastoma cell.

In some embodiments, the nucleic acid is DNA. In some embodiments, the nucleic acid is RNA. In some embodiments, the nucleic acid is mRNA. In some embodiments, the nucleic acid is an unmodified mRNA. In some embodiments, the nucleic acid is a modified mRNA. In some embodiments, the nucleic acid is a circRNA. In some embodiments, the nucleic acid is a tRNA. In some embodiments, the nucleic acid is a microRNA. In some embodiments, the nucleic acid is an engineered nucleic acid. In some embodiments, the nucleic acid is a recombinant nucleic acid. In some embodiments, the nucleic acid is an in vitro transcribed nucleic acid.

Also provided herein is a vector comprising a nucleic acid sequence encoding a CFP described herein. In some embodiments, the vector is viral vector. In some embodiments, the viral vector is retroviral vector or a lentiviral vector. In some embodiments, the vector further comprises a promoter operably linked to at least one nucleic acid sequence encoding one or more polypeptides. In some embodiments, the vector is polycistronic. In some embodiments, each of the at least one nucleic acid sequence is operably linked to a separate promoter. In some embodiments, the vector further comprises one or more internal ribosome entry sites (IRESs). In some embodiments, the vector further comprises a 5′ UTR and/or a 3′ UTR flanking the at least one nucleic acid sequence encoding one or more polypeptides. In some embodiments, the vector further comprises one or more regulatory regions.

Also provided herein is a polypeptide encoded by the nucleic acid of a composition described herein.

Also provided herein is a cell comprising a composition described herein, a vector described herein or a polypeptide described herein. In some embodiments, the cell is a phagocytic cell. In some embodiments, the cell is a stem cell derived cell, a myeloid cell, a macrophage, a dendritic cell, a lymphocyte, a mast cell, a monocyte, a neutrophil, a microglia, or an astrocyte. In some embodiments, the cell is an autologous cell. In some embodiments, the cell is an allogeneic cell. In some embodiments, the cell is an M1 cell. In some embodiments, the cell is an M2 cell. In some embodiments, the cell is an M1 macrophage cell. In some embodiments, the cell is an M2 macrophage cell. In some embodiments, the cell is an M1 myeloid cell. In some embodiments, the cell is an M2 myeloid cell.

mRNA Nucleotide Modifications

In some aspects, the oligonucleotide described herein comprises at least one chemical modification. A chemical modification can be a substitution, insertion, deletion, chemical modification, physical modification, stabilization, purification, or any combination thereof. In some cases, a modification is a chemical modification. In one embodiments, the modification can be a phosphonate modification. In one aspect, the phosphonate modification is a phosphorothioate (PS Rp isomer). In one aspect, the phosphonate modification is a phosphorothioate (PS Sp isomer).

In some aspects, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, the chiral phosphate product is present in a diastereomeric excess of 95%. In some aspects, the chiral phosphate product is present in a diastereomeric excess of 96%. In some aspects, the chiral phosphate product is present in a diastereomeric excess of 97%. In some aspects, the chiral phosphate product is present in a diastereomeric excess of 98%. In some aspects, the chiral phosphate product is present in a diastereomeric excess of 99%. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In one embodiments, the modification can be a ribose modification. In one aspect, the ribose modification is a 2′-O-Methyl (2′-OME) modification. In one aspect, the ribose modification is 2′-O-Methoxyethyl (2′-O-MOE) modification. In one aspect, the ribose modification is 2′-deoxy-2′-fluoro (2′-F). In one aspect, the ribose modification is 2′-arabino fluoro (2′-Ara-F). In one aspect, the ribose modification is 2′-O-benzyl. In one aspect, the ribose modification is 2′-O-methyl-4-pyridine (2′-O—CH2Py(4)). In one aspect the ribose modification is a locked nucleic acid (LNA). In one aspect, the ribose modification can be a base modification. In one aspect the base modification is pseudouridine (ψ). In one aspect, the ribose modification is 2′-thiouridine (s2U). In one aspect, the ribose modification is N6′-methyladenosine (m⁶C). In one aspect, the ribose modification is 5′-methylcytidine (m⁵C). In one aspect, the ribose modification is 5′-fluoro-2′-dioxyuridine. In one aspect, the ribose modification is N-ethylpiperidine (7′-EAA triazole modified adenine. In one aspect, the ribose modification is N-ethylpiperidine 6′ triazole modified adenine. In one aspect, the ribose modification is 6-phenylpyrrolocytosine, In one aspect, the ribose modification is 2′-4′-difluorotoluyl ribonucleoside (rF). In one aspect, the ribose modification is 5′-nitroindole.

In some aspects, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the nucleic acids described herein. The nucleobase of the nucleotide can be independently selected from a purine, a pyrimidine, a purine or pyrimidine analog. In some aspects, the nucleobase can be naturally occurring or synthetic derivatives of a base.

In some aspects, the chemical modification described herein comprises modifying an uracil. In some aspects, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, l-taurinomethyl-4-thio-pseudouridine, 5-methyl-uridine, 1 methyl-pseudouridine, 5-methyl-2-thio-uridine, l-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine,1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some aspects, the chemical modification described herein comprises modifying a cytosine. In some aspects, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some aspects, the chemical modification described herein comprises modifying a adenine. In some aspects, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some aspects, the chemical modification described herein comprises modifying a guanine. In some aspects, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, l-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, 06-Methyl-2′-deoxyguanosine, 2′-F-ara-guanosine, and 2′-F-guanosine.

In some embodiments, the modification is in a terminal nucleotide.

In some embodiments, the modification is in an internal nucleotide.

In some embodiments, the mRNA is unmodified.

In some embodiments, the mRNA comprise 1, 2, or 3 or more modified nucleotides.

In some cases, a modification can be permanent. In other cases, a modification can be transient. In some cases, multiple modifications are made to the nucleic acid. The nucleic acid modification can alter physio-chemical properties of a nucleotide, such as their conformation, polarity, hydrophobicity, chemical reactivity, base-pairing interactions, or any combination thereof.

Chimeric Fusion Protein (CFP)—a Myeloid Cell with Target Specificity

Provided herein are compositions comprising a nucleic acid encoding a chimeric fusion protein (CFP), such as a phagocytic receptor (PR) fusion protein (PFP), a scavenger receptor (SR) fusion protein (SFP), an integrin receptor (IR) fusion protein (IFP) or a caspase-recruiting receptor (caspase-CAR) fusion protein. A CFP encoded by the nucleic acid can comprise an extracellular domain (ECD) comprising an antigen binding domain that binds to an antigen of a target cell. The extracellular domain can be fused to a hinge domain or an extracellular domain derived from a receptor, such as CD2, CD8, CD28, CD68, a phagocytic receptor, a scavenger receptor or an integrin receptor. The CFP encoded by the nucleic acid can further comprise a transmembrane domain, such as a transmembrane domain derived from CD2, CD8, CD28, CD68, a phagocytic receptor, a scavenger receptor or an integrin receptor. In some embodiments, a CFP encoded by the nucleic acid further comprises an intracellular domain comprising an intracellular signaling domain, such as an intracellular signaling domain derived from a phagocytic receptor, a scavenger receptor or an integrin receptor. For example, the intracellular domain can comprise one or more intracellular signaling domains derived from a phagocytic receptor, a scavenger receptor or an integrin receptor. For example, the intracellular domain can comprise one or more intracellular signaling domains that promote phagocytic activity, inflammatory response, nitric oxide production, integrin activation, enhanced effector cell migration (e.g., via chemokine receptor expression), antigen presentation, and/or enhanced cross presentation. In some embodiments, the CFP is a phagocytic receptor fusion protein (PFP). In some embodiments, the CFP is a phagocytic scavenger receptor fusion protein (PFP). In some embodiments, the CFP is an integrin receptor fusion protein (IFP). In some embodiments, the CFP is an inflammatory receptor fusion protein. In some embodiments, a CFP encoded by the nucleic acid further comprises an intracellular domain comprising a recruitment domain. For example, the intracellular domain can comprise one or more PI3K recruitment domains, caspase recruitment domains or caspase activation and recruitment domains (CARDs).

Provided herein is a composition comprising a nucleic acid encoding a CFP comprising a phagocytic or tethering receptor (PR) subunit (e.g., a phagocytic receptor fusion protein (PFP)) comprising: (i) a transmembrane domain, and (ii) an intracellular domain comprising a phagocytic receptor intracellular signaling domain; and an extracellular antigen binding domain specific to an antigen, e.g., an antigen of or presented on a target cell; wherein the transmembrane domain and the extracellular antigen binding domain are operatively linked such that antigen binding to the target by the extracellular antigen binding domain of the fused receptor activated in the intracellular signaling domain of the phagocytic receptor.

Provided herein is a composition comprising a nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP.

Provided herein is a composition comprising a nucleic acid sequence encoding a CFP comprising a phagocytic or tethering receptor (PR) subunit (e.g., a phagocytic receptor fusion protein (PFP)) comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein upon binding of the CFP to the antigen of the target cell, the killing or phagocytosis activity of a myeloid cell, such as a neutrophil, monocyte, myeloid dendritic cell (mDC), mast cell or macrophage expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold-fold, 17-fold, 18-fold, 19-fold, 20-fold, 25-fold, 30-fold, 40-fold, 50-fold, 75-fold, or 100-fold compared to a cell not expressing the CFP.

In one aspect, provided herein is a pharmaceutical composition comprising: (a) a myeloid cell, such as a neutrophil, monocyte, myeloid dendritic cell (mDC), mast cell or macrophage cell comprising a polynucleic acid, wherein the polynucleic acid comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP and exhibits at least a 1.1-fold increase in phagocytosis of a target cell expressing CD5 compared to a myeloid cell not expressing the CFP. In some embodiments, the CD5 binding domain is a CD5 binding protein that comprises an antigen binding fragment of an antibody, an Fab fragment, an scFv domain or an sdAb domain. In some embodiments, the CD5 binding domain comprises an scFv comprising: (i) a variable heavy chain (V_{H}) sequence of SEQ ID NO: 1 or with at least 90% sequence identity to SEQ ID NO: 1; and (ii) a variable light chain (V_{L}) sequence of SEQ ID NO: 2 or with at least 70%, 75%, 80%, 85%, 90%, 91%, 92%, 93%, 94%, 95%, 96%, 97%, 98% or 99% sequence identity to SEQ ID NO: 2. In some embodiments, the CFP further comprises an intracellular domain, wherein the intracellular domain comprises one or more intracellular signaling domains, and wherein a wild-type protein comprising the intracellular domain does not comprise the extracellular domain.

In some embodiments, the extracellular domain further comprises a hinge domain derived from CD8, wherein the hinge domain is operatively linked to the transmembrane domain and the anti-CD5 binding domain.

In some embodiments, the transmembrane domain comprises a CD8 transmembrane domain.

In some embodiments, the CFP comprises one or more intracellular signaling domains that comprise a phagocytic signaling domain. In some embodiments, the phagocytosis signaling domain comprises an intracellular signaling domain derived from a receptor other than Megf10, MerTk, FcRα, and Bail. In some embodiments, the phagocytosis signaling domain comprises an intracellular signaling domain derived from a receptor other than Megf10, MerTk, an FcR, and Bail. In some embodiments, the phagocytosis signaling domain comprises an intracellular signaling domain derived from a receptor other than CD3. In some embodiments, the phagocytosis signaling domain comprises an intracellular signaling domain derived from FcRγ, FcRα or FGRE. In some embodiments, the phagocytosis signaling domain comprises an intracellular signaling domain derived from CD3ζ. In some embodiments, the one or more intracellular signaling domains further comprises a proinflammatory signaling domain. In some embodiments, the proinflammatory signaling domain comprises a PI3-kinase (PI3K) recruitment domain.

In some embodiments, the CFP comprises: (a) an extracellular domain comprising: (i) a scFv that specifically binds CD5, and (ii) a hinge domain derived from CD8; a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRα, FcRγ or FcRε, and (ii) a second intracellular signaling domain: (A) comprising a PI3K recruitment domain, or (B) derived from CD40. In some embodiments, the CFP comprises as an alternative (c) to the above: an intracellular domain comprising at least two intracellular signaling domains, wherein the at least two intracellular signaling domains comprise: (i) a first intracellular signaling domain derived from a phagocytic receptor intracellular domain, and (ii) a second intracellular signaling domain derived from a scavenger receptor phagocytic receptor intracellular domain comprising: (A) comprising a PI3K recruitment domain, or (B) derived from CD40. Table 2 enlists an exemplary set of various domains and combinations thereof, that were used to design the CFPs described herein. In some embodiments, the CFP comprises and intracellular signaling domain derived from an intracellular signaling domain of an innate immune receptor.

In some embodiments, the polynucleic acid is an mRNA. In some embodiments, the polynucleic acid is a circRNA. In some embodiments, the polynucleic acid is a viral vector. In some embodiments, the polynucleic acid is delivered via a viral vector.

In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In one aspect, provided herein is a method of treating cancer in a human subject in need thereof comprising administering a pharmaceutical composition to the human subject, the pharmaceutical composition comprising: (a) a myeloid cell comprising a polynucleic acid sequence, wherein the polynucleic acid sequence comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP.

In some embodiments, upon binding of the CFP to CD5 expressed by a target cancer cell of the subject killing or phagocytosis activity of the myeloid cell is increased by greater than 20% compared to a myeloid cell not expressing the CFP. In some embodiments, growth of a tumor is inhibited in the human subject.

In some embodiments, the cancer is a CD5+ cancer. In some embodiments, the cancer is leukemia, T cell lymphoma, or B cell lymphoma.

In some embodiments, the anti-CD5 binding domain is a CD5 binding protein that comprises an antigen binding fragment of an antibody, an scFv domain, an Fab fragment, or an sdAb domain. In some embodiments, the anti-CD5 binding domain is a protein or fragment thereof that binds to CD5, such as a ligand of CD5 (e.g., a natural ligand of CD5).

In some embodiments, the CFP further comprises an intracellular domain, wherein the intracellular domain comprises one or more intracellular signaling domains, wherein the one or more intracellular signaling domains comprises a phagocytosis signaling domain and wherein a wild-type protein comprising the intracellular domain does not comprise the extracellular domain.

In some embodiments, the phagocytosis signaling domain comprises an intracellular signaling domain derived from a receptor other than Megf10, MerTk, FcRα and Bail. In some embodiments, the phagocytosis signaling domain comprises an intracellular signaling domain derived from FcRγ, FcRα or FcRε.

In some embodiments, the one or more intracellular signaling domains further comprises a proinflammatory signaling domain. In some embodiments, the proinflammatory signaling domain comprises a PI3-kinase (PI3K) recruitment domain. In some embodiments, the transmembrane domain comprises a CD8 transmembrane domain. In some embodiments, the extracellular domain comprises a hinge domain derived from CD8, a hinge domain derived from CD28 or at least a portion of an extracellular domain from CD68.

In some embodiments, the CFP comprises: (a) an extracellular domain comprising: (i) a scFv that specifically binds CD5, and (ii) a hinge domain derived from CD8, a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRγ or FORE, and (ii) a second intracellular signaling domain that: (A) comprises a PI3K recruitment domain, or (B) is derived from CD40. In some embodiments, the nucleic acid is mRNA or circRNA. In some embodiments, the myeloid cell is a CD14+ cell, a CD14+/CD16− cell, a CD14+/CD16+ cell, a CD14−/CD16+ cell, CD14−/CD16− cell, a dendritic cell, an M0 macrophage, an M2 macrophage, an M1 macrophage or a mosaic myeloid cell/macrophage/dendritic cell.

In some embodiments, the method further comprises administering an additional therapeutic agent selected from the group consisting of a CD47 agonist, an agent that inhibits Rac, an agent that inhibits Cdc42, an agent that inhibits a GTPase, an agent that promotes F-actin disassembly, an agent that promotes PI3K recruitment to the PFP, an agent that promotes PI3K activity, an agent that promotes production of phosphatidylinositol 3,4,5-trisphosphate, an agent that promotes ARHGAP12 activity, an agent that promotes ARHGAP25 activity, an agent that promotes SH3BP1 activity, an agent that promotes sequestration of lymphocytes in primary and/or secondary lymphoid organs, an agent that increases concentration of naïve T cells and central memory T cells in secondary lymphoid organs, and any combination thereof.

In some embodiments, the myeloid cell further comprises: (a) an endogenous peptide or protein that dimerizes with the CFP, (b) a non-endogenous peptide or protein that dimerizes with the CFP; and/or (c) a second recombinant polynucleic acid sequence, wherein the second recombinant polynucleic acid sequence comprises a sequence encoding a peptide or protein that interacts with the CFP; wherein the dimerization or the interaction potentiates phagocytosis by the myeloid cell expressing the CFP as compared to a myeloid cell that does not express the CFP.

In some embodiments, the myeloid cell exhibits (i) an increase in effector activity, cross-presentation, respiratory burst, ROS production, iNOS production, inflammatory mediators, extra-cellular vesicle production, phosphatidylinositol 3,4,5-trisphosphate production, trogocytosis with the target cell expressing the antigen, resistance to CD47 mediated inhibition of phagocytosis, resistance to LILRB1 mediated inhibition of phagocytosis, or any combination thereof; and/or (ii) an increase in expression of a IL-1, IL3, IL-6, IL-10, IL-12, IL-13, IL-23, TNFα, a TNF family of cytokines, CCL2, CXCL9, CXCL10, CXCL11, IL-18, IL-23, IL-27, CSF, MCSF, GMCSF, IL-17, IP-10, RANTES, an interferon, MHC class I protein, MHC class II protein, CD40, CD48, CD58, CD80, CD86, CD112, CD155, a TRAIL/TNF Family death receptor, TGFβ, B7-DC, B7-H2, LIGHT, HVEM, TL1A, 41BBL, OX40L, GITRL, CD30L, TIM1, TIM4, SLAM, PDL1, an MMP (e.g., MMP2, MMP7 and MMP9) or any combination thereof.

In some embodiments, the intracellular signaling domain is derived from a phagocytic or tethering receptor or wherein the intracellular signaling domain comprises a phagocytosis activation domain. In some embodiments, the intracellular signaling domain is derived from a receptor other than a phagocytic receptor selected from Megf10, MerTk, FcR-alpha, or Bail. In some embodiments, the intracellular signaling domain is derived from a protein, such as receptor (e.g., a phagocytic receptor), selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, a complement receptor, CR3, CR4, Tim-1, Tim-4 and CD169. In some embodiments, the intracellular signaling domain comprises a pro-inflammatory signaling domain. In some embodiments, the intracellular signaling domain comprises a pro-inflammatory signaling domain that is not a PI3K recruitment domain.

In some embodiments, the intracellular signaling domain is derived from an ITAM domain containing receptor.

Provided herein is a composition comprising a nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a phagocytic receptor other than a phagocytic receptor selected from Megf10, MerTk, FcRα, or Bail.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold-fold, 17-fold, 18-fold, 19-fold, 20-fold, 25-fold, 30-fold, 40-fold, 50-fold, 75-fold, or 100-fold compared to a cell not expressing the CFP.

In some embodiments, the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3ζ, CR3, CR4, Tim-1, Tim-4 and CD169. In some embodiments, the intracellular signaling domain comprises a pro-inflammatory signaling domain.

Provided herein is a composition comprising a nucleic acid encoding a CFP, such as a phagocytic or tethering receptor (PR) fusion protein (PFP), comprising: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked; and wherein the intracellular signaling domain is derived from a receptor, such as a phagocytic receptor, selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fcα receptor I, CR1, CD35, CD3, CR3, CR4, Tim-1, Tim-4 and CD169.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the intracellular signaling domain is derived from a phagocytic receptor other than a phagocytic receptor selected from Megf10, MerTk, FcRα, or Bail. In some embodiments, the intracellular signaling domain comprises a pro-inflammatory signaling domain. In some embodiments, the intracellular signaling domain comprises a PI3K recruitment domain, such as a PI3K recruitment domain derived from CD19. In some embodiments, the intracellular signaling domain comprises a pro-inflammatory signaling domain that is not a PI3K recruitment domain.

Provided herein is a composition of an engineered CFP, such as a phagocytic receptor fusion protein, that may be expressed in a cell, such as a myeloid cell, such as to generate an engineered myeloid cell that can target a target cell, such as a diseased cell.

A target cell is, for example, a cancer cell. In some embodiments, the engineered myeloid cell, after engulfment of a cancer cell may present a cancer antigen on its cell surface to activate a T cell. An “antigen” is a molecule capable of stimulating an immune response. Antigens recognized by T cells, whether helper T lymphocytes (T helper (TH) cells) or cytotoxic T lymphocytes (CTLs), are not recognized as intact proteins, but rather as small peptides that associate with MHC proteins (such as class I or class II MHC proteins) on the surface of cells. During the course of a naturally occurring immune response, antigens that are recognized in association with class II MHC molecules on antigen presenting cells (APCs) are acquired from outside the cell, internalized, and processed into small peptides that associate with the class II MHC molecules.

In some embodiments, upon binding of the CFP to the antigen of the target cell, the killing activity of a cell expressing the CFP is increased by at least greater than 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 100%, 150%, 200%, 250%, 300%, 350%, 400%, 450%, 500%, 550%, 600%, 650%, 700%, 750%, 800%, 850%, 900%, 950%, or 1000% compared to a cell not expressing the CFP. In some embodiments, the CFP functionally incorporates into a cell membrane of a cell when the CFP is expressed in the cell. In some embodiments, upon binding of the CFP to the antigen of the target cell, the killing activity of a cell expressing the CFP is increased by at least 1.1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold-fold, 17-fold, 18-fold, 19-fold, 20-fold, 25-fold, 30-fold, 40-fold, 50-fold, 75-fold, or 100-fold compared to a cell not expressing the CFP.

In some embodiments, the target cell expressing the antigen is a cancer cell. In some embodiments, the target cell expressing the antigen is at least 0.8 microns in diameter.

In some embodiments, a cell expressing the CFP exhibits an increase in phagocytosis of a target cell expressing the antigen compared to a cell not expressing the CFP. In some embodiments, a cell expressing the CFP exhibits at least a 1.1-fold increase in phagocytosis of a target cell expressing the antigen compared to a cell not expressing the CFP. In some embodiments, a cell expressing the CFP exhibits at least a 2-fold, 3-fold, 4-fold, 5-fold, 6-fold, 7-fold, 8-fold, 9-fold, 10-fold, 20-fold, 30-fold or 50-fold increase in phagocytosis of a target cell expressing the antigen compared to a cell not expressing the CFP. In some embodiments, a cell expressing the CFP exhibits an increase in production of a cytokine compared to a cell not expressing the CFP. In some embodiments, the cytokine is selected from the group consisting of IL-1, IL3, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, an interferon and combinations thereof. In some embodiments, a cell expressing the CFP exhibits an increase in effector activity compared to a cell not expressing the CFP. In some embodiments, a cell expressing the CFP exhibits an increase in cross-presentation compared to a cell not expressing the CFP. In some embodiments, a cell expressing the CFP exhibits an increase in expression of an MHC class II protein compared to a cell not expressing the CFP. In some embodiments, a cell expressing the CFP exhibits an increase in expression of CD80 compared to a cell not expressing the CFP. In some embodiments, a cell expressing the CFP exhibits an increase in expression of CD86 compared to a cell not expressing the CFP. In some embodiments, a cell expressing the CFP exhibits an increase in expression of MHC class I protein compared to a cell not expressing the CFP. In some embodiments, a cell expressing the CFP exhibits an increase in expression of TRAIL/TNF Family death receptors compared to a cell not expressing the CFP. In some embodiments, a cell expressing the CFP exhibits an increase in expression of B7-H2 compared to a cell not expressing the CFP. In some embodiments, a cell expressing the CFP exhibits an increase in expression of LIGHT compared to a cell not expressing the CFP. In some embodiments, a cell expressing the CFP exhibits an increase in expression of HVEM compared to a cell not expressing the CFP. In some embodiments, a cell expressing the CFP exhibits an increase in expression of CD40 compared to a cell not expressing the CFP. In some embodiments, a cell expressing the CFP exhibits an increase in expression of TL1A compared to a cell not expressing the CFP. In some embodiments, a cell expressing the CFP exhibits an increase in expression of 41BBL compared to a cell not expressing the CFP. In some embodiments, a cell expressing the CFP exhibits an increase in expression of OX40L compared to a cell not expressing the CFP. In some embodiments, a cell expressing the CFP exhibits an increase in expression of GITRL death receptors compared to a cell not expressing the CFP. In some embodiments, a cell expressing the CFP exhibits an increase in expression of CD30L compared to a cell not expressing the CFP. In some embodiments, a cell expressing the CFP exhibits an increase in expression of TIM4 compared to a cell not expressing the CFP. In some embodiments, a cell expressing the CFP exhibits an increase in expression of TIM1 ligand compared to a cell not expressing the CFP. In some embodiments, a cell expressing the CFP exhibits an increase in expression of SLAM compared to a cell not expressing the CFP. In some embodiments, a cell expressing the CFP exhibits an increase in expression of CD48 compared to a cell not expressing the CFP. In some embodiments, a cell expressing the CFP exhibits an increase in expression of CD58 compared to a cell not expressing the CFP. In some embodiments, a cell expressing the CFP exhibits an increase in expression of CD155 compared to a cell not expressing the CFP. In some embodiments, a cell expressing the CFP exhibits an increase in expression of CD112 compared to a cell not expressing the CFP. In some embodiments, a cell expressing the CFP exhibits an increase in expression of PDL1 compared to a cell not expressing the CFP. In some embodiments, a cell expressing the CFP exhibits an increase in expression of B7-DC compared to a cell not expressing the CFP. In some embodiments, a cell expressing the CFP exhibits an increase in respiratory burst compared to a cell not expressing the CFP. In some embodiments, a cell expressing the CFP exhibits an increase in ROS production compared to a cell not expressing the CFP. In some embodiments, a cell expressing the CFP exhibits an increase in iNOS production compared to a cell not expressing the CFP. In some embodiments, a cell expressing the CFP exhibits an increase in iNOS production compared to a cell not expressing the CFP. In some embodiments, a cell expressing the CFP exhibits an increase in extra-cellular vesicle production compared to a cell not expressing the CFP. In some embodiments, a cell expressing the CFP exhibits an increase in trogocytosis with a target cell expressing the antigen compared to a cell not expressing the CFP. In some embodiments, a cell expressing the CFP exhibits an increase in resistance to CD47 mediated inhibition of phagocytosis compared to a cell not expressing the CFP. In some embodiments, a cell expressing the CFP exhibits an increase in resistance to LILRB1 mediated inhibition of phagocytosis compared to a cell not expressing the CFP. In some embodiments, a cell expressing the CFP exhibits an increase in phosphatidylinositol 3,4,5-trisphosphate production.

In some embodiments, the extracellular domain of a CFP comprises an Ig binding domain. In some embodiments, the extracellular domain comprises an IgA, IgD, IgE, IgG, IgM, FcRγI, FcRγIIA, FcRγIIB, FcRγIIC, FcRγIIIA, FcRγIIIB, FcRn, TRIM21, FcRL5 binding domain. In some embodiments, the extracellular domain of a CFP comprises an FcR extracellular domain. In some embodiments, the extracellular domain of a CFP comprises an FcRα, FcRβ, FcRε or FcRγ extracellular domain. In some embodiments, the extracellular domain comprises an FcRα (FCAR) extracellular domain. In some embodiments, the extracellular domain comprises an FcRβ extracellular domain. In some embodiments, the extracellular domain comprises an FCER1A extracellular domain. In some embodiments, the extracellular domain comprises an FDGR1A, FCGR2A, FCGR2B, FCGR2C, FCGR3A, or FCGR3B extracellular domain. In some embodiments, the extracellular domain comprises an integrin domain or an integrin receptor domain. In some embodiments, the extracellular domain comprises one or more integrin α1, α2, αIIb, α3, α4, α5, α6, α7, α8, α9, α10, α11, αD, αE, αL, αM, αV, αX, β1, β2, β3, β4, β5, β6, β7, or β8 domains.

In some embodiments, the CFP further comprises an extracellular domain operatively linked to the transmembrane domain and the extracellular antigen binding domain. In some embodiments, the extracellular domain further comprises an extracellular domain of a receptor, a hinge, a spacer and/or a linker. In some embodiments, the extracellular domain comprises an extracellular portion of a phagocytic receptor. In some embodiments, the extracellular portion of the CFP is derived from the same receptor as the receptor from which the intracellular signaling domain is derived. In some embodiments, the extracellular domain comprises an extracellular domain of a scavenger receptor. In some embodiments, the extracellular domain comprises an immunoglobulin domain. In some embodiments, the immunoglobulin domain comprises an extracellular domain of an immunoglobulin or an immunoglobulin hinge region. In some embodiments, the extracellular domain comprises a phagocytic engulfment domain. In some embodiments, the extracellular domain comprises a structure capable of multimeric assembly. In some embodiments, the extracellular domain comprises a scaffold for multimerization. In some embodiments, the extracellular domain is at least 10, 20, 30, 40, 50, 60, 70, 80, 90, 100, 150, 200, 300, 300, 400, or 500 amino acids in length. In some embodiments, the extracellular domain is at most 500, 400, 300, 200, or 100 amino acids in length. In some embodiments, the extracellular antigen binding domain specifically binds to the antigen of a target cell. In some embodiments, the extracellular antigen binding domain comprises an antibody domain. In some embodiments, the extracellular antigen binding domain comprises a receptor domain, antibody domain, wherein the antibody domain comprises a functional antibody fragment, a single chain variable fragment (scFv), an Fab, a single-domain antibody (sdAb), a nanobody, a V_{H} domain, a V_{L} domain, a VNAR domain, a V_{HH} domain, a bispecific antibody, a diabody, or a functional fragment or a combination thereof. In some embodiments, the extracellular antigen binding domain comprises a ligand, an extracellular domain of a receptor or an adaptor. In some embodiments, the extracellular antigen binding domain comprises a single extracellular antigen binding domain that is specific for a single antigen. In some embodiments, the extracellular antigen binding domain comprises at least two extracellular antigen binding domains, wherein each of the at least two extracellular antigen binding domains is specific for a different antigen.

In some embodiments, the antigen is a cancer associated antigen, a lineage associated antigen, a pathogenic antigen or an autoimmune antigen. In some embodiments, the antigen comprises a viral antigen. In some embodiments, the antigen is a T lymphocyte antigen. In some embodiments, the antigen is an extracellular antigen. In some embodiments, the antigen is an intracellular antigen. In some embodiments, the antigen is selected from the group consisting of an antigen from Thymidine Kinase (TK1), Hypoxanthine-Guanine Phosphoribosyltransferase (HPRT), Receptor Tyrosine Kinase-Like Orphan Receptor 1 (ROR1), Mucin-1, Mucin-16 (MUC16), MUC1, Epidermal Growth Factor Receptor vIII (EGFRvIII), Mesothelin, Human Epidermal Growth Factor Receptor 2 (HER2), EBNA-1, LEMD1, Phosphatidyl Serine, Carcinoembryonic Antigen (CEA), B-Cell Maturation Antigen (BCMA), Glypican 3 (GPC3), Follicular Stimulating Hormone receptor, Fibroblast Activation Protein (FAP), Erythropoietin-Producing Hepatocellular Carcinoma A2 (EphA2), EphB2, a Natural Killer Group 2D (NKG2D) ligand, Disialoganglioside 2 (GD2), CD2, CD3, CD4, CD5, CD7, CD8, CD19, CD20, CD22, CD24, CD30, CD33, CD38, CD44v6, CD45, CD56CD79b, CD97, CD117, CD123, CD133, CD138, CD171, CD179a, CD213A2, CD248, CD276, PSCA, CS-1, CLECL1, GD3, PSMA, FLT3, TAG72, EPCAM, IL-1, an integrin receptor, PRSS21, VEGFR2, PDGFRβ, SSEA-4, EGFR, NCAM, prostase, PAP, ELF2M, GM3, TEM7R, CLDN6, TSHR, GPRC5D, ALK, Dsg1, Dsg3, IGLL1 and combinations thereof. In some embodiments, the antigen is an antigen of a protein selected from the group consisting of CD2, CD3, CD4, CD5, CD7, CCR4, CD8, CD30, CD45, and CD56. In some embodiments, the antigen is an ovarian cancer antigen or a T lymphoma antigen. In some embodiments, the antigen is an antigen of an integrin receptor. In some embodiments, the antigen is an antigen of an integrin receptor or integrin selected from the group consisting of α1, α2, αIIb, α3, α4, α5, α6, α7, α8, α9, α10, α11, αD, αE, αL, αM, αV, αX, β1, β2, β3, β4, β5, β6, β7, and β8. In some embodiment, the antigen is an antigen of an integrin receptor ligand. In some embodiments, the antigen is an antigen of fibronectin, vitronectin, collagen, or laminin. In some embodiments, the antigen binding domain can bind to two or more different antigens.

In some embodiments, the antigen binding domain comprises an autoantigen or fragment thereof, such as Dsg1 or Dsg3. In some embodiments, the extracellular antigen binding domain comprises a receptor domain or an antibody domain wherein the antibody domain binds to an auto antigen, such as Dsg1 or Dsg3.

In some embodiments, the transmembrane domain and the extracellular antigen binding domain are operatively linked through a linker. In some embodiments, the transmembrane domain and the extracellular antigen binding domain are operatively linked through a linker such as a hinge region of CD8α, IgG1 or IgG4.

In some embodiments, the extracellular domain comprises a multimerization scaffold.

In some embodiments, the transmembrane domain comprises a CD8 transmembrane domain. In some embodiments, the transmembrane domain comprises a CD28 transmembrane domain. In some embodiments, the transmembrane domain comprises a CD68 transmembrane domain. In some embodiments, the transmembrane domain comprises a CD2 transmembrane domain. In some embodiments, the transmembrane domain comprises an FcR transmembrane domain. In some embodiments, the transmembrane domain comprises an FcRγ transmembrane domain. In some embodiments, the transmembrane domain comprises an FcRα transmembrane domain. In some embodiments, the transmembrane domain comprises an FcRβ transmembrane domain. In some embodiments, the transmembrane domain comprises an FcRε transmembrane domain. In some embodiments, the transmembrane domain comprises a transmembrane domain from a syntaxin, such as syntaxin 3 or syntaxin 4 or syntaxin 5. In some embodiments, the transmembrane domain oligomerizes with a transmembrane domain of an endogenous receptor when the CFP is expressed in a cell. In some embodiments, the transmembrane domain oligomerizes with a transmembrane domain of an exogenous receptor when the CFP is expressed in a cell. In some embodiments, the transmembrane domain dimerizes with a transmembrane domain of an endogenous receptor when the CFP is expressed in a cell. In some embodiments, the transmembrane domain dimerizes with a transmembrane domain of an exogenous receptor when the CFP is expressed in a cell. In some embodiments, the transmembrane domain is derived from a protein that is different than the protein from which the intracellular signaling domain is derived. In some embodiments, the transmembrane domain is derived from a protein that is different than the protein from which the extracellular domain is derived. In some embodiments, the transmembrane domain comprises a transmembrane domain of a phagocytic receptor. In some embodiments, the transmembrane domain and the extracellular domain are derived from the same protein. In some embodiments, the transmembrane domain is derived from the same protein as the intracellular signaling domain. In some embodiments, the nucleic acid encodes a DAP12 recruitment domain. In some embodiments, the transmembrane domain comprises a transmembrane domain that oligomerizes with DAP12.

In some embodiments, the transmembrane domain is at least 12, 13, 14, 15, 16, 17, 18, 19, 20, 21, 22, 23, 24, 25, 26, 27, 28, 29, 30, 31 or 32 amino acids in length. In some embodiments, the transmembrane domain is at most 12, 13, 14, 15, 16, 17, 18, 19, 20, 21, 22, 23, 24, 25, 26, 27, 28, 29, 30, 31 or 32 amino acids in length.

In some embodiments, the intracellular signaling domain comprises an intracellular signaling domain derived from a phagocytic receptor. In some embodiments, the intracellular signaling domain comprises an intracellular signaling domain derived from a phagocytic receptor other than a phagocytic receptor selected from Megf10, MerTk, FcRα, or Bail. In some embodiments, the intracellular signaling domain comprises an intracellular signaling domain derived from a phagocytic receptor selected from the group consisting of TNFR1, MDA5, CD40, lectin, dectin 1, CD206, scavenger receptor A1 (SRA1), MARCO, CD36, CD163, MSR1, SCARA3, COLEC12, SCARA5, SCARB1, SCARB2, CD68, OLR1, SCARF1, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), CD64, CD32a, CD16a, CD89, Fc-alpha receptor I, CR1, CD35, CD3, CR3, CR4, Tim-1, Tim-4 and CD169. In some embodiments, the intracellular signaling domain comprises a PI3K recruitment domain. In some embodiments, the intracellular signaling domain comprises an intracellular signaling domain derived from a scavenger receptor. In some embodiments, the intracellular domain comprises a CD47 inhibition domain. In some embodiments, the intracellular domain comprises a Rac inhibition domain, a Cdc42 inhibition domain or a GTPase inhibition domain. In some embodiments, the Rac inhibition domain, the Cdc42 inhibition domain or the GTPase inhibition domain inhibits Rac, Cdc42 or GTPase at a phagocytic cup of a cell expressing the PFP. In some embodiments, the intracellular domain comprises an F-actin disassembly activation domain, a ARHGAP12 activation domain, a ARHGAP25 activation domain or a SH3BP1 activation domain. In some embodiments, the intracellular domain comprises a phosphatase inhibition domain. In some embodiments, the intracellular domain comprises an ARP2/3 inhibition domain. In some embodiments, the intracellular domain comprises at least one ITAM domain. In some embodiments, the intracellular domain comprises at least 1, 2, 3, 4, 5, 6, 7, 8, 9, 10, or more ITAM domains. In some embodiments, the intracellular domain comprises at least one ITAM domain select from an ITAM domain of CD3 zeta, CD3 epsilon, CD3 gamma, CD3 delta, Fc epsilon receptor 1 chain, Fc epsilon receptor 2 chain, Fc gamma receptor 1 chain, Fc gamma receptor 2a chain, Fc gamma receptor 2b 1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some embodiments, the at least one ITAM domain comprises a Src-family kinase phosphorylation site. In some embodiments, the at least one ITAM domain comprises a Syk recruitment domain. In some embodiments, the intracellular domain comprises an F-actin depolymerization activation domain. In some embodiments, the intracellular domain lacks enzymatic activity.

In some embodiments, the intracellular domain does not comprise a domain derived from a CD3 zeta intracellular domain. In some embodiments, the intracellular domain does not comprise a domain derived from a MerTK intracellular domain. In some embodiments, the intracellular domain does not comprise a domain derived from a TLR4 intracellular domain. In some embodiments, the intracellular domain comprises a CD47 inhibition domain. In some embodiments, the intracellular signaling domain comprises a domain that activates integrin, such as the intracellular region of PSGL-1. In some embodiments, the intracellular signaling domain comprises a domain that activates Rap1 GTPase, such as that from EPAC and C3G. In some embodiments, the intracellular signaling domain is derived from paxillin. In some embodiments, the intracellular signaling domain activates focal adhesion kinase. In some embodiments, the intracellular signaling domain is derived from a single phagocytic receptor. In some embodiments, the intracellular signaling domain is derived from a single scavenger receptor. In some embodiments, the intracellular domain comprises a phagocytosis enhancing domain.

In some embodiments, the intracellular domain comprises a pro-inflammatory signaling domain. In some embodiments, the pro-inflammatory signaling domain comprises a kinase activation domain or a kinase binding domain. In some embodiments, the pro-inflammatory signaling domain comprises an IL-1 signaling cascade activation domain. In some embodiments, the pro-inflammatory signaling domain comprises an intracellular signaling domain derived from TLR3, TLR4, TLR7, TLR 9, TRIF, RIG-1, MYD88, MAL, IRAK1, MDA-5, an IFN-receptor, STING, a NLRP family member, NLRP1-14, NOD1, NOD2, Pyrin, AIM2, NLRC4, FCGR3A, FCERIG, CD40, Tank1-binding kinase (TBK), a caspase domain, a procaspase binding domain or any combination thereof.

In some embodiments, the intracellular domain comprises a signaling domain, such as an intracellular signaling domain, derived from a connexin (Cx) protein. For example, the intracellular domain can comprise a signaling domain, such as an intracellular signaling domain, derived from Cx43, Cx46, Cx37, Cx40, Cx33, Cx50, Cx59, Cx62, Cx32, Cx26, Cx31, Cx30.3, Cx31.1, Cx30, Cx25, Cx45, Cx47, Cx31.3, Cx36, Cx31.9, Cx39, Cx40.1 or Cx23. For example, the intracellular domain can comprise a signaling domain, such as an intracellular signaling domain, derived from Cx43.

In some embodiments, the intracellular domain comprises a signaling domain, such as an intracellular signaling domain, derived from a SIGLEC protein. For example, the intracellular domain can comprise a signaling domain, such as an intracellular signaling domain, derived from Siglec-1 (Sialoadhesin), Siglec-2 (CD22), Siglec-3 (CD33), Siglec-4 (MAG), Siglec-5, Siglec-6, Siglec-7, Siglec-8, Siglec-9, Siglec-10, Siglec-11, Siglec-12, Siglec-13, Siglec-14, Siglec-15, Siglec-16 or Siglec-17.

In some embodiments, the intracellular domain comprises a signaling domain, such as an intracellular signaling domain, derived from a C-type lectin protein. For example, the intracellular domain can comprise a signaling domain, such as an intracellular signaling domain, derived from a mannose receptor protein. For example, the intracellular domain can comprise a signaling domain, such as an intracellular signaling domain, derived from an asialoglycoprotein receptor protein. For example, the intracellular domain can comprise a signaling domain, such as an intracellular signaling domain, derived from macrophage galactose-type lectin (MGL), DC-SIGN (CLEC4L), Langerin (CLEC4K), Myeloid DAP12 associating lectin (MDL)-1 (CLEC5A), a DC associated C type lectin 1 (Dectin1) subfamily protein, dectin 1/CLEC7A, DNGR1/CLEC9A, Myeloid C type lectin like receptor (MICL) (CLEC12A), CLEC2 (CLEC1B), CLEC12B, a DC immunoreceptor (DCIR) subfamily protein, DCIR/CLEC4A, Dectin 2/CLEC6A, Blood DC antigen 2 (BDCA2) (CLEC4C), Mincle (macrophage inducible C type lectin) (CLEC4E), a NOD-like receptor protein, NOD-like receptor MHC Class II transactivator (CIITA), IPAF, BIRC1, a RIG-I-like receptor (RLR) protein, RIG-I, MDA5, LGP2, NAIP5/Birc1e, a NLRP protein, NLRP1, NLRP2, NLRP3, NLRP4, NLRP5, NLRP6, NLRP7, NLRP89, NLRP9, NLRP10, NLRP11, NLRP12, NLRP13, NLRP14, a NLR protein, NOD1 or NOD2, or any combination thereof.

In some embodiments, the intracellular domain comprises a signaling domain, such as an intracellular signaling domain, derived from a cell adhesion molecule. For example, the intracellular domain can comprise a signaling domain, such as an intracellular signaling domain, derived from an IgCAMs, a cadherin, an integrin, a C-type of lectin-like domains protein (CTLD) and/or a proteoglycan molecule. For example, the intracellular domain can comprise a signaling domain, such as an intracellular signaling domain, derived from an E-cadherin, a P-cadherin, a N-cadherin, a R-cadherin, a B-cadherin, a T-cadherin, or a M-cadherin. For example, the intracellular domain can comprise a signaling domain, such as an intracellular signaling domain, derived from a selectin, such as an E-selectin, an L-selectin or a P-selectin.

In some embodiments, the CFP does not comprise a full length intracellular signaling domain. In some embodiments, the intracellular domain is at least 5, 10, 20, 30, 40, 50, 60, 70, 80, 90, 100, 150, 200, 300, 300, 400, or 500 amino acids in length. In some embodiments, the intracellular domain is at most 10, 20, 30, 40, 50, 60, 70, 80, 90, 100, 150, 200, 300, 300, 400, or 500 amino acids in length.

In some embodiments, the nucleic acid encodes an FcRα chain extracellular domain, an FcRα chain transmembrane domain and/or an FcRα chain intracellular domain. In some embodiments, the nucleic acid encodes an FcRβ chain extracellular domain, an FcRβ chain transmembrane domain and/or an FcRβ chain intracellular domain. In some embodiments, the FcRα chain or the FcRβ chain forms a complex with FcRγ when expressed in a cell. In some embodiments, the FcRα chain or FcRβ chain forms a complex with endogenous FcRγ when expressed in a cell. In some embodiments, the FcRα chain or the FcRβ chain does not incorporate into a cell membrane of a cell that does not express FcRγ. In some embodiments, the CFP does not comprise an FcRα chain intracellular signaling domain. In some embodiments, the CFP does not comprise an FcRβ chain intracellular signaling domain. In some embodiments, the nucleic acid encodes a TREM extracellular domain, a TREM transmembrane domain and/or a TREM intracellular domain. In some embodiments, the TREM is TREM1, TREM 2 or TREM 3.

In some embodiments, the nucleic acid comprises a sequence encoding a pro-inflammatory polypeptide. In some embodiments, the composition further comprises a proinflammatory nucleotide or a nucleotide in the nucleic acid, for example, an ATP, ADP, UTP, UDP, and/or UDP-glucose.

In some embodiments, the composition further comprises a pro-inflammatory polypeptide. In some embodiments, the pro-inflammatory polypeptide is a chemokine, cytokine. In some embodiments, the chemokine is selected from the group consisting of IL-1, IL3, IL5, IL-6, il8, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon. In some embodiments, the cytokine is selected from the group consisting of IL-1, IL3, IL5, IL-6, IL-12, IL-13, IL-23, TNF, CCL2, CXCL9, CXCL10, CXCL11, IL-18, IL-23, IL-27, CSF, MCSF, GMCSF, IL17, IP-10, RANTES, and interferon.

In some embodiments, the myeloid cells are specifically targeted for delivery. Myeloid cells can be targeted using specialized biodegradable polymers, such as PLGA (poly(lactic-co-glycolic) acid and/or polyvinyl alcohol (PVA). In some embodiments, one or more compounds can be selectively incorporated in such polymeric structures to affect the myeloid cell function. In some embodiments, the targeting structures are multilayered, e.g., of one or more PLGA and one or more PVA layers. In some embodiments, the targeting structures are assembled in an order for a layered activity. In some embodiments, the targeted polymeric structures are organized in specific shaped components, such as labile structures that can adhere to a myeloid cell surface and deliver one or more components such as growth factors and cytokines, such as to maintain the myeloid cell in a microenvironment that endows a specific polarization. In some embodiments, the polymeric structures are such that they are not phagocytosed by the myeloid cell, but they can remain adhered on the surface. In some embodiments the one or more growth factors may be M1 polarization factors, such as a cytokine. In some embodiments the one or more growth factors may be an M2 polarization factor, such as a cytokine. In some embodiments, the one or more growth factors may be a macrophage activating cytokine, such as IFNγ. In some embodiments the polymeric structures are capable of sustained release of the one or more growth factors in an in vivo environment, such as in a solid tumor.

In some embodiments, the nucleic acid comprises a sequence encoding a homeostatic regulator of inflammation. In some embodiments, the homeostatic regulator of inflammation is a sequence in an untranslated region (UTR) of an mRNA. In some embodiments, the sequence in the UTR is a sequence that binds to an RNA binding protein. In some embodiments, translation is inhibited or prevented upon binding of the RNA binding protein to the sequence in an untranslated region (UTR). In some embodiments, the sequence in the UTR comprises a consensus sequence of WWWU(AUUUA)UUUW, wherein W is A or U. In some embodiments, the nucleic acid is expressed on a bicistronic vector.

### Engineered Myelod Cells “Targeted” to Attack Diseased Cells

The present disclosure involves compositions and methods for preparing targeted killer myeloid cells; by leveraging the innate functional role in immune defense, ranging from properties related to detecting foreign bodies, particles, diseased cells, cellular debris, inflammatory signal, chemoattract; activating endogenous DAMP and PAMP signaling pathways; trigger myelopoiesis, extravasation; chemotaxis; phagocytes; pinocytosis; recruitment; engulfment; scavenging; activating intracellular oxidative burst and lysis or killing of pathogens, detecting, engulfing and killing diseased or damaged cells; removing unwanted cellular, tissue or acellular debris in vivo; antigen presentation and role in activating innate immunity; activating and modulating an immune response cascade; activating T cell repertoire; autophagy; inflammatory and non-inflammatory apoptosis; pyroptosis, immune editing to response to stress and restoration of tissue homeostasis. In one aspect, provided herein are methods and compositions to augment one or more functions of a myeloid cell for use in a therapeutic application, the one or more functions may be one or more of: detecting foreign bodies, particles, diseased cells, cellular debris, inflammatory signal, chemoattract; activating endogenous DAMP and PAMP signaling pathways; trigger myelopoiesis, extravasation; chemotaxis; phagocytosis; pinocytosis; recruitment; trogocytosis; engulfment; scavenging; activating intracellular oxidative burst and intracellular lysis or killing of pathogens, detecting, engulfing and killing diseased or damaged cells; removing unwanted cellular, tissue or acellular debris in vivo; antigen presentation and role in activating innate immunity; activating and modulating an immune response cascade; activating T cell repertoire; autophagy; inflammatory and non-inflammatory apoptosis; pyroptosis, immune editing to response to stress and restoration of tissue homeostasis. In one embodiment, the compositions and methods are also directed to augmenting the targeting, and killing function of certain myeloid cells, by genetic modification of these cells. The compositions and methods described herein are directed to creating engineered myeloid cells, wherein the engineered myeloid cells comprise at least one genetic modification, and can be directed to recognize and induce effector functions against a pathogen, a diseased cell, such as a tumor or cancer cell, such that the engineered myeloid cell is capable of recognizing, targeting, phagocytosing, killing and/or eliminating the pathogen or the diseased cell or the cancer cell, and additionally, may activate a specific immune response cascade following the phagocytosis, killing and/or eliminating the pathogen or the diseased cell.

Myeloid cells appear to be the most abundant cells in a tumor. Myeloid cells are also capable of recognizing a tumor cell over a healthy normal cell of the body and mount an immune response to a tumor cell of the body. As sentinels of innate immune response, myeloid cells are able to sense non-self or aberrant cell types and clear them via a process called phagocytosis. This can be directed to a therapeutic advantage in driving myeloid cell mediated phagocytosis and lysis of tumor cells. However, these naturally occurring tumor-infiltrating myeloid cells (TIMs) may be subjected to influence of the tumor microenvironment (TME). TIMs constitute a heterogeneous population of cells. Many TIMs originate from circulating monocytes and granulocytes, which in turn stem from bone marrow-derived hematopoietic stem cells. However, in the presence of persistent stimulation by tumor-derived factors the monocyte and granulocyte progenitors divert from their intrinsic pathway of terminal differentiation into mature macrophages, DCs or granulocytes, and may become tumor promoting myeloid cell types. Differentiation into pathological, alternatively activated immature myeloid cells is favored. These immature myeloid cells include tumor-associated DCs (TADCs), tumor-associated neutrophils (TANs), myeloid-derived suppressor cells (MDSCs), and tumor-associated macrophages (TAMs). Alternative to this emergency myelopoiesis, TAMs may also originate from tissue-resident macrophages, which in turn can be of embryonic or monocytic origin. These tissue-resident macrophages undergo changes in phenotype and function during carcinogenesis, and proliferation may help to maintain TAMs derived from tissue-resident macrophages. A tumor microenvironment may drive a tumor infiltrating myeloid cell to become myeloid derived suppressor cells and acquire the ability to suppress T cells. As a result, innovative methods are necessary to create therapeutically effective TIMs that can infiltrate a tumor, and can target tumor cells for phagocytic uptake and killing.

In one aspect, provided herein are engineered myeloid cells that are capable of targeting specific target cells, for example, tumor cells or pathogenic cells. In some embodiments, engineered myeloid cells provided herein are potent in infiltrating, targeting, and killing tumor cells. An engineered myeloid/phagocytic cell described herein is designed to comprise a nucleic acid, which encodes one or more proteins that help target the phagocytic cell to a target cell, for example a tumor cell or a cancer cell. In one embodiment, the engineered myeloid cell is capable of readily infiltrating a tumor. In one embodiment, the engineered myeloid cell has high specificity for the target cell, with none or negligible cross-reactivity to a non-tumor, non-diseased cell of the subject while in circulation. In one embodiment, the engineered myeloid/phagocytic cell described herein is designed to comprise a nucleic acid, which will help the cell to overcome/bypass the TME influence and mount a potent anti-tumor response. In one embodiment, the engineered myeloid/phagocytic cell described herein is designed to comprise a nucleic acid, which augments phagocytosis of the target cell. In another embodiment, the engineered myeloid/phagocytic cell described herein is designed to comprise a nucleic acid, which augments reduce or eliminate trogocytosis and/or enhance phagocytic lysis or of the target cell.

Accordingly, in some embodiments, the compositions disclosed herein comprise a myeloid cell, comprising a nucleic acid encoding a chimeric receptor fusion protein (CFP), for example, a phagocytic receptor (PR) fusion protein (PFP). The nucleic acid can comprise a sequence encoding a PR subunit comprising: (i) a transmembrane domain, and (ii) an intracellular domain comprising a PR intracellular signaling domain; and an extracellular antigen binding domain specific to an antigen of a target cell; wherein the transmembrane domain and the extracellular antigen binding domain are operatively linked; wherein the PR intracellular signaling domain is derived from a receptor with a signal transduction domain. The nucleic acid further encodes for one or more polypeptides that constitute one or more plasma membrane receptors that helps engage the phagocytic cell to the target cell, and enhance its phagocytic activity.

In some embodiments, the myeloid cell described herein comprises one or more recombinant proteins comprising a chimeric receptor, wherein the chimeric receptor is capable of responding to a first phagocytic signal directed to a target cell, which may be a diseased cell, a tumor cell or a pathogen, and a second signal, which is an inflammatory signal, that augments the phagocytic and killing response to target initiated by the first signal. In some embodiments, the recombinant proteins may comprise one or more of the amino acid sequences depicted in the SEQ ID NOs 1-33 in Table 1A. In some embodiments, the myeloid cell comprises a nucleic acid comprising a sequence that encodes one or more amino acid sequences selected from SEQ ID NOs. 1-33 in Table 1A.

TABLE 1A
Sequences of chimeric PFPs and domains thereof

| SEQ ID | | |
|---|---|---|
| NO | PFP/Domain | Sequence |
| 1 | Anti-CD5 heavy | EIQLVQSGGGLVKPGGSVRISCAASGYTFTNYGMNWVRQAP |
| | chain variable | GKGLEWMGWINTHTGEPTYADSFKGRFTFSLDDSKNTAYLQ |
| | domain | INSLRAEDTAVYFCTRRGYDWYFDVWGQGTTVTV |
| 2 | Anti-CD5 light | DIQMTQSPSSLSASVGDRVTITCRASQDINSYLSWFQQKPGK |
| | chain variable | APKTLIYRANRLESGVPSRFSGSGSGTDYTLTISSLQYEDFGIY |
| | domain | YCQQYDESPWTFGGGTKLEIK |
| 3 | Anti-CD5 scFv | EIQLVQSGGGLVKPGGSVRISCAASGYTFTNYGMNWVRQAP |
| | | GKGLEWMGWINTHTGEPTYADSFKGRFTFSLDDSKNTAYLQ |
| | | INSLRAEDTAVYFCTRRGYDWYFDVWGQGTTVTVSSGGGGS |
| | | GGGGSGGGGSDIQMTQSPSSLSASVGDRVTITCRASQDINSYL |
| | | SWFQQKPGKAPKTLIYRANRLESGVPSRFSGSGSGTDYTLTIS |
| | | SLQYEDFGIYYCQQYDESPWTFGGGTKLEIK |
| 4 | FcRγ-chain | LYCRRLKIQVRKAAITSYEKSDGVYTGLSTRNQETYETLKHE |
| | intracellular | KPPQ |
| | signaling domain | |
| 5 | FcRγ-chain | LYCRLKIQVRKAAITSYEKSDGVYTGLSTRNQETYETLKHEK |
| | intracellular | PPQ |
| | signaling domain | |
| 6 | FcRγ-chain | RLKIQVRKAAITSYEKSDGVYTGLSTRNQETYETLKHEKPPQ |
| | intracellular | |
| | signaling domain | |
| 7 | FcRγ-chain | RLKIQVRKAAITSYEKSDGVYTGLSTRNQETYETLKHEKPPQ |
| | intracellular | |
| | signaling domain | |
| 8 | PI3K recruitment | YEDMRGILYAAPQLRSIRGQPGPNHEEDADSYENM |
| | domain | |
| 5 | CD40 intracellular | KKVAKKPTNKAPHPKQEPQEINFPDDLPGSNTAAPVQETLHG |
| | domain | CQPVTQEDGKESRISVQERQ |
| 9 | CD8α chain | IYIWAPLAGTCGVLLLSLVIT |
| | transmembrane | |
| | domain | |
| 10 | CD8α chain | IYIWAPLAGTCGVLLLSLVITLYC |
| | transmembrane | |
| | domain | |
| 11 | CD8α chain hinge | ALSNSIMYFSHFVPVFLPAKPTTTPAPRPPTPAPTIASQPLSLRP |
| | domain | EACRPAAGGAVHTRGLD |
| 12 | Anti-HER2 heavy | DIQMTQSPSSLSASVGDRVTITCRASQDVNTAVAWYQQKPG |
| | chain variable | KAPKLLIYSASFLYSGVPSRFSGSRSGTDFTLTISSLQPEDFAT |
| | domain | YYCQQHYTTPPTFGQGTKVEIKRTGSTSGSGKPGSGEGSEVQ |
| | | LVE |
| 13 | Anti-HER2 light | LVQPGGSLRLSCAASGFNIKDTYIHWVRQAPGKGLEWVARI |
| | chain variable | YPTNGYTRYADSVKGRFTISADTSKNTAYLQMNSLRAEDTA |
| | domain | VYYCSRWGGDGFYAMDVWGQGTLVTV |
| 14 | Anti-HER2 scFv | DIQMTQSPSSLSASVGDRVTITCRASQDVNTAVAWYQQKPG |
| | | KAPKLLIYSASFLYSGVPSRFSGSRSGTDFTLTISSLQPEDFAT |
| | | YYCQQHYTTPPTFGQGTKVEIKRTGSTSGSGKPGSGEGSEVQ |
| | | LVESSGGGGSGGGGSGGGGSLVQPGGSLRLSCAASGFNIKDT |
| | | YIHWVRQAPGKGLEWVARIYPTNGYTRYADSVKGRFTISAD |
| | | TSKNTAYLQMNSLRAEDTAVYYCSRWGGDGFYAMDVWGQ |
| | | GTLVTV |
| 15 | GMCSF Signal | MWLQSLLLLGTVACSIS |
| | peptide | |
| 16 | CD28 | FWVLVVVGGVLACYSLLVTVAFIIFWV |
| | transmembrane | |
| | domain | |
| 17 | CD2 | IYLIIGICGGGSLLMVFVALLVFYIT |
| | Transmembrane | |
| | domain | |
| 18 | CD68 | ILLPLIIGLILLGLLALVLIAFCII |
| | transmembrane | |
| | domain | |
| 19 | TNFR1 | QRWKSKLYSIVCGKSTPEKEGELEGTTTKPLAPNPSFSPTPGF |
| | intracellular | TPTLGFSPVPSSTFTSSSTYTPGDCPNFAAPRREVAPPYQGAD |
| | domain | PILATALASDPIPNPLQKWEDSAHKPQSLDTDDPATLYAVVE |
| | | NVPPLRWKEFVRRLGLSDHEIDRLELQNGRCLREAQYSMLA |
| | | TWRRRTPRREATLELLGRVLRDMDLLGCLEDIEEALCGPAAL |
| | | PPAPSLLR |
| 20 | TNFR2 | PLCLQREAKVPHLPADKARGTQGPEQQHLLITAPSSSSSSLES |
| | intracellular | SASALDRRAPTRNQPQAPGVEASGAGEARASTGSSDSSPGGH |
| | domain | GTQVNVTCIVNVCSSSDHSSQCSSQASSTMGDTDSSPSESPKD |
| | | EQVPFSKEECAFRSQLETPETLLGSTEEKPLPLGVPDAGMKPS |
| 21 | MDA5 intracellular | MSNGYSTDENFRYLISCFRARVKMYIQVEPVLDYLTFLPAEV |
| | domain | KEQIQRTVATSGNMQAVELLLSTLEKGVWHLGWTREFVEAL |
| | | RRTGSPLAARYMNPELTDLPSPSFENAHDEYLQLLNLLQPTL |
| | | VDKLLVRDVLDKCMEEELLTIEDRNRIAAAENNGNESGVRE |
| | | LLKRIVQKENWFSAFLNVLRQTGNNELVQELTGSDCSESNAE |
| | | IEN |
| 22 | CD8α chain hinge | ALSNSIMYFSHFVPVFLPAKPTTTPAPRPPTPAPTIASQPLSLRP |
| | domain + | EACRPAAGGAVHTRGLDIYIWAPLAGTCGVLLLSLVITLYC |
| | transmembrane | |
| | domain | |
| 23 | CD8α chain hinge | ALSNSIMYFSHFVPVFLPAKPTTTPAPRPPTPAPTIASQPLSLRP |
| | domain + | EACRPAAGGAVHTRGLDIYIWAPLAGTCGVLLLSLVIT |
| | transmembrane | |
| | domain | |
| 24 | CD5-FcRγ-PI3K | MWLQSLLLLGTVACSISEIQLVQSGGGLVKPGGSVRISCAAS |
| | | GYTFTNYGMNWVRQAPGKGLEWMGWINTHTGEPTYADSF |
| | | KGRFTFSLDDSKNTAYLQINSLRAEDTAVYFCTRRGYDWYF |
| | | DVWGQGTTVTVSSGGGGSGGGGSGGGGSDIQMTQSPSSLSA |
| | | SVGDRVTITCRASQDINSYLSWFQQKPGKAPKTLIYRANRLE |
| | | SGVPSRFSGSGSGTDYTLTISSLQYEDFGIYYCQQYDESPWTF |
| | | GGGTKLEIKSGGGGSGALSNSIMYFSHFVPVFLPAKPTTTPAP |
| | | RPPTPAPTIASQPLSLRPEACRPAAGGAVHTRGLDIYIWAPLA |
| | | GTCGVLLLSLVITLYCRRLKIQVRKAAITSYEKSDGVYTGLST |
| | | RNQETYETLKHEKPPQGSGSYEDMRGILYAAPQLRSIRGQPG |
| | | PNHEEDADSYENM |
| 25 | HER2-FcRγ-PI3K | MWLQSLLLLGTVACSISDIQMTQSPSSLSASVGDRVTITCRAS |
| | | QDVNTAVAWYQQKPGKAPKLLIYSASFLYSGVPSRFSGSRSG |
| | | TDFTLTISSLQPEDFATYYCQQHYTTPPTFGQGTKVEIKRTGS |
| | | TSGSGKPGSGEGSEVQLVESGGGLVQPGGSLRLSCAASGFNI |
| | | KDTYIHWVRQAPGKGLEWVARIYPTNGYTRYADSVKGRFTI |
| | | SADTSKNTAYLQMNSLRAEDTAVYYCSRWGGDGFYAMDV |
| | | WGQGTLVTVSSSGGGGSGALSNSIMYFSHFVPVFLPAKPTTTP |
| | | APRPPTPAPTIASQPLSLRPEACRPAAGGAVHTRGLDIYIWAP |
| | | LAGTCGVLLLSLVITLYCRRLKIQVRKAAITSYEKSDGVYTG |
| | | LSTRNQETYETLKHEKPPQGSGSYEDMRGILYAAPQLRSIRG |
| | | QPGPNHEEDADSYENM |
| 26 | CD5-FcRγ-CD40 | MWLQSLLLLGTVACSISEIQLVQSGGGLVKPGGSVRISCAAS |
| | | GYTFTNYGMNWVRQAPGKGLEWMGWINTHTGEPTYADSF |
| | | KGRFTFSLDDSKNTAYLQINSLRAEDTAVYFCTRRGYDWYF |
| | | DVWGQGTTVTVSSGGGGSGGGGSGGGGSDIQMTQSPSSLSA |
| | | SVGDRVTITCRASQDINSYLSWFQQKPGKAPKTLIYRANRLE |
| | | SGVPSRFSGSGSGTDYTLTISSLQYEDFGIYYCQQYDESPWTF |
| | | GGGTKLEIKSGGGGSGALSNSIMYFSHFVPVFLPAKPTTTPAP |
| | | RPPTPAPTIASQPLSLRPEACRPAAGGAVHTRGLDIYIWAPLA |
| | | GTCGVLLLSLVITLYCRLKIQVRKAAITSYEKSDGVYTGLSTR |
| | | NQETYETLKHEKPPQKKVAKKPTNKAPHPKQEPQEINFPDDL |
| | | PGSNTAAPVQETLHGCQPVTQEDGKESRISVQERQ |
| 27 | CD5-FcRγ-MDA5 | MWLQSLLLLGTVACSISEIQLVQSGGGLVKPGGSVRISCAAS |
| | | GYTFTNYGMNWVRQAPGKGLEWMGWINTHTGEPTYADSF |
| | | KGRFTFSLDDSKNTAYLQINSLRAEDTAVYFCTRRGYDWYF |
| | | DVWGQGTTVTVSSGGGGSGGGGSGGGGSDIQMTQSPSSLSA |
| | | SVGDRVTITCRASQDINSYLSWFQQKPGKAPKTLIYRANRLE |
| | | SGVPSRFSGSGSGTDYTLTISSLQYEDFGIYYCQQYDESPWTF |
| | | GGGTKLEIKSGGGGSGALSNSIMYFSHFVPVFLPAKPTTTPAP |
| | | RPPTPAPTIASQPLSLRPEACRPAAGGAVHTRGLDIYIWAPLA |
| | | GTCGVLLLSLVITLYCRLKIQVRKAAITSYEKSDGVYTGLSTR |
| | | NQETYETLKHEKPPQGSGSMSNGYSTDENFRYLISCFRARVK |
| | | MYIQVEPVLDYLTFLPAEVKEQIQRTVATSGNMQAVELLLST |
| | | LEKGVWHLGWTREFVEALRRTGSPLAARYMNPELTDLPSPS |
| | | FENAHDEYLQLLNLLQPTLVDKLLVRDVLDKCMEEELLTIED |
| | | RNRIAAAENNGNESGVRELLKRIVQKENWFSAFLNVLRQTG |
| | | NNELVQELTGSDCSESNAEIEN |
| 28 | CD5-FcRγ-TNFR1 | MWLQSLLLLGTVACSISEIQLVQSGGGLVKPGGSVRISCAAS |
| | | GYTFTNYGMNWVRQAPGKGLEWMGWINTHTGEPTYADSF |
| | | KGRFTFSLDDSKNTAYLQINSLRAEDTAVYFCTRRGYDWYF |
| | | DVWGQGTTVTVSSGGGGSGGGGSGGGGSDIQMTQSPSSLSA |
| | | SVGDRVTITCRASQDINSYLSWFQQKPGKAPKTLIYRANRLE |
| | | SGVPSRFSGSGSGTDYTLTISSLQYEDFGIYYCQQYDESPWTF |
| | | GGGTKLEIKSGGGGSGALSNSIMYFSHFVPVFLPAKPTTTPAP |
| | | RPPTPAPTIASQPLSLRPEACRPAAGGAVHTRGLDIYIWAPLA |
| | | GTCGVLLLSLVITLYCRLKIQVRKAAITSYEKSDGVYTGLSTR |
| | | NQETYETLKHEKPPQGSGSQRWKSKLYSIVCGKSTPEKEGEL |
| | | EGTTTKPLAPNPSFSPTPGFTPTLGFSPVPSSTFTSSSTYTPGDC |
| | | PNFAAPRREVAPPYQGADPILATALASDPIPNPLQKWEDSAH |
| | | KPQSLDTDDPATLYAVVENVPPLRWKEFVRRLGLSDHEIDRL |
| | | ELQNGRCLREAQYSMLATWRRRTPRREATLELLGRVLRDM |
| | | DLLGCLEDIEEALCGPAALPPAPSLLR |
| 29 | CD5-FcRγ-TNFR2 | MWLQSLLLLGTVACSISEIQLVQSGGGLVKPGGSVRISCAAS |
| | | GYTFTNYGMNWVRQAPGKGLEWMGWINTHTGEPTYADSF |
| | | KGRFTFSLDDSKNTAYLQINSLRAEDTAVYFCTRRGYDWYF |
| | | DVWGQGTTVTVSSGGGGSGGGGSGGGGSDIQMTQSPSSLSA |
| | | SVGDRVTITCRASQDINSYLSWFQQKPGKAPKTLIYRANRLE |
| | | SGVPSRFSGSGSGTDYTLTISSLQYEDFGIYYCQQYDESPWTF |
| | | GGGTKLEIKSGGGGSGALSNSIMYFSHFVPVFLPAKPTTTPAP |
| | | RPPTPAPTIASQPLSLRPEACRPAAGGAVHTRGLDIYIWAPLA |
| | | GTCGVLLLSLVITLYCRLKIQVRKAAITSYEKSDGVYTGLSTR |
| | | NQETYETLKHEKPPQGSGSPLCLQREAKVPHLPADKARGTQ |
| | | GPEQQHLLITAPSSSSSSLESSASALDRRAPTRNQPQAPGVEA |
| | | SGAGEARASTGSSDSSPGGHGTQVNVTCIVNVCSSSDHSSQC |
| | | SSQASSTMGDTDSSPSESPKDEQVPFSKEECAFRSQLETPETL |
| | | LGSTEEKPLPLGVPDAGMKPS |

TABLE 1B
Linker sequences

| SEQ ID | Sequence |
|---|---|
| 30 | SSGGGGSGGGGSGGGGS |
| 31 | SGGGGSG |
| 32 | SGGG |
| 33 | GSGS |

In some embodiments, the therapeutically effective myeloid cell comprises or presents or expresses one or more an exogenous or recombinant tumor antigens. In some embodiments, the tumor antigens are tissue specific antigens. In some embodiments, the tumor antigens are endogenous overexpressed antigens. In some embodiments, the tumor antigens are mutated protein antigens. In some embodiments, the therapeutically effective myeloid cell comprises or presents or expresses a melanoma antigen such as a Tyrosinase-related Protein 2 (TRP2) antigen, such as a TRP2 epitope, such as amino acids 180-188 of TRP2 (SVYDFFVWL). In some embodiments, the therapeutically effective myeloid cell comprises or presents or expresses a mutant antigen, for example a glioblastoma antigen, e.g., an isocitrate dehydrogenase 1 (mIDHI) antigen, such as a mutant IDH1 antigen (R132H), such as GWVKPIIIGHHAYGDQYRATDFVVP. In some embodiments, a method can comprise administering a myeloid cell that comprises or presents or expresses one or more an exogenous or recombinant antigens to treat a cancer, such as a brain cancer, a glioma or a glioblastoma.

5′ UTR A1:
(SEQ ID NO: 46)
GGGAAAUAAGAGAGAAAAGAAGAGUAAGAAGAAAUAUAAGAGCCACC
5′ UTR B1:
(SEQ ID NO: 47)
ACUCCUCCCCAUCCUCUCCCUCUGUCCCUCUGUCCCUCUGACCCUGCACU
GUCCCAGCACC
5′ UTR C1:
(SEQ ID NO: 48)
ACAUUUGCUUCUGACACAACUGUGUUCACUAGCAACCUCAAACAGACACC
5′ UTR D1:
(SEQ ID NO: 49)
CTTGCCCGCCGATATCTCTGCCGGGTGACTAGCTGCTTCCTTTCTCTCTC
GCGCGCGGTGTGGTGGCAGCAGGCGCAGCCCAGCCTCGAA
5′ UTR E1:
(SEQ ID NO: 50)
CTTCCTTTTTGTCCGACATCTTGACGAGGCTGCGGTGTCTGCTGCTATTC
TCCGAGCTTCGCA
5′ UTR F1:
(SEQ ID NO: 51)
AGCAATCCTTTCTTTCAGCTGGAGTGCTCCTCAGGAGCCAGCCCCACCCT
TAGAAAAG
3′ UTR A2:
(SEQ ID NO: 52)
UGAUAAUAGGCUGGAGCCUCGGUGGCCAUGCUUCUUGCCCCUUGGGCCUC
CCCCCAGCCCCUCCUCCCCUUCCUGCACCCGUACCCCCGUGGUCUUUGAA
UAAAGUCUGA
3′ UTR B2:
(SEQ ID NO: 53)
CAGGACACAGCCUUGGAUCAGGACAGAGACUUGGGGGCCAUCCUGCCCCU
CCAACCCGACAUGUGUACCUCAGCUUUUUCCCUCACUUGCAUCAAUAAAG
CUUCUGUGUUUGGAACAG
3′ UTR C2:
(SEQ ID NO: 54)
GCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAU
UCUGCCUAAUAAAA
3′ UTR C2-2x:
(SEQ ID NO: 55)
GCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAU
UCUGCCUAAUAAAAGCUCGCUUUCUUGCUGUCCAAUUUCUAUUAAAGGUU
CCUUUGUUCCCUAAGUCCAACUACUAAACUGGGGGAUAUUAUGAAGGGCC
UUGAGCAUCUGGAUUCUGCCUAAUAAAA
3′ UTR D2:
(SEQ ID NO: 56)
CTGGAGAGAATCACAGATGTGGAATATTTGTCATAAATAAATAATGAAAA
CCT
3′ UTR E2:
(SEQ ID NO: 57)
ATAGGTCCAACCAGCTGTACATTTGGAAAAATAAAACTTTATTAAATCAA
A
3′ UTR F2:
(SEQ ID NO: 58)
CAAGCACGCAGCAATGCAGCTCAAAACGCTTAGCCTAGCCACACCCCCAC
GGGAAACAGCAGTGATTAACCTTTAGCAATAAACGAAAGTTTAACTAAGC
TATACTAACCCCAGGGTTGGTCAATTTCGTGCCAGCCACACCCTGGTACT
GCATGCACGCAATGCTAGCTGCCCCTTTCCCGTCCTGGGTACCCCGAGTC
TCCCCCGACCTCGGGTCCCAGGTATGCTCCCACCTCCACCTGCCCCACTC
ACCACCTCTGCTAGTTCCAGACACCTCC
3′ UTR G2:
(SEQ ID NO: 59)
GCUGAAGAAGUGGGAAUGGGAGCACUCUGUCUUCUUUGCUAGAGAAGUGG
AGAGAAAAUACAAAAGGUAAAGCAGUUGAGAUUCUCUACAACCUAAAAAA
UUCCUAGGUGCUAUUUUCUUAUCCUUUGUACUGUAGCUAAAUGUACCUGA
GACAUAUUAGUCUUUGAAAAAUAAAGUUAUGUAAGGUUUUUUUUAUCUUU
AAAUAGCUCUGUGGGUUUUAACAUUUUUAUAAAGAUAUACCAA

### Methods for Preparing CFPs and Engineered Myeloid Cells

The method for preparing CAR-Ps comprise the steps of (1) screening for PSR subunit framework; (2) screening for antigen binding specificity; (3) CAR-P engineered nucleic acid constructs; (4) engineering cells and validation.

Screening for PSR subunit framework: As described above, the design of the receptor comprises at least of one phagocytic receptor domain, which enables the enhanced signaling of phagocytosis. In essence a large body of plasma membrane proteins can be screened for novel phagocytic functions or enhancements domains. Methods for screening phagocytic receptor subunits are known to one of skill in the art. Additional information can be found in The Examples section. In general, functional genomics and reverse engineering is often employed to obtain a genetic sequence encoding a functionally relevant protein polypeptide or a portion thereof. In some embodiments, primers and probes are constructed for identification, and or isolation of a protein, a polypeptide or a fragment thereof or a nucleic acid fragment encoding the same. In some embodiments, the primer or probe may be tagged for experimental identification. In some embodiments, tagging of a protein or a peptide may be useful in intracellular or extracellular localization.

Potential antibodies are screened for selecting specific antigen binding domains of high affinity. Methods of screening for antibodies or antibody domains are known to one of skill in the art. Specific examples provide further information. Examples of antibodies and fragments thereof include, but are not limited to IgAs, IgDs, IgEs, IgGs, IgMs, Fab fragments, F(ab′)2 fragments, monovalent antibodies, scFv fragments, scRv-Fc fragments, IgNARs, hcIgGs, V_{HH} antibodies, nanobodies, and alphabodies.

Commercially available antibodies can be adapted to generate extracellular domains of a chimeric receptor. Examples of commercially available antibodies include, but are not limited to: anti-HGPRT, clone 13H11.1 (EMD Millipore), anti-ROR1 (ab135669) (Abcam), anti-MUC1 [EP1024Y] (ab45167) (Abcam), anti-MUC16 [X75] (ab1107) (Abcam), anti-EGFRvIII [L8A4] (Absolute antibody), anti-Mesothelin [EPR2685 (2)] (ab134109) (Abcam), HER2 [3B5] (ab16901) (Abcam), anti-CEA (LS-C84299-1000) (LifeSpan BioSciences), anti-BCMA (ab5972) (Abcam), anti-Glypican 3 [9C2] (ab129381) (Abcam), anti-FAP (ab53066) (Abcam), anti-EphA2 [RM-0051-8F21] (ab73254) (Abcam), anti-GD2 (LS-0546315) (LifeSpan BioSciences), anti-CD19 [2E2B6B10] (ab31947) (Abcam), anti-CD20 [EP459Y] (ab78237) (Abcam), anti-CD30 [EPR4102] (ab134080) (Abcam), anti-CD33 [SP266](ab199432) (Abcam), anti-CD123 (ab53698) (Abcam), anti-CD133 (BioLegend), anti-CD123 (1A3H4) ab181789 (Abcam), and anti-CD171 (L1.1) (Invitrogen antibodies). Techniques for creating antibody fragments, such as scFvs, from known antibodies are routine in the art.

The engineered nucleic acid can be generated following molecular biology techniques known to one of skill in the art. The methods include but are not limited to designing primers, generating PCR amplification products, restriction digestion, ligation, cloning, gel purification of cloned product, bacterial propagation of cloned DNA, isolation and purification of cloned plasmid or vector. General guidance can be found in: Molecular Cloning of PCR Products: by Michael Finney, Paul E. Nisson, Ayoub Rashtchian in Current Protocols in Molecular Biology, Volume 56, Issue 1 (First published: 1 Nov. 2001); Recombinational Cloning by Jaehong Park, Joshua LaBaer in Current Protocols in Molecular Biology Volume 74, Issue 1 (First published: 15 May 2006) and others. In some embodiments specific amplification techniques may be used, such as TAS technique (Transcription-based Amplification System), described by Kwoh et al. in 1989; the 3SR technique, which are hereby incorporated by reference. (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

In some embodiments the engineered nucleic acid sequence is optimized for expression in human.

DNA, mRNA and Circular RNA: In some embodiments, naked DNA or messenger RNA (mRNA) may be used to introduce the nucleic acid inside the cell. In some embodiments, DNA or mRNA encoding the PFP is introduced into the phagocytic cell by lipid nanoparticle (LNP) encapsulation. mRNA is single stranded and may be codon optimized. In some embodiments the mRNA may comprise one or more modified or unnatural bases such as 5′-Methylcytosine, or Pseudouridine. mRNA may be 50-10,000 bases long. In one aspect the transgene is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long. In some embodiments, the mRNA may be about 12,000 bases long. In some embodiments, the mRNA comprises a transgene sequence that encodes a fusion protein. LNP encapsulated DNA or RNA can be used for transfecting myeloid cells, such as macrophages, or can be administered directly to a subject.

In some embodiments, circular RNA (circRNAs) encoding the PFP is used. In circular RNAs (circRNAs) the 3′ and 5′ ends are covalently linked, constitute a class of RNA. CircRNA may be delivered inside a cell or a subject using LNPs.

Delivery of Nucleic Acids into a Cell:

Nucleic acids encoding the CFP or PFP as described herein may be introduced to a cell, e.g. a myeloid cell, via different delivery approaches. A engineered nucleic acid as described herein may be introduced to a cell in vitro, ex vivo or in vivo. In some embodiments, a nucleic acid is introduced into a myeloid cell in the form of a plasmid or a vector. In some embodiments, the vector is a viral vector. In some embodiments, the vector is an expression vector, for example, a vector comprising one or more promoters, and other regulatory components, including enhancer binding sequence, initiation and terminal codons, a 5′ UTR, a 3′ UTR comprising a transcript stabilization element, optional conserved regulatory protein binding sequences and others. In some embodiments, the vector is a phage, a cosmid, or an artificial chromosome.

In some embodiments, a vector is introduced or incorporated in the cell by known methods of transfection, such as using lipofectamine, or calcium phosphate, or via physical means such as electroporation or nucleofection. In some embodiments the vector is introduced or incorporated in the cell by infection, a process commonly known as viral transduction.

In some embodiments, the vector for expression of the CFP is of a viral origin. In some embodiments, the engineered nucleic acid is encoded by a viral vector capable of replicating in non-dividing cells. In some embodiments, the nucleic acid encoding the engineered nucleic acid is encoded by a lentiviral vector, e.g. HIV and FIV-based vectors. In some embodiments the lentiviral vector is prepared in-house and manufactured in large scale for the purpose. In some embodiments, commercially available lentiviral vectors are utilized, as is known to one of skill in the art. In some embodiments, the engineered nucleic acid is encoded by a herpes simplex virus vector, a vaccinia virus vector, an adenovirus vector, or an adeno-associated virus (AAV) vector.

In some embodiments, a stable integration of transgenes into myeloid cells, such as macrophages, and other phagocytic cells may be accomplished via the use of a transposase and transposable elements, in particular, mRNA-encoded transposase. In one embodiment, Long Interspersed Element-1 (L1) RNAs may be contemplated for retrotransposition of the transgene and stable integration into myeloid cells, such as macrophages or phagocytic cells. Retrotransposon may be used for stable integration of a engineered nucleic acid encoding a phagocytic or tethering receptor (PR) fusion protein (PFP).

In some embodiments, the myeloid cell may be modified by expressing a transgene via incorporation of the transgene in a transient expression vector. In some embodiments expression of the transgene may be temporally regulated by a regulator from outside the cell. Examples include the Tet-on Tet-off system, where the expression of the transgene is regulated via presence or absence of tetracycline.

In some embodiments, the engineered nucleic acid described herein is a circular RNA (circRNA). A circular RNA comprises a RNA molecule where the 5′ end and the 3′ end of the RNA molecule are joined together. Without wishing to be bound by any theory, circRNAs have no free ends and may have longer half-life as compared to some other forms of RNAs or nucleic acid and may be resistant to digestion with RNase R exonuclease and turn over more slowly than its counterpart linear RNA in vivo. In some embodiments, the half-life of a circRNA is more than 20 hours. In some embodiments, the half-life of a circRNA is more than 30 hours. In some embodiments, the half-life of a circRNA is more than 40 hours. In some embodiments, the half-life of a circRNA is more than 48 hours. In certain embodiments, a circRNA comprises an internal ribosome entry site (IRES) element that engages a eukaryotic ribosome and an RNA sequence element encoding a polypeptide operatively linked to the IRES for insertion into cells in order to produce a polypeptide of interest.

circRNAs can be prepared by methods known to those skilled in the art. For example, circRNAs may be chemically synthesized and/or enzymatically synthesized, for example by enzymatically synthesis of the RNA followed by chemical joining of the ends of the RNA to form the circRNA. In some embodiments, a linear primary construct or linear mRNA may be cyclized, or concatemerized to create a circRNA. The mechanism of cyclization or concatemerization may occur through methods such as, but not limited to, chemical, enzymatic, or ribozyme catalyzed methods. The newly formed 5′-/3′-linkage may be an intramolecular linkage or an intermolecular linkage. In some embodiments, a linear primary construct or linear mRNA may be cyclized, or concatemerized using the chemical method to form a circRNA. In the chemical method, the 5′-end and the 3′-end of the nucleic acid (e.g., linear primary construct or linear mRNA) contain chemically reactive groups that, when close together, form a new covalent linkage between the 5′-end and the 3′-end of the molecule. The 5′-end may contain a NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond. In some embodiments, a DNA or RNA ligase, e.g. a T4 ligase, may be used to enzymatically link a 5′-phosphorylated nucleic acid molecule (e.g., a linear primary construct or linear mRNA) to the 3′-hydroxyl group of a nucleic acid forming a new phosphorodiester linkage. In some embodiments, a linear primary construct or linear mRNA may be cyclized or concatermerized by using at least one non-nucleic acid moiety. For example, the at least one non-nucleic acid moiety may react with regions or features near the 5′ terminus and/or near the 3′ terminus of the linear primary construct or linear mRNA in order to cyclize or concatermerize the linear primary construct or linear mRNA. In some embodiments, a linear primary construct or linear mRNA may be cyclized or concatermerized due to a non-nucleic acid moiety that causes an attraction between atoms, molecules surfaces at, near or linked to the 5′ and 3′ ends of the linear primary construct or linear mRNA. For example, a linear primary construct or linear mRNA may be cyclized or concatermized by intermolecular forces or intramolecular forces. Non-limiting examples of intermolecular forces. In some embodiments, a linear primary construct or linear mRNA may comprise a ribozyme RNA sequence near the 5′ terminus and near the 3′ terminus. In some embodiments, a circRNA may be synthesized by inserting DNA fragments into a plasmid containing sequences having the capability of spontaneous cleavage and self-circularization. In some embodiments, a circRNA is produced by making a DNA construct encoding an RNA cyclase ribozyme, expressing the DNA construct as an RNA, and then allowing the RNA to self-splice, which produces a circRNA free from intron in vitro. In some embodiments, a circRNA is produced by synthesizing a linear polynucleotide, combining the linear nucleotide with a complementary linking oligonucleotide under hybridization conditions, and ligating the linear polynucleotide.

The circRNA may be modified or unmodified. In some embodiments, the circRNA is chemically modified. For example, an A, G, U or C ribonucleotide of a circRNA may comprise chemical modifications. In some embodiments, any region of a circRNA, e.g. the coding region of the CFP or PFP, the flanking regions and/or the terminal regions may contain one, two, or more (optionally different) nucleoside or nucleotide modifications. In some embodiments, a modified circRNA introduced to a cell may exhibit reduced degradation in the cell, as compared to an unmodified circRNA. Modifications such as to the sugar, the nucleobase, or the internucleoside linkage (e.g. to a linking phosphate/to a phosphodiester linkage/to the phosphodiester backbone) are also encompassed. In some embodiments, one or more atoms of nucleobase, e.g. a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Additional modifications to circRNAs are described in US20170204422, the entire content of which is incorporated herein by reference.

In some embodiments, the circRNA is conjugated to other polynucleotides, dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, or cofactors.

In some embodiments, the circRNA is administered directly to tissues of a subject. Additional description of circRNAs in U.S. Pat. Nos. 5,766,903, 5,580,859, 5,773,244, 6,210,931, PCT publication No. WO1992001813, Hsu et al., Nature (1979) 280:339-340, Harland & Misher, Development (1988) 102:837-852, Memczak et al. Nature (2013) 495:333-338, Jeck et al., and RNA (2013) 19:141-157, each of which is incorporated herein by reference in its entirety.

In some embodiments, a nucleic acid is introduced into a myeloid cell with a nanoparticle (NP). A nanoparticle may be of various shapes or sizes and may harbor the nucleic acid encoding the CFP or PFP. In some embodiments, the NP is a lipid nanoparticle (LNP). In some embodiments, the NP comprises poly(amino acids), polysaccharides and poly(alpha-hydroxy acids), gold, silver, carbon, iron, silica, or any combination thereof. In some embodiments, the NP comprises a polylactide-co-glycolide (PGLA) particle. In some embodiments, the nucleic acid is encapsulated in the NP, for example, via water/oil emulsion or water-oil-water emulsion. In some embodiments, the nucleic acid is conjugated to the NP.

Nanoparticles (NPs) may be delivered to a cell in vitro, ex vivo or in vivo. In some embodiments, a NP is delivered to a phagocytic cell ex vivo. In some embodiments, a NP is delivered to a phagocytic cell in vivo. In some embodiments, the NP is less than 100 nm in diameter. In some embodiments, the NP is more than 100 nm in diameter. In some embodiments, the NP is a rod-shaped NP. In some embodiments, the NP is a spherical NP. In particular embodiments, the NP is a spherical NP for delivery to a phagocytic cell. In additional embodiments, the NP is at least 100 nm in diameter and does not trigger or triggers reduced toxicity when delivered to a cell.

In some embodiments, the NP is positively charged. In some embodiments, the NP is negatively charged. In some embodiments, the NP is neutral. In some embodiments, the NP is a cationic NP that is delivered and taken up by a myeloid cell ex vivo or in vivo.

Stiffness may affect the biological impact of NPs. NPs made of rigid materials may be associated with increased potential for embolism, while flexible polymer-based NPs that can more easily deform may gain better access to tissues during the complex vascular changes associated with inflammation. The fluidity of NPs, too, affects the ability of antigen-loaded NP to stimulate immune responses. Thus, intramuscular, solid-phase, antigen-containing liposome immunization may elicit a more robust Th1/Th17 response than similarly administered fluid-phase liposomes. Without wishing to be bound by any theory, solid-phase particles may result from the formation of an immobilized antigen particle depot and may result in a prolonged supply of antigen for APCs also associated with upregulation of positive costimulatory molecules such as CD80, which support efficient T cell priming.

In some embodiments, a protein corona may form around NPs. A protein corona may form in a two-step process. In the first step, high-affinity proteins rapidly bind to NPs to form a primary corona. In the second step, proteins of lower affinity bind either directly to the NP or to the proteins in the primary corona forming a secondary corona. Constituents of the protein corona may thus be impacted by the protein content of the serum and thus by the homeostatic or immune responses that regulate it. In some embodiments, proteins with high abundance, such as albumin, comprise a significant proportion of the primary corona. In some embodiments, NPs with different charges bind significant amounts of less-abundant proteins in particular environments, e.g. in plasma with certain antigen or antibody. In vivo formation of a protein corona may alter NP charge or mask functional groups important for NP targeting to certain receptors and/or enhance clearance of NPs by phagocytes. In some embodiments, NPs are engineered to reduce changes to NP charges or masking of functional groups, and/or increase the serum half-life of the NPs. In some embodiments, the NP comprises lipid anchored PEG moieties. In some embodiments, NP surface coatings are designed to modulate opsonization events. For example, the NP's surface may be coated with polymeric ethylene glycol (PEG) or its low molecular weight derivative polyethylene oxide (PEO). Without wishing to be bound by any theory, PEG increases surface hydrophilicity and can prevent NPs from merging with aqueous solutions. In some embodiments, the NP coated with PEG or PEO are engineered to result in reduced toxicity or increased biocompatibility of the NPs. Additional NP design and NP targeting for myeloid cells are described in Getts et al., Trends Immunol. 36(7): 419-427 (2015), the entirety of which is incorporated herein by reference. In some embodiments, the NP comprises an ionizable lipid, a sterol. In some embodiments, the NP comprises a PEG lipid, a phospholipid and cholesterol.

NPs described herein may be used to introduce the engineered nucleic acid into a cell in in vitro/ex vivo cell culture or administered in vivo. In some embodiments, the NP is modified for in vivo administration. For example, the NP may comprise surface modification or attachment of binding moieties to bind specific toxins, proteins, ligands, or any combination thereof, before being taken up by liver or spleen phagocytes. In recent rodent proof-of-concept studies, infused highly negatively charged ‘immune-modifying NPs’ (IMPs) can absorb certain blood proteins, including S100 family and heat shock proteins, before finally being removed and destroyed by cells of the mononuclear phagocyte system. Furthermore, this mechanism may also be used to capture and concentrate certain circulating proteins. IMPs have been shown to bind Annexin 1. The accumulation of Annexin 1 and its presentation to particular leukocyte subsets can have broad immune outcomes. For example, Annexin 1-loaded NPs may reduce neutrophils via induction of apoptosis and/or promote T cell activation. In some embodiments, the NP is designed to target a cell surface receptor, e.g. a scavenger receptor. In some embodiments, a NP is a particle with a negative surface charge.

In some embodiments, the NP encapsulates the nucleic acid wherein the nucleic acid is a naked DNA molecule. In some embodiments, the NP encapsulates the nucleic acid wherein the nucleic acid is an mRNA molecule. In some embodiments, the NP encapsulates the nucleic acid wherein the nucleic acid is a circular RNA (circRNA) molecule. In some embodiments, the NP encapsulates the nucleic acid wherein the nucleic acid is a vector, a plasmid, or a portion or fragment thereof.

In some embodiments, the NP is a Lipid nanoparticle (LNP). LNPs may comprise a polar and or a nonpolar lipid. In some embodiments cholesterol is present in the LNPs for efficient delivery. LNPs are 100-300 nm in diameter provide efficient means of mRNA delivery to various cell types, including myeloid cells, such as macrophages. In some embodiments, LNP may be used to introduce the nucleic acids into a cell in in vitro cell culture. In some embodiments, the LNP encapsulates the nucleic acid wherein the nucleic acid is a naked DNA molecule. In some embodiments, the LNP encapsulates the nucleic acid wherein the nucleic acid is an mRNA molecule. In some embodiment described herein, lipid nanoparticles are formed associating or encapsulating the full length recombinant (engineered) mRNA. In some embodiments, the number of mRNA molecules per LNP is regulated for optimum delivery of the mRNA inside the cell. In some embodiments, the LNP is used to deliver mRNA systemically, that may be taken up by myeloid cells in vivo. In some embodiments, the LNP may comprise target moieties.

In some embodiments, the LNP does not comprise myeloid cell-targeting moieties on the surface, but the mRNA is designed for myeloid cell-specific expression. In some embodiments, the LNP encapsulates the nucleic acid wherein the nucleic acid is inserted in a vector, such as a plasmid vector. In some embodiments, the LNP encapsulates the nucleic acid wherein the nucleic acid is a circRNA molecule.

In some embodiments, mRNA can be encapsidated within mammalian retro-viral like PEG10 packages that deliver the mRNA inside a cell. Specific fusogens may be used for cell targeting with PEG10 delivery to organ, tissue or cells, e.g., myeloid cells. PEG10 is known to bind to its own mRNA and deliver it inside a cell. PEG10 UTR regions may be incorporated flanking the coding region of the mRNA, to facilitate PEG10 encapsidation. (Segel et al., Science (2021), 373: 6557, p882-889).

In some embodiments, the LNP is used to deliver the nucleic acid into the subject. LNP can be used to deliver nucleic acid systemically in a subject. It can be delivered by injection. In some embodiments, the LNP comprising the nucleic acid is injected by intravenous route. In some embodiments the LNP is injected subcutaneously. In some embodiments the LNP is injected intramuscularly.

### Pharmaceutical Composition

Provided herein is a pharmaceutical composition, comprising engineered myeloid cells, such as macrophages, comprising a engineered nucleic acid encoding the CFP and a pharmaceutically acceptable excipient.

Also provided herein is a pharmaceutical composition, comprising a nucleic acid encoding the CFP and a pharmaceutically acceptable excipient. The pharmaceutical composition may comprise DNA, mRNA or circRNA or an LNP or a liposomal composition comprising any one of these.

Also provided herein is a pharmaceutical composition comprising a vector comprising the engineered nucleic acid encoding the CFP and a pharmaceutically acceptable excipient. The pharmaceutical composition may comprise DNA, mRNA or circRNA inserted in a plasmid vector or a viral vector.

In some embodiments the engineered myeloid cells, such as macrophages, are grown in cell culture sufficient for a therapeutic administration dose, and washed, and resuspended into a pharmaceutical composition.

In some embodiments the excipient comprises a sterile buffer, (e.g. HEPES or PBS) at neutral pH. In some embodiment, the pH of the pharmaceutical composition is at 7.5. In some embodiments, the pH may vary within an acceptable range. In some embodiments, the engineered cells may be comprised in sterile enriched cell suspension medium comprising complement deactivated or synthetic serum. In some embodiments the pharmaceutic composition further comprises cytokines, chemokines or growth factors for cell preservation and function.

In some embodiments, the pharmaceutical composition may comprise additional therapeutic agents, co-administered with the engineered cells.

### Treatment Methods

Provided herein are methods for treating cancer in a subject using a pharmaceutical composition comprising engineered myeloid cells, such as phagocytic cells (e.g., macrophages), expressing a engineered nucleic acid encoding a CFP, such as a phagocytic receptor (PR) fusion protein (PFP), to target, attack and kill cancer cells directly or indirectly. The engineered myeloid cells, such as phagocytic cells, are also designated as CAR-P cells in the descriptions herein.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some aspects, any gene of interest can be expressed in a myeloid cell, such that the cell can be used to treat a disease that requires, for example an active phagocytic cell, such as an infection, where the myeloid cell may be specifically engineered to target, engulf and destroy the pathogen.

In general, cellular immunotherapy comprises providing the patient a medicament comprising live cells. In some aspects a patient or a subject having cancer, is treated with autologous cells, the method comprising, isolation of PBMC-derived myeloid cells, such as macrophages, modifying the cells ex vivo to generate phagocytic myeloid cells capable of tumor lysis by introducing into the cells a engineered nucleic acid encoding a CFP, and administering the modified myeloid cells into the subject.

In some aspects, a subject is administered one or more doses of a pharmaceutical composition comprising therapeutic myeloid cells, such as phagocytic cells, wherein the cells are allogeneic. An HLA may be matched for compatibility with the subject, and such that the cells do not lead to graft versus Host Disease, GVHD. A subject arriving at the clinic is HLA typed for determining the HLA antigens expressed by the subject.

HLA-typing is conventionally carried out by either serological methods using antibodies or by PCR-based methods such as Sequence Specific Oligonucleotide Probe Hybridization (SSOP), or Sequence Based Typing (SBT).

The sequence information may be identified by either sequencing methods or methods employing mass spectrometry, such as liquid chromatography—mass spectrometry (LC-MS or LC-MS/MS, or alternatively HPLC-MS or HPLC-MS/MS). These sequencing methods may be well-known to a skilled person and are reviewed in Medzihradszky K F and Chalkley R J. Mass Spectrom Rev. 2015 January-February; 34(1):43-63.

In some aspects, the phagocytic cell is derived from the subject, electroporated, transfected or transduced with the engineered nucleic acid in vitro, expanded in cell culture in vitro for achieving a number suitable for administration, and then administered to the subject. In some embodiments, the steps of electroporated, transfected or transduced with the engineered nucleic acid in vitro, expanded in cell culture in vitro for achieving a number suitable for administration takes 2 days, or 3 days, or 4 days or 5 days or 6 days or 7 days or 8 days or 9 days or 10 days.

In some embodiments, sufficient quantities of electroporated, transfected or transduced myeloid cells, such as macrophages, comprising the engineered nucleic acid are preserved aseptically, which are administered to the subject as “off the shelf” products after HLA typing and matching the product with the recipients HLA subtypes. In some embodiments, the engineered phagocytes are cryopreserved. In some embodiments, the engineered phagocytes are cryopreserved in suitable media to withstand thawing without considerable loss in cell viability.

In some embodiment, the subject is administered a pharmaceutical composition comprising the DNA, or the mRNA or the circRNA in a vector, or in a pharmaceutically acceptable excipient described above.

In some embodiments the administration of the off the shelf cellular products may be instantaneous, or may require 1 day, 2 days or 3 days or 4 days or 5 days or 6 days or 7 days or more prior to administration. The pharmaceutical composition comprising cell, or nucleic acid may be preserved over time from preparation until use in frozen condition. In some embodiments, the pharmaceutical composition may be thawed once. In some embodiments, the pharmaceutical composition may be thawed more than once. In some embodiments, the pharmaceutical composition is stabilized after a freeze-thaw cycle prior administering to the subject. In some embodiments the pharmaceutical composition is tested for final quality control after thawing prior administration.

In some embodiments, a composition comprising 10\hat{ }6 engineered cells are administered per administration dose. In some embodiments, a composition comprising 10\hat{ }7 engineered cells are administered per administration dose. In some embodiments, a composition comprising 5×10\hat{ }7 engineered cells are administered per administration dose. In some embodiments, a composition comprising 10\hat{ }8 engineered cells are administered per administration dose. In some embodiments, a composition comprising 2×10\hat{ }8 engineered cells are administered per administration dose. In some embodiments, a composition comprising 5×10\hat{ }8 engineered cells are administered per administration dose. In some embodiments, a composition comprising 10\hat{ }9 engineered cells are administered per administration dose. In some embodiments, a composition comprising 10′10 engineered cells are administered per administration dose.

In some embodiments, the engineered myeloid cells, such as phagocytic cells, are administered once.

In some embodiments, the engineered myeloid cells, such as phagocytic cells, are administered more than once.

In some embodiments, the engineered myeloid cells, such as phagocytic cells, are twice, thrice, four times, five times, six times, seven times, eight times, nine times, or ten times or more to a subject over a span of time comprising a few months, a year or more.

In some embodiments, the engineered myeloid cells, such as phagocytic cells, are administered twice weekly.

In some embodiments, the engineered myeloid cells, such as phagocytic cells, are administered once weekly.

In some embodiments, the engineered myeloid cells, such as phagocytic cells, are administered once every two weeks.

In some embodiments, the engineered myeloid cells, such as phagocytic cells, are administered once every three weeks.

In some embodiments, the engineered myeloid cells, such as phagocytic cells, are administered once monthly.

In some embodiments, the engineered phagocytic cells are administered once in every 2 months, once in every 3 months, once in every 4 months, once in every 5 months or once in every 6 months.

In some embodiments, the engineered myeloid cells, such as phagocytic cells, are administered by injection.

In some embodiments, the engineered myeloid cells, such as phagocytic cells, are administered by infusion.

In some embodiments, the engineered myeloid cells, such as phagocytic cells, are administered by intravenous infusion.

In some embodiments, the engineered myeloid cells, such as phagocytic cells, are administered by subcutaneous infusion.

The pharmaceutical composition comprising the engineered nucleic acid or the engineered cells may be administered by any route which results in a therapeutically effective outcome. These include, but are not limited to enteral (into the intestine), gastroenteral, epidural (into the dura mater), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier.

In some embodiments, the subject is administered a pharmaceutical composition comprising the nucleic acid encoding the CFP or PFP as described herein. In some embodiments, the subject is administered a pharmaceutical composition comprising DNA, mRNA, or circRNA. In some embodiments, the subject is administered a vector harboring the nucleic acid, e.g., DNA, mRNA, or circRNA. In some embodiments, the nucleic acid is administered or in a pharmaceutically acceptable excipient described above.

In some embodiments, the subject is administered a nanoparticle (NP) associated with the nucleic acid, e.g. a DNA, an mRNA, or a circRNA encoding the CFP or PFP as described herein. In some embodiments, the nucleic acid is encapsulated in the nanoparticle. In some embodiments, the nucleic acid is conjugated to the nanoparticle. In some embodiments, the NP is a polylactide-co-glycolide (PGLA) particle. In some embodiments, the NP is administered subcutaneously. In some embodiments, the NP is administered intravenously. In some embodiments, the NP is engineered in relation to the administration route. For example, the size, shape, or charges of the NP may be engineered according to the administration route. In some embodiments, subcutaneously administered NPs are less than 200 nm in size. In some embodiments, subcutaneously administered NPs are more than 200 nm in size. In some embodiments, subcutaneously administered NPs are at least 30 nm in size. In some embodiments, the NPs are intravenously infused. In some embodiments, intravenously infused NPs are at least 5 nm in diameter. In some embodiments, intravenously infused NPs are at least 30 nm in diameter. In some embodiments, intravenously infused NPs are at least 100 nm in diameter. In certain embodiments, the administered NPs, e.g. intravenously administered NPs, are engulfed by circulating monocytes. Additional NP design and administration approaches are described in Getts et al., Trends Immunol. 36(7): 419-427 (2015), the entirety of which is incorporated herein by reference.

In some embodiments, the subject is administered a pharmaceutical composition comprising a circRNA encoding the CFP or PFP as described herein. The circRNA may be administered in any route as described herein. In some embodiments, the circRNA may be directly infused. In some embodiments, the circRNA may be in a formulation or solution comprising one or more of sodium chloride, calcium chloride, phosphate and/or EDTA. In some embodiment, the circRNA solution may include one or more of saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In some embodiments, the circRNA solution is lyophilized. The amount of each component may be varied to enable consistent, reproducible higher concentration saline or simple buffer formulations. The components may also be varied in order to increase the stability of circRNA in the buffer solution over a period of time and/or under a variety of conditions. In some embodiments, the circRNA is formulated in a lyophilized gel-phase liposomal composition. In some embodiments, the circRNA formulation comprises a bulking agent, e.g. sucrose, trehalose, mannitol, glycine, lactose and/or raffinose, to impart a desired consistency to the formulation and/or stabilization of formulation components. Additional formulation and administration approaches for circRNA as described in US Publications No. US2012060293, and US20170204422 are herein incorporated by reference in entirety.

In some embodiments, the subject is administered a pharmaceutical composition comprising a mRNA encoding the CFP or PFP as described herein. In some embodiments, the mRNA is co-formulated into nanoparticles (NPs), such as lipid nanoparticles (LNPs). For example, the LNP may comprise cationic lipids or ionizable lipids. In some embodiments, the mRNA is formulated into polymeric particles, for example, polyethyleneimine particles, poly(glycoamidoamine), ly(β-amino)esters (PBAEs), PEG particles, ceramide-PEGs, polyamindoamine particles, or polylactic-co-glycolic acid particles (PLGA). In some embodiments, the mRNA is administered by direct injection. In some embodiments, the mRNA is complexed with transfection agents, e.g. Lipofectamine 2000, jetPEI, RNAiMAX, or Invivofectamine.

The mRNA may be a naked mRNA. The mRNA may be modified or unmodified. For example, the mRNA may be chemically modified. In some embodiments, nucleobases and/or sequences of the mRNA are modified to increase stability and half-life of the mRNA. In some embodiments, the mRNA is glycosylated. Additional mRNA modification and delivery approaches as described in Flynn et al., BioRxiv 787614 (2019) and Kowalski et al. Mol. Ther. 27(4): 710-728 (2019) are each incorporated herein by reference in its entirety.

### EXAMPLES

### Example 1. Experimental Methods for Testing Myeloid Cell Activation and Function

In this section, an exemplary design for.

### Inflammasome Activation Assay:

Activation of NLRP3 inflammasome is assayed by ELISA detection of increased IL-1 production and detection caspase-1 activation by western blot, detecting cleavage of procaspase to generate the shorter caspase. In a microwell plate multiplex setting, Caspase-Glo (Promega Corporation) is used for faster readout of Caspase 1 activation.

iNOS Activation Assay:

Activation of the oxidative burst potential is measured by iNOS activation and NO production using a fluorimetric assay NOS activity assay kit (AbCAM).

### Cancer Cell Killing Assay:

Raji B cells are used as cancer antigen presenting cells. Raji cells are incubated with whole cell crude extract of cancer cells, and co-incubated with J774 macrophage cell lines. The macrophages can destroy the cells after 1 hour of infection, which can be detected by microscopy or detected by cell death assay.

### Screening for High Affinity Antigen Binding Domains:

Cancer ligands are subjected to screening for antibody light chain and heavy chain variable domains to generate extracellular binding domains for the CFPs. Human full length antibodies or scFv libraries are screened. Also potential ligands are used for immunizing llama for development of novel immunoglobulin binding domains in llama, and preparation of single domain antibodies.

Specific useful domains identified from the screens are then reverse transcribed, and cloned into lentiviral expression vectors to generate the CFP constructs. An engineered nucleic acid encoding a CFP can generated using one or more domains from the extracellular, TM and cytoplasmic regions of the highly phagocytic receptors generated from the screen. Briefly plasma membrane receptors showing high activators of pro-inflammatory cytokine production and inflammasome activation are identified. Bioinformatics studies are performed to identify functional domains including extracellular activation domains, transmembrane domains and intracellular signaling domains, for example, specific kinase activation sites, SH2 recruitment sites. These screened functional domains are then cloned in modular constructions for generating novel CFPs. These are candidate CFPs, and each of these chimeric construct is tested for phagocytic enhancement, production of cytokines and chemokines, and/or tumor cell killing in vitro and/or in vivo. A microparticle based phagocytosis assay was used to examine changes in phagocytosis. Briefly, streptavidin coupled fluorescent polystyrene microparticles (6 μm diameter) are conjugated with biotinylated recombinantly expressed and purified cancer ligand. Myeloid cells expressing the novel CFP were incubated with the ligand coated microparticles for 1-4 h and the amount of phagocytosis was analyzed and quantified using flow cytometry. Plasmid or lentiviral constructions of the designer CFPs are then prepared and tested in macrophage cells for cancer cell lysis.

Exemplary functional domain containing CFPs are described in the following sections.

### Example 2. Myeloid/Macrophage Cell Isolation from PBMCs

Peripheral blood mononuclear cells are separated from normal donor buffy coats by density centrifugation using Histopaque 1077 (Sigma). After washing, CD14+ monocytes are isolated from the mononuclear cell fraction using CliniMACS GMP grade CD14 microbeads and LS separation magnetic columns (Miltenyi Biotec). Briefly, cells are resuspended to appropriate concentration in PEA buffer (phosphate-buffered saline [PBS] plus 2.5 mmol/L ethylenediaminetetraacetic acid [EDTA] and human serum albumin [0.5% final volume of Alburex 20%, Octopharma]), incubated with CliniMACS CD14 beads per manufacturer's instructions, then washed and passed through a magnetized LS column. After washing, the purified monocytes are eluted from the demagnetized column, washed and re-suspended in relevant medium for culture. Isolation of CD14+ cells from leukapheresis: PBMCs are collected by leukapheresis from cirrhotic donors who gave informed consent to participate in the study. Leukapheresis of peripheral blood for mononuclear cells (MNCs) is carried out using an Optia apheresis system by sterile collection. A standard collection program for MNC is used, processing 2.5 blood volumes. Isolation of CD14 cells is carried out using a GMP-compliant functionally closed system (CliniMACS Prodigy system, Miltenyi Biotec). Briefly, the leukapheresis product is sampled for cell count and an aliquot taken for pre-separation flow cytometry. The percentage of monocytes (CD14+) and absolute cell number are determined, and, if required, the volume is adjusted to meet the required criteria for selection (≤20×10⁹ total white blood cells; <400×10⁶ white blood cells/mL; ≤3.5×10⁹ CD14 cells, volume 50-300 mL). CD14 cell isolation and separation is carried out using the CliniMACS Prodigy with CliniMACS CD14 microbeads (medical device class III), TS510 tubing set and LP-14 program. At the end of the process, the selected CD14+ positive monocytes are washed in PBS/EDTA buffer (CliniMACS buffer, Miltenyi) containing pharmaceutical grade 0.5% human albumin (Alburex), then re-suspended in TexMACS (or comparator) medium for culture.

### Cell Count and Purity:

Cell counts of total MNCs and isolated monocyte fractions are performed using a Sysmex XP-300 automated analyzer (Sysmex). Assessment of macrophage numbers is carried out by flow cytometry with TruCount tubes (Becton Dickinson) to determine absolute cell number, as the Sysmex consistently underestimated the number of monocytes. The purity of the separation is assessed using flow cytometry (FACSCanto II, BD Biosciences) with a panel of antibodies against human leukocytes (CD45-VioBlue, CD15-FITC, CD14-PE, CD16-APC), and product quality is assessed by determining the amount of neutrophil contamination (CD45int, CD15pos).

Cell Culture—Development of Cultures with Healthy Donor Samples

Optimal culture medium for macrophage differentiation is investigated, and three candidates are tested using for the cell product. In addition, the effect of monocyte cryopreservation on deriving myeloid cells and macrophages for therapeutic use is examined. Functional assays are conducted to quantify the phagocytic capacity of myeloid cells and macrophages and their capacity for further polarization, and phagocytic potential as described elsewhere in the disclosure.

Full-Scale Process Validation with Subject Samples

Monocytes cultured from leukapheresis from Prodigy isolation are cultured at 2×10⁶ monocytes per cm² and per mL in culture bags (MACS GMP differentiation bags, Miltenyi) with GMP-grade TexMACS (Miltenyi) and 100 ng/mL M-CSF. Monocytes are cultured with 100 ng/mL GMP-compliant recombinant human M-CSF (R&D Systems). Cells are cultured in a humidified atmosphere at 37° C., with 5% CO₂ for 7 days. A 50% volume media replenishment is carried out twice during culture (days 2 and 4) with 50% of the culture medium removed, then fed with fresh medium supplemented with 200 ng/mL M-CSF (to restore a final concentration of 100 ng/mL).

### Cell Harvesting:

For normal donor-derived macrophages, cells are removed from the wells at day 7 using Cell Dissociation Buffer (Gibco, Thermo Fisher) and a pastette. Cells are resuspended in PEA buffer and counted, then approximately 1×10⁶ cells per test are stained for flow cytometry. Leukapheresis-derived macrophages are removed from the culture bags at day 7 using PBS/EDTA buffer (CliniMACS buffer, Miltenyi) containing pharmaceutical grade 0.5% human albumin from serum (HAS; Alburex). Harvested cells are resuspended in excipient composed of two licensed products: 0.9% saline for infusion (Baxter) with 0.5% human albumin (Alburex).

### Flow Cytometry Characterization:

Monocyte and macrophage cell surface marker expression is analyzed using either a FACSCanto II (BD Biosciences) or MACSQuant 10 (Miltenyi) flow cytometer. Approximately 20,000 events are acquired for each sample. Cell surface expression of leukocyte markers in freshly isolated and day 7 matured cells is carried out by incubating cells with specific antibodies (final dilution 1:100). Cells are incubated for 5 min with FcR block (Miltenyi) then incubated at 4° C. for 20 min with antibody cocktails. Cells are washed in PEA, and dead cell exclusion dye DRAQ7 (BioLegend) is added at 1:100. Cells are stained for a range of surface markers as follows: CD45-VioBlue, CD14-PE or CD14-PerCP-Vio700, CD163-FITC, CD169-PE and CD16-APC (all Miltenyi), CCR2-BV421, CD206-FITC, CXCR4-PE and CD115-APC (all BioLegend), and 25F9-APC and CD115-APC (eBioscience). Both monocytes and macrophages are gated to exclude debris, doublets and dead cells using forward and side scatter and DRAQ7 dead cell discriminator (BioLegend) and analyzed using FlowJo software (Tree Star). From the initial detailed phenotyping, a panel is developed as Release Criteria (CD45-VB/CD206-FITC/CD14-PE/25F9 APC/DRAQ7) that defined the development of a functional macrophage from monocytes. Macrophages are determined as having mean fluorescence intensity (MFI) five times higher than the level on day 0 monocytes for both 25F9 and CD206. A second panel is developed which assessed other markers as part of an Extended Panel, composed of CCR2-BV421/CD163-FITC/CD169-PE/CD14-PerCP-Vio700/CD16-APC/DRAQ7), but is not used as part of the Release Criteria for the cell product.

Both monocytes and macrophages from buffy coat CD14 cells are tested for phagocytic uptake using pHRodo beads, which fluoresce only when taken into acidic endosomes. Briefly, monocytes or macrophages are cultured with 1-2 uL of pHRodo Escherichia coli bioparticles (LifeTechnologies, Thermo Fisher) for 1 h, then the medium is taken off and cells washed to remove non-phagocytosed particles. Phagocytosis is assessed using an EVOS microscope (Thermo Fisher), images captured and cellular uptake of beads quantified using ImageJ software (NIH freeware). The capacity to polarize toward defined differentiated macrophages is examined by treating day 7 macrophages with IFNγ (50 ng/mL) or IL-4 (20 ng/mL) for 48 h to induce polarization to M1 or M2 phenotype (or M[IFNγ] versus M[IL-4], respectively). After 48 h, the cells are visualized by EVOS bright-field microscopy, then harvested and phenotyped as before. Further analysis is performed on the cytokine and growth factor secretion profile of macrophages after generation and in response to inflammatory stimuli. Macrophages are generated from healthy donor buffy coats as before, and either left untreated or stimulated with TNFα (50 ng/mL, Peprotech) and polyinosinic:polycytidylic acid (poly I:C, a viral homolog which binds TLR3, 1 g/mL, Sigma) to mimic the conditions present in the inflamed liver, or lipopolysaccharide (LPS, 100 ng/mL, Sigma) plus IFNγ (50 IU/mL, Peprotech) to produce a maximal macrophage activation. Day 7 macrophages are incubated overnight and supernatants collected and spun down to remove debris, then stored at −80° C. until testing. Secretome analysis is performed using a 27-plex human cytokine kit and a 9-plex matrix metalloprotease kit run on a Magpix multiplex enzyme linked immunoassay plate reader (BioRad).

### Product Stability:

Various excipients are tested during process development including PBS/EDTA buffer; PBS/EDTA buffer with 0.5% HAS (Alburex), 0.9% saline alone or saline with 0.5% HAS. The 0.9% saline (Baxter) with 0.5% HAS excipient is found to maintain optimal cell viability and phenotype (data not shown). The stability of the macrophages from cirrhotic donors after harvest is investigated in three process optimization runs, and a more limited range of time points assessed in the process validation runs (n=3). After harvest and re-suspension in excipient (0.9% saline for infusion, 0.5% human serum albumin), the bags are stored at ambient temperature (21-22° C.) and samples taken at 0, 2, 4, 6, 8, 12, 24, 30 and 48 h postharvest. The release criteria antibody panel is run on each sample, and viability and mean fold change from day 0 is measured from geometric MFI of 25F9 and CD206.

### Statistical Analysis:

Results are expressed as mean±SD. The statistical significance of differences is assessed where possible with the unpaired two-tailed t-test using GraphPad Prism 6. Results are considered statistically significant when the P value is <0.05.

### Example 3. CD5-FcR-PI3K CFP Construct

In this example, a CD5-targeted CFP was constructed using known molecular biology techniques. The CFP has an extracellular domain comprising a signal peptide fused to an scFv containing a heavy chain variable domain linked to a light chain variable domain that binds to CD5 on a target cell, attached to a CD8α chain hinge and CD8α chain TM domain via a short linker. The TM domain is fused at the cytosolic end with an FcRγ cytosolic portion, and a PI3K recruitment domain. The construct was prepared in a vector having a fluorescent marker and a drug (ampicillin) resistance and amplified by transfecting a bacterial host. The sequence is provided below:

CD5-FCR-PI3K
(SEQ ID NO: 34)
MWLQSLLLLGTVACSISEIQLVQSGGGLVKPGGSVRISCAASGYTFTNYG
MNWVRQAPGKGLEWMGWINTHTGEPTYADSFKGRFTFSLDDSKNTAYLQI
NSLRAEDTAVYFCTRRGYDWYFDVWGQGTTVTVSSGGGGSGGGGSGGGGS
DIQMTQSPSSLSASVGDRVTITCRASQDINSYLSWFQQKPGKAPKTLIYR
ANRLESGVPSRFSGSGSGTDYTLTISSLQYEDFGIYYCQQYDESPWTFGG
GTKLEIKSGGGGSGALSNSIMYFSHFVPVFLPAKPTTTPAPRPPTPAPTI
ASQPLSLRPEACRPAAGGAVHTRGLDIYIWAPLAGTCGVLLLSLVITLYC
RRLKIQVRKAAITSYEKSDGVYTGLSTRNQETYETLKHEKPPQGSGSYED
MRGILYAAPQLRSIRGQPGPNHEEDADSYENM.

mRNA was generated by in vitro transcription using linearized plasmids. The purified mRNA was electroporated into a cell line for expression analysis.

### Example 4. Plasmid Encoding CD5-FcR-PI3K CFP mRNA with Desired 5′- and 3′-UTRs

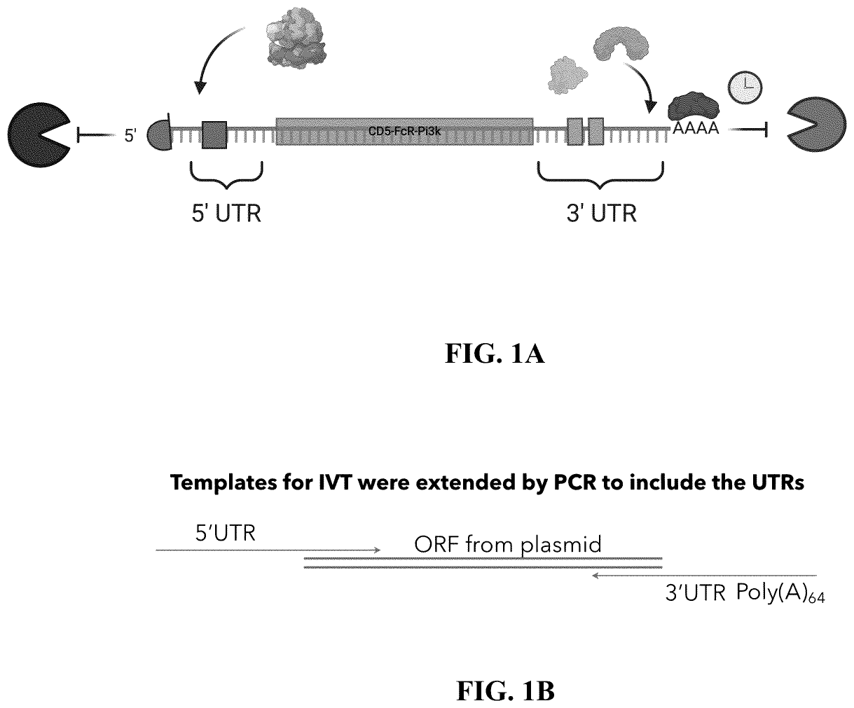

In this section a plasmid encoding CD5-FcR-PI3K mRNA is generated on the pcDNA3p1 backbone. The mRNA encoded by the plasmid is depicted graphically in FIG. 1A. Expression of the CFP is driven by a T7 promoter (TAATACGACTCACTATA) (SEQ ID NO: 35). The CD5-FcR-PI3K coding region is depicted in SEQ ID NO: 37 below. A sequence encoding a C3-5′ UTR is inserted having the sequence: GGGactcctccccatcctctccctctgtccctctgtccctctgaccctgcactgtcccagcacc (SEQ ID NO. 36). A sequence encoding a ORM-1 3′ UTR is inserted having the sequence: Caggacacagccttggatcaggacagagacttgggggccatcctgcccctccaacccgacatgtgtacctcagctttttccctcacttgcatcaataaag cttctgtgtttggaacag (SEQ ID NO: 43). A poly A stretch is inserted between the 3′ UTR and the restriction site Hpa1 (gtt|aac).

In another example, templates for IVT were extended by PCR to include the UTRs as shown in FIG. 1B. The poly A tail can be added enzymatically to the mRNA.

CD5 FcR PI3K Coding region (open reading frame):
(SEQ ID NO: 37)
ATGTGGCTGCAGTCTCTGCTGCTGCTGGGAACAGTGGCCTGTAGCATCTC
TGAGATCCAGCTGGTTCAGTCTGGCGGCGGACTTGTGAAACCTGGCGGAT
CTGTCAGAATCAGCTGTGCCGCCAGCGGCTACACCTTCACCAACTACGGC
ATGAACTGGGTCCGACAGGCCCCTGGAAAAGGCCTTGAGTGGATGGGCTG
GATCAATACCCACACCGGCGAGCCAACCTACGCCGATAGCTTTAAGGGCA
GATTCACCTTCAGCCTGGACGACAGCAAGAACACCGCCTACCTGCAGATC
AACAGCCTGAGAGCCGAGGATACCGCCGTGTACTTCTGCACCAGAAGAGG
CTACGACTGGTACTTCGATGTGTGGGGCCAGGGCACCACAGTGACAGTTT
CTAGCGGAGGCGGAGGATCAGGTGGCGGTGGATCTGGCGGTGGTGGCTCT
GATATCCAGATGACACAGAGCCCTAGCAGCCTGTCTGCCTCTGTGGGCGA
TAGAGTGACCATCACCTGTAGAGCCAGCCAGGACATCAACAGCTACCTGA
GCTGGTTCCAGCAGAAGCCTGGCAAGGCCCCTAAGACACTGATCTACCGG
GCCAACAGACTGGAAAGCGGCGTGCCAAGCAGATTTTCTGGCAGCGGCTC
TGGCACCGACTACACCCTGACAATCAGCAGCCTGCAGTACGAGGACTTCG
GCATCTACTACTGCCAGCAGTACGACGAGAGCCCTTGGACATTTGGCGGA
GGCACCAAGCTGGAAATCAAATCAGGCGGCGGAGGAAGCGGAGCCCTGAG
CAATAGCATCATGTACTTCAGCCACTTCGTGCCCGTGTTTCTGCCCGCCA
AGCCTACAACAACACCCGCTCCTAGACCACCTACACCAGCTCCTACAATC
GCCAGCCAGCCTCTGTCTCTCAGACCTGAAGCCTGTAGACCTGCTGCAGG
CGGAGCTGTGCATACCAGAGGCCTGGATATCTACATTTGGGCCCCTCTGG
CTGGCACATGTGGTGTCCTGCTGCTGTCTCTGGTCATCACCCTGTACTGC
AGACGGCTGAAGATCCAAGTGCGGAAGGCCGCCATCACCAGCTACGAGAA
ATCTGATGGCGTGTACACCGGCCTGAGCACCCGGAATCAAGAGACATACG
AGACACTGAAGCACGAGAAGCCTCCACAAGGCAGCGGCAGCTATGAGGAC
ATGAGAGGCATTCTGTACGCCGCTCCTCAGCTGCGGTCTATCAGAGGCCA
ACCTGGACCTAACCACGAAGAGGACGCCGACTCCTACGAGAACATGTGA.

### Example 5. Plasmid Encoding pp65 mRNA with Desired 5′- and 3′-UTRs

The plasmid construct using the same method as in the previous section (Example 4) has the following sequences:

T7 promoter:
(SEQ ID NO: 35)
TAATACGACTCACTATA.
5′ UTR:
(SEQ ID NO. 36)
GGGactcctccccatcctctccctctgtccctctgtccctctgaccctgc
actgtcccagcacc.
Gp65 signal peptide:
(SEQ ID NO: 38)
ATGAGGGCCCTGTGGGTGCTGGGCCTCTGCTGCGTCCTGCTGACCTTCGG
GTCGGTCAGAGCTGACGATGAAGTTGATG
Linker:
(SEQ ID NO: 39)
TGGCCATTGGGGCC
Pp65 Coding region (ORF):
(SEQ ID NO: 40)
ATGATATCCGTACTGGGTCCCATTTCGGGGCACGTGCTGAAAGCCGTGTT
TAGTCGCGGCGATACGCCGGTGCTGCCGCACGAGACGCGACTCCTGCAGA
CGGGTATCCACGTACGCGTGAGCCAGCCCTCGCTGATCTTGGTATCGCAG
TACACGCCCGACTCGACGCCATGCCACCGCGGCGACAATCAGCTGCAGGT
GCAGCACACGTACTTTACGGGCAGCGAGGTGGAGAACGTGTCGGTCAACG
TGCACAACCCCACGGGCCGAAGCATCTGCCCCAGCCAGGAGCCCATGTCG
ATCTATGTGTACGCGCTGCCGCTCAAGATGCTGAACATCCCCAGCATCAA
CGTGCACCACTACCCGTCGGCGGCCGAGCGCAAACACCGACACCTGCCCG
TAGCTGACGCTGTGATTCACGCGTCGGGCAAGCAGATGTGGCAGGCGCGT
CTCACGGTCTCGGGACTGGCCTGGACGCGTCAGCAGAACCAGTGGAAAGA
GCCCGACGTCTACTACACGTCAGCGTTCGTGTTTCCCACCAAGGACGTGG
CACTGCGGCACGTGGTGTGCGCGCACGAGCTGGTTTGCTCCATGGAGAAC
ACGCGCGCAACCAAGATGCAGGTGATAGGTGACCAGTACGTCAAGGTGTA
CCTGGAGTCCTTCTGCGAGGACGTGCCCTCCGGCAAGCTCTTTATGCACG
TCACGCTGGGCTCTGACGTGGAAGAGGACCTGACGATGACCCGCAACCCG
CAACCCTTCATGCGCCCCCACGAGCGCAACGGCTTTACGGTGTTGTGTCC
CAAAAATATGATAATCAAACCGGGCAAGATCTCGCACATCATGCTGGATG
TGGCTTTTACCTCACACGAGCATTTTGGGCTGCTGTGTCCCAAGAGCATC
CCGGGCCTGAGCATCTCAGGTAACCTGTTGATGAACGGGCAGCAGATCTT
CCTGGAGGTACAAGCCATACGCGAGACCGTGGAACTGCGTCAGTACGATC
CCGTGGCTGCGCTCTTCTTTTTCGATATCGACTTGCTGCTGCAGCGCGGG
CCTCAGTACAGCGAGCACCCCACCTTCACCAGCCAGTATCGCATCCAGGG
CAAGCTTGAGTACCGACACACCTGGGACCGGCACGACGAGGGTGCCGCCC
AGGGCGACGACGACGTCTGGACCAGCGGATCGGACTCCGACGAAGAACTC
GTAACCACCGAGCGCAAGACGCCCCGCGTCACCGGCGGCGGCGCCATGGC
GGGCGCCTCCACTTCCGCGGGCCGCAAACGCAAATCAGCATCCTCGGCGA
CGGCGTGCACGTCGGGCGTTATGACACGCGGCCGCCTTAAGGCCGAGTCC
ACCGTCGCGCCCGAAGAGGACACCGACGAGGATTCCGACAACGAAATCCA
CAATCCGGCCGTGTTCACCTGGCCGCCCTGGCAGGCCGGCATCCTGGCCC
GCAACCTGGTGCCCATGGTGGCTACGGTTCAGGGTCAGAATCTGAAGTAC
CAGGAATTCTTCTGGGACGCCAACGACATCTACCGCATCTTCGCCGAATT
GGAAGGCGTATGGCAGCCCGCTGCGCAACCCAAACGTCGCCGCCACCGGC
AAGACGCCTTGCCCGGGCCATGCATCGCCTCGACGCCCAAAAAGCACCGA
GGT
Linker:
(SEQ ID NO: 41)
GGCCTTGTTGGCC
LAMP sorting sequence:
(SEQ ID NO: 42)
TGATCCCCATCGCTGTGGGTGGTGCCCTGGCGGGGCTGGTCCTCATCGTC
CTCATCGCCTACCTCGTCGGCAGGAAGAGGAGTCACGCAGGCTACCAGAC
TATCTAG
3′ UTR:
(SEQ ID NO: 43)
Caggacacagccttggatcaggacagagacttgggggccatcctgcccct
ccaacccgacatgtgtacctcagctttttccctcacttgcatcaataaag
cttctgtgtttggaacag

### Restriction Site Used is HpaI

### Example 6. HER2-FcR-PI3K CFP Construct

In this example, a HER2-targeted CFP was constructed using known molecular biology techniques. The CFP has an extracellular domain comprising a signal peptide fused to an scFv containing a heavy chain variable domain linked to a light chain variable domain that binds to HER2 on a target cell, attached to a CD8α chain hinge and CD8α chain TM domain via a short linker. The TM domain is fused at the cytosolic end with an FcRγ cytosolic portion, and a PI3K recruitment domain as in the previous example. The sequence is provided below:

HER2-FCR-PI3K
(SEQ ID NO: 44)
MWLQSLLLLGTVACSISDIQMTQSPSSLSASVGDRVTITCRASQDVNTAV
AWYQQKPGKAPKLLIYSASFLYSGVPSRFSGSRSGTDFTLTISSLQPEDF
ATYYCQQHYTTPPTFGQGTKVEIKRTGSTSGSGKPGSGEGSEVQLVESGG
GLVQPGGSLRLSCAASGFNIKDTYIHWVRQAPGKGLEWVARIYPTNGYTR
YADSVKGRFTISADTSKNTAYLQMNSLRAEDTAVYYCSRWGGDGFYAMDV
WGQGTLVTVSSSGGGGSGALSNSIMYFSHFVPVFLPAKPTTTPAPRPPTP
APTIASQPLSLRPEACRPAAGGAVHTRGLDIYIWAPLAGTCGVLLLSLVI
TLYCRRLKIQVRKAAITSYEKSDGVYTGLSTRNQETYETLKHEKPPQGSG
SYEDMRGILYAAPQLRSIRGQPGPNHEEDADSYENM.

### Example 7. CD5-FcR-CD40 CFP Construct

In this example, a CD5-targeted CFP was constructed using known molecular biology techniques having an intracellular domain comprising CD40 sequence. The CFP has an extracellular domain comprising a signal peptide fused to an scFv containing a heavy chain variable domain linked to a light chain variable domain that binds to CD5 on a target cell, attached to a CD8α chain hinge and CD8α chain TM domain via a short linker. The TM domain is fused at the cytosolic end with an FcRγ cytosolic portion, followed by a CD40 cytosolic portion. The sequence is provided below:

CD5-FcR-CD40
(SEQ ID NO: 45)
MWLQSLLLLGTVACSISEIQLVQSGGGLVKPGGSVRISCAASGYTFTNYG
MNWVRQAPGKGLEWMGWINTHTGEPTYADSFKGRFTFSLDDSKNTAYLQI
NSLRAEDTAVYFCTRRGYDWYFDVWGQGTTVTVSSGGGGSGGGGSGGGGS
DIQMTQSPSSLSASVGDRVTITCRASQDINSYLSWFQQKPGKAPKTLIYR
ANRLESGVPSRFSGSGSGTDYTLTISSLQYEDFGIYYCQQYDESPWTFGG
GTKLEIKSGGGGSGALSNSIMYFSHFVPVFLPAKPTTTPAPRPPTPAPTI
ASQPLSLRPEACRPAAGGAVHTRGLDIYIWAPLAGTCGVLLLSLVITLYC
RLKIQVRKAAITSYEKSDGVYTGLSTRNQETYETLKHEKPPQKKVAKKPT
NKAPHPKQEPQEINFPDDLPGSNTAAPVQETLHGCQPVTQEDGKESRISV
QERQ.

### Example 8. Expression of Anti-CD5 and Anti-HER2 CFPs

In this example, cells from monocytic cell line THP-1, are electroporated with individual anti-CD5 CFP (CD5 CAR) constructs with either no intracellular domain (No ICD); or intracellular domain (ICD) having a CD40 signaling domain, or a FcR signaling domain; or with PI3kinase (PI3K) recruitment signaling domain; or with a first CD40 signaling domain and a second signaling domain from FcRγ intracellular domain or vice versa; with a first FcRγ signaling domain and a second PI3K recruitment signaling domain or vice versa, and expression of the CAR construct was determined by flow cytometry using antibody targeted to the extracellular domain. The following table shows the various CFP constructs with combinations of domains used in the study.

TABLE 2
CD5-CFP constructs and HER2 CFP constructs

| | Intracellular | TM | Extracellular | Antigen binding |
|---|---|---|---|---|
| Construct Name | domain | domain | domain | domain |
| CD5-CD8h-CD8tm-CD40-FcR | CD40 and FcRγ | CD8 | CD8 | Anti-CD5 scFv |
| CD5-CD8h-CD8tm-FcR-CD40 | FcRγ and CD40 | CD8 | CD8 | Anti-CD5 scFv |
| CD5-CD8h-CD8tm-FcR-PI3K | FcRγ and PI3K | CD8 | CD8 | Anti-CD5 scFv |
| CD5-CD8h-CD8tm-FcR | FcRγ | CD8 | CD8 | Anti-CD5 scFv |
| CD5-CD8h-CD8tm-no ICD | None | CD8 | CD8 | Anti-CD5 scFv |
| CD5-CD28h-CD28tm-FcR-PI3K | FcRγ and PI3K | CD28 | CD28 | Anti-CD5 scFv |
| CD5-CD8h-CD68tm-FcR-PI3K | FcRγ and PI3K | CD68 | CD8 | Anti-CD5 scFv |
| CD5-CD8tm-FcR-PI3K | FcRγ and PI3K | CD8 | — | Anti-CD5 scFv |
| CD5-CD28tm-FcR-PI3K | FcRγ and PI3K | CD28 | — | Anti-CD5 scFv |
| CD5-CD68tm-FcR-PI3K | FcRγ and PI3K | CD68 | — | Anti-CD5 scFv |
| CD5-CD8h-CD8tm-FcR-TNFR1 | FcγR and TNFR2 | CD8 | CD8 | Anti-CD5 scFv |
| CD5-CD8h-CD8tm-FcR-TNFR2 | FcRγ and TNFR2 | CD8 | CD8 | Anti-CD5 scFv |
| HER2-CD8h-CD8tm-CD40-FcR | CD40 and FcRγ | CD8 | CD8 | Anti-HER2 scFv |
| HER2-CD8h-CD8tm-FcR-CD40 | FcRγ and CD40 | CD8 | CD8 | Anti-HER2 scFv |
| HER2-CD8h-CD8tm-FcR-PI3K | FcRγ and PI3K | CD8 | CD8 | Anti-HER2 scFv |
| HER2-CD8h-CD8tm-FcR | FcRγ | CD8 | CD8 | Anti-HER2 scFv |
| HER2-CD8h-CD8tm-no ICD | None | CD8 | CD8 | Anti-HER2 scFv |
| HER2-CD28h-CD28tm-FcR-PI3K | FcRγ and PI3K | CD28 | CD28 | Anti-HER2 scFv |
| HER2-CD8h-CD68tm-FcR-PI3K | FcRγ and PI3K | CD68 | CD8 | Anti-HER2 scFv |
| HER2-CD8tm-FcR-PI3K | FcRγ and PI3K | CD8 | — | Anti-HER2 scFv |
| HER2-CD28tm-FcR-PI3K | FcRγ and PI3K | CD28 | — | Anti-HER2 scFv |
| HER2-CD68tm-FcR-PI3K | FcRγ and PI3K | CD68 | — | Anti-HER2 scFv |
| HER2-CD8h-CD8tm-FcR-TNFR1 | FcRγ and TNFR2 | CD8 | CD8 | Anti-HER2 scFv |
| HER2-CD8h-CD8tm-FcR-TNFR2 | FcRγ and TNFR2 | CD8 | CD8 | Anti-HER2 scFv |

### Example 9. Phagocytosis and Activation Assays

For functional analysis of the various anti-CD5 CFP expressing THP-1 macrophages, cells are fed 6 μm FITC-labeled CD5 antigen-coated beads and phagocytic engulfment of the FITC beads per cell is quantitated by flow cytometry. Control beads are BSA coated. Experimental CD5-coated beads were readily engulfed by THP-1 cells. Each of the constructs show high level of phagocytosis that is target specific, and the CD5-coated bead uptake is higher compared to uptake of BSA coated beads.

Primary monocytes electroporated with the anti-CD5-CAR construct are assayed for bead engulfment, target specificity and cytokine as above. With pHRodo labeled target cells, increased phagocytic engulfment is noticed in case of any of the monocytic cells expressing any of the CD5-binder constructs, compared to mock electroporated cells. In another experiment, primary monocytes were electroporated with an anti-CD5-CAR construct (CD5-CD8h-CD8tm-FcR-PI3K) and assayed for phagocytosis and cytokine release.

### Example 10. Improved Expression of CFP Constructs with Various 5′ and 3′ UTRs

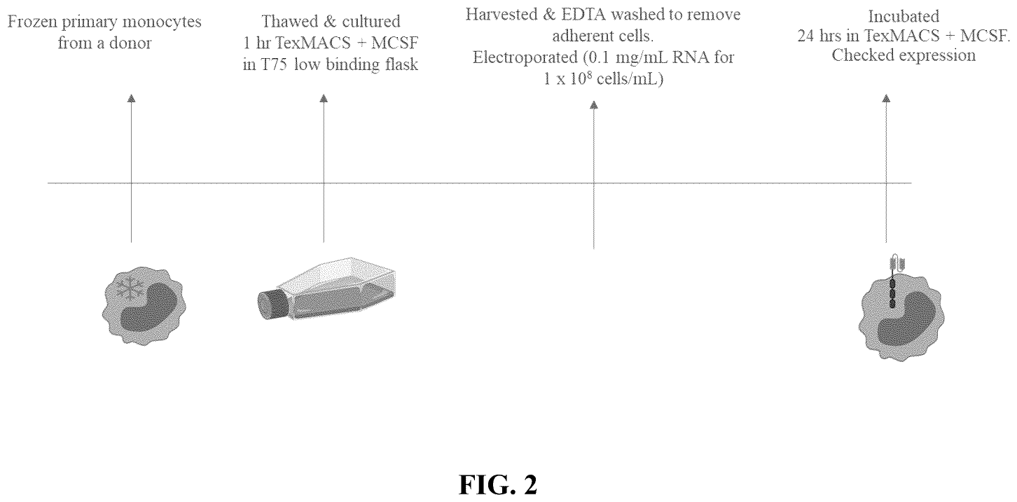

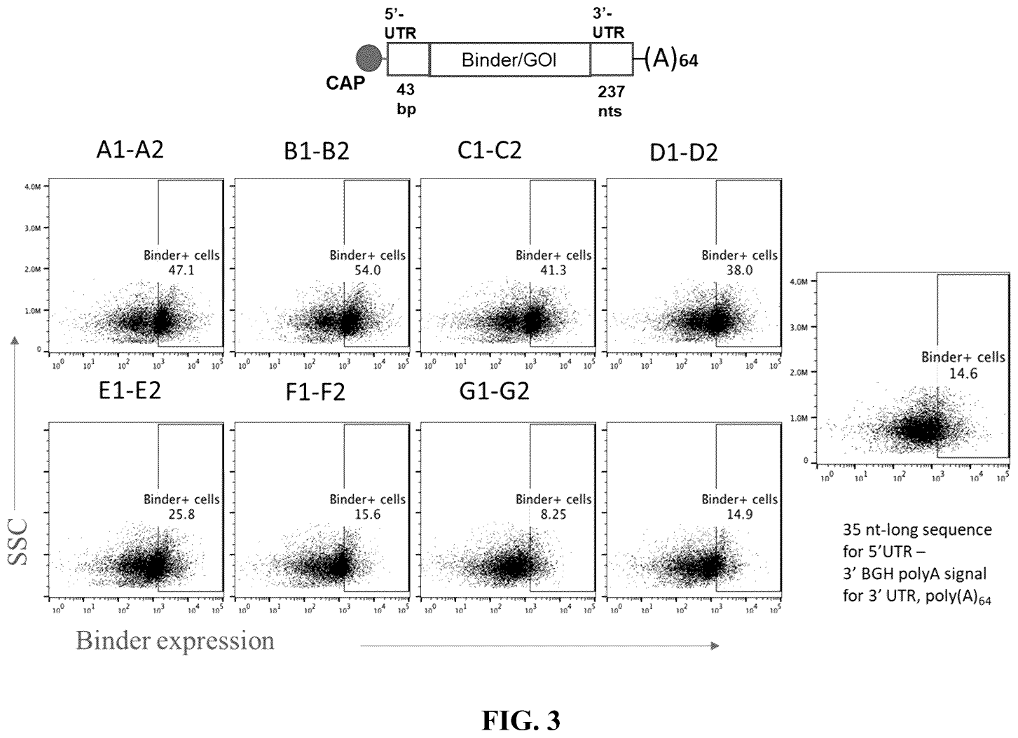

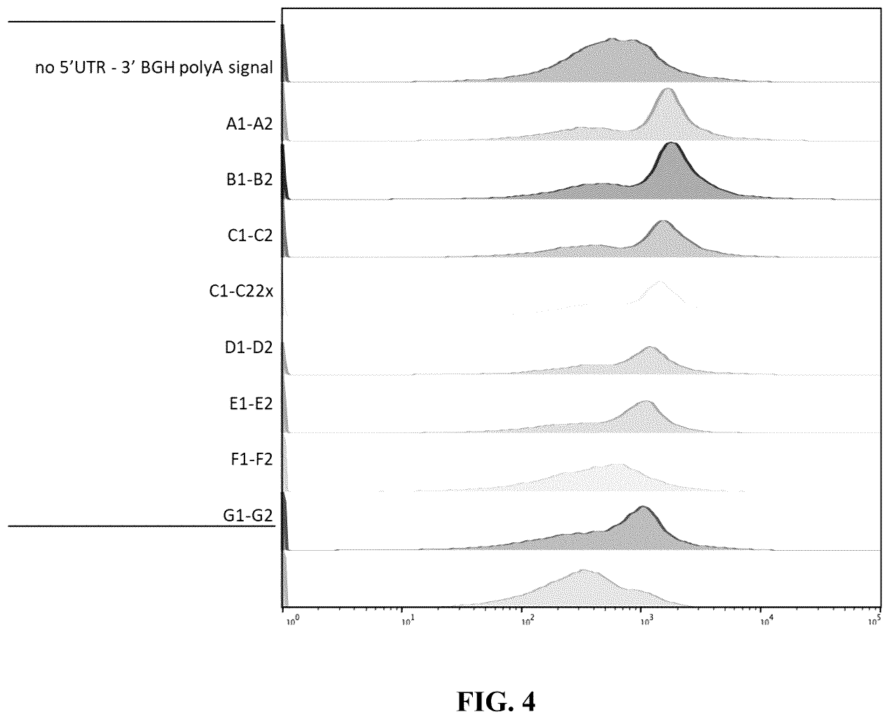

In this example, the effect of different UTRs on expression of an mRNA encoding a protein was investigated. Exemplary mRNA encoding a CCD5 CFP is depicted in FIG. 1A, and in an exemplary method, plasmid encoded UTRs were used, templates were subjected to IVT and the mRNA was obtained for transfection into myeloid cells (e.g., via electroporation). Templates for IVT including an ORF encoding the protein were extended by PCR to include the UTRs (FIG. 1B). Myeloid cells (human) (CD14+/CD16−) previously isolated and frozen were thawed, cultured in low binding flasks and electroporated with either the CD5 binder plasmid constructs or HER2 binder constructs (i) either having a 35 nucleotide long 5′ UTR and a BGH 3′ UTR having a 64 nucleotide polyA tail, or (ii) modified UTR constructs having the various 5′- and 3′-UTR combinations as depicted in the diagram. 24 hours following electroporation, expression of the binder was determined by flow cytometry (FIG. 2). As shown in FIG. 3 and FIG. 4, drastic improvements in expression levels were noted using the A1-A2, B1-B2, C1-C2 as well as D1-D2 pairs compared to less than 15% using the 35 nucleotides 5′-UTR or nucleotides 5′ UTR and BGH 3′ UTR. B1-B2 combination shows the highest change in expression, with 54% cells positive for the binder. In C1-C2-2x, 2 copies of the beta-globin 3′-UTR is inserted.

### Example 11. Expression of Binder Constructs in Cells Electroporated with In Vitro Transcribed mRNA Having Enzymatically Added Poly a Tail Compared to Plasmid Encoded Poly a Tail

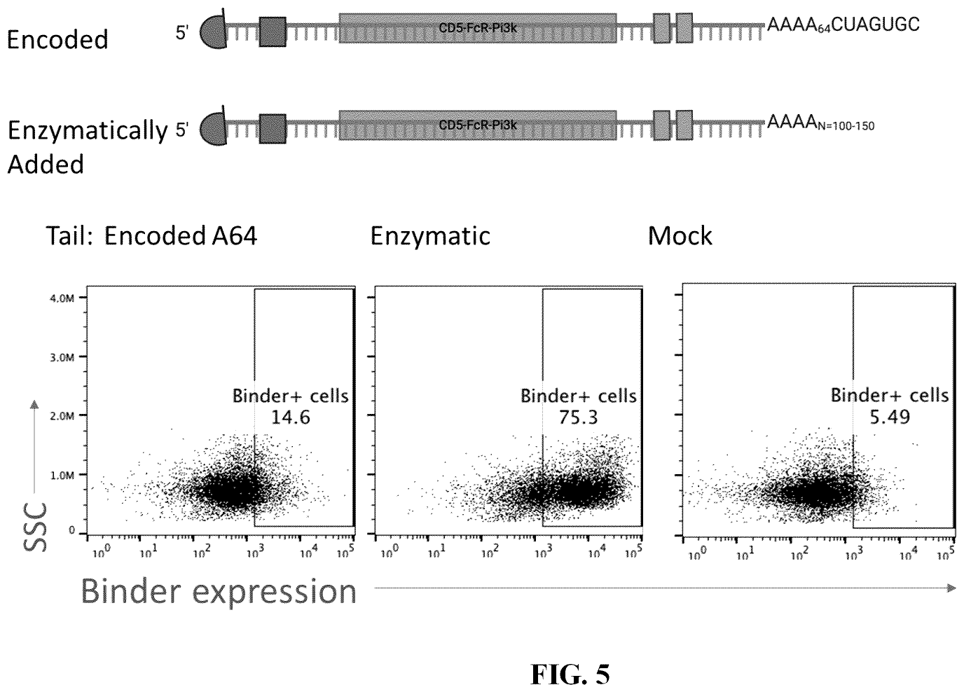

In this example, a surprisingly large enhancement of expression of the binder constructs is noted in myeloid cells (human) (CD14+/CD16−) when electroporated with CD5 binder mRNA constructs in which the poly A tail is enzymatically added. As in the previous experiment in Example 10, cells were previously isolated and frozen were thawed, cultured in low binding flasks and electroporated with the mRNA. Results are shown in FIG. 5. As shown in the figure, 75.3% cells expressed the mRNA encoded CFP with the CD5-binding extracellular domain when the mRNA was capped enzymatically, compared to much lower binder positive cells in the experimental set expressing the same mRNA but with a plasmid encoded poly A tail of 64 adenosine nucleotides. Results were obtained after thawing and culturing the cells for 24 hours.

### Example 12. Prolonged Expression of Binder Constructs with Enzymatically Added Poly a Tails

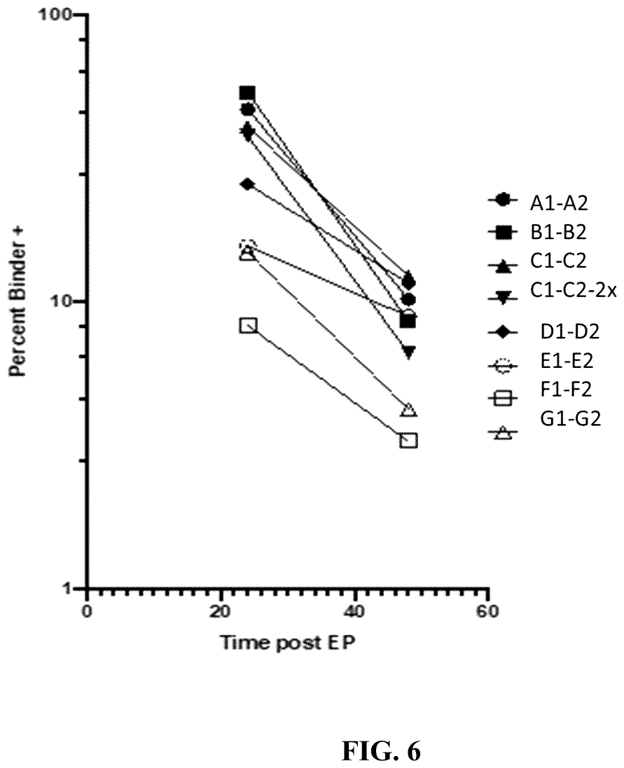

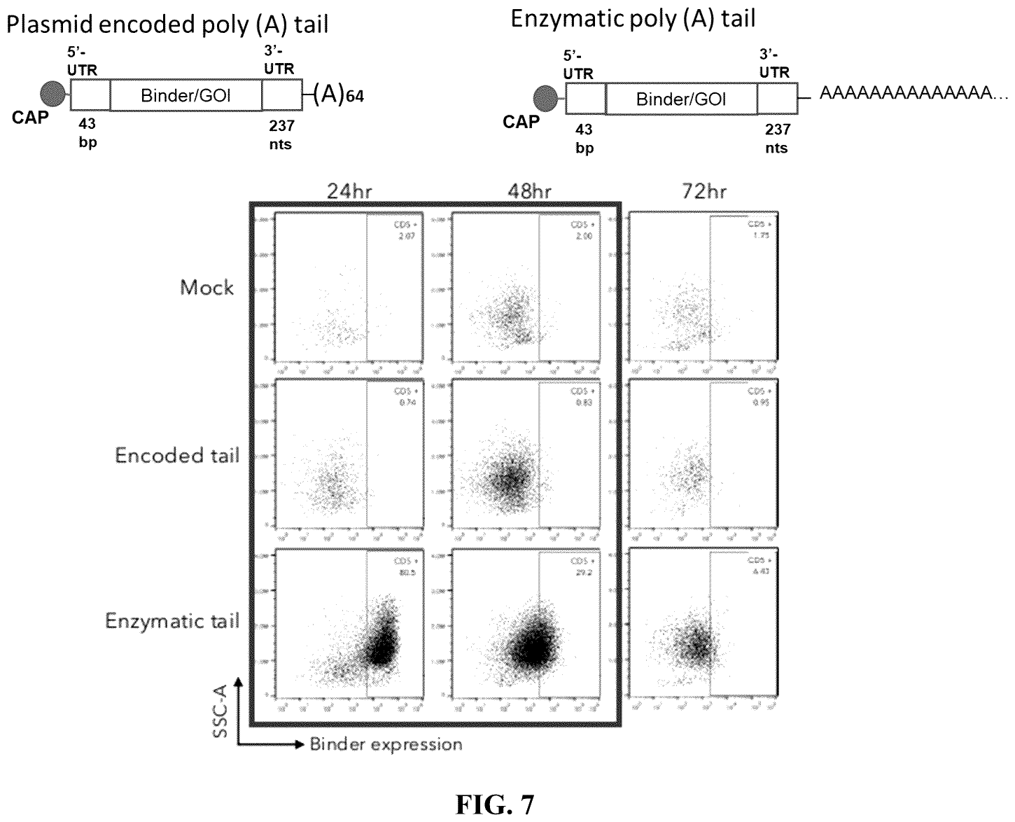

High expression of the constructs in which the poly A tail is enzymatically added were detected in the CD14/CD16− myeloid cells at 48 hours post electroporation (EP) (FIG. 6, time in hours). This indicates that the poly A tails added enzymatically confer high stability to the mRNA and continued expression of the CD5 binder protein. In another comparison study, in vitro transcribed RNA encoding a CD5-CFP (which could also be replaced with sequence encoding any gene of interest (GOI)), were electroporated in monocytes that were isolated and cultured for 24 hours or less, and cultured at 37° C. for various time points to test the expression of the mRNA encoded CFP. In this case, the mRNA construct either had a plasmid encoded poly A tail (A64), or enzymatically added poly A tail. The results shown in FIG. 7 further confirms that enzymatically added poly A tail confers longer duration of expression of the mRNA in primary monocytes, evident at 72 hours post electroporation.

### Example 13. Effect of Nucleotide Modifications on Expression of mRNA Encoded Proteins in THP-1 Cells

In this experiment, mRNA encoding a sample gene of interest, in this case a CD5-expressing CFP was variously modified or left unmodified (control) and tested for effect of the modifications on expression robustness and duration of the expression in myeloid cells, in this case a CD14+ cell. mRNA constructs were designed with the modifications detailed in Table 3.

TABLE 3
Nucleotide modifications

| Modified | Modification | Poly A poly- |
|---|---|---|
| nucleotides | location | merase source |
| Unmodified | None | E. coli |
| Phosphorothioates | Internal | E. coli |
| 2′ azido | Internal | Yeast |
| 3′ azido | 3′-Terminal | Yeast |

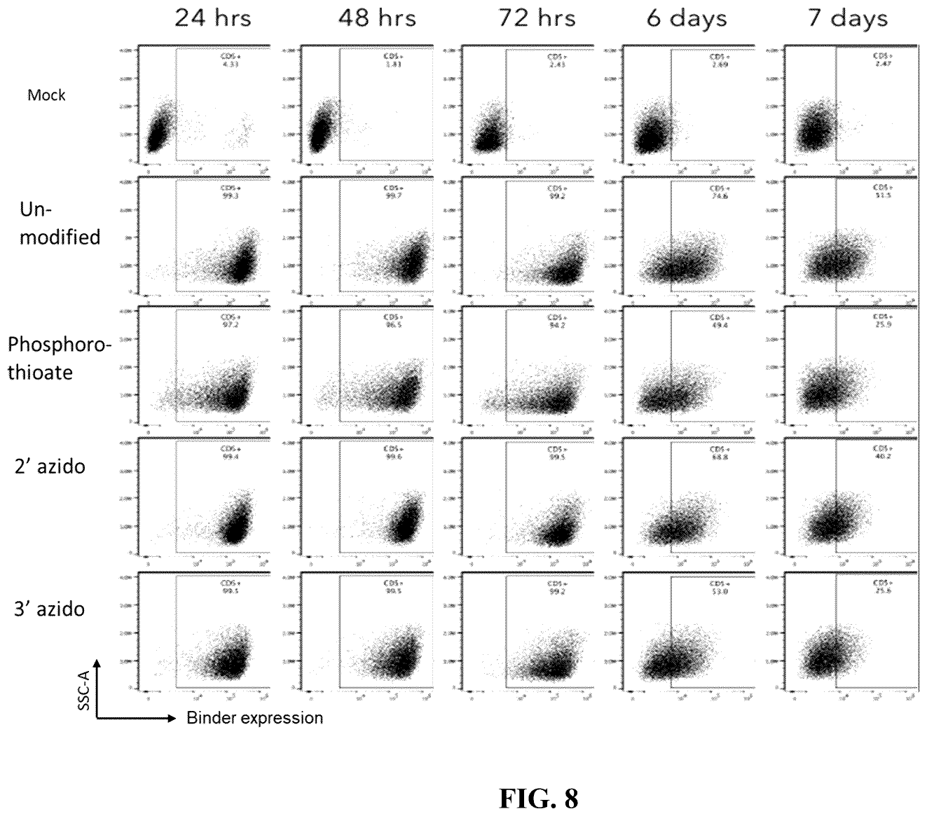

As shown in FIG. 8, nucleotide modifications did not significantly affect expression levels or durability of expression of mRNA encoded protein in THP-1 cells.

### Example 14. Effect of Different 5′-CAP Modifications on Expression of mRNA Encoded Proteins in Myeloid Cells

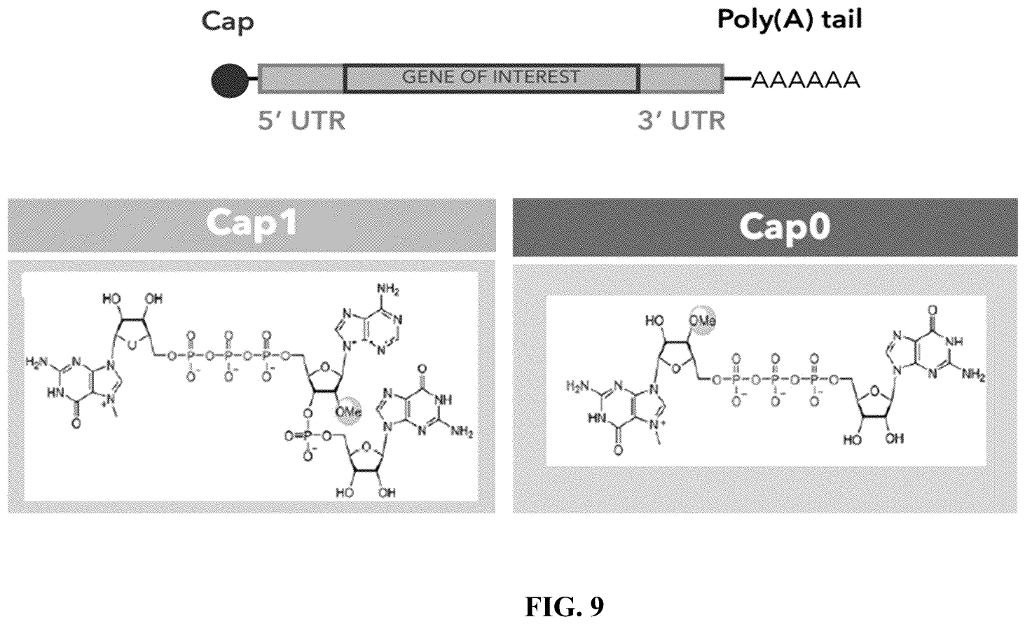

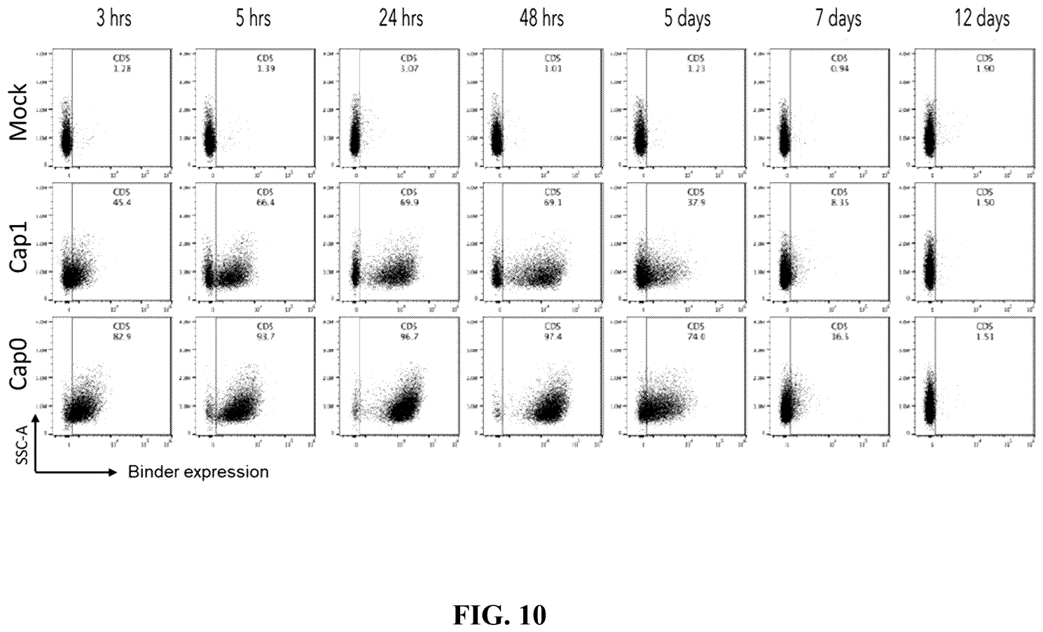

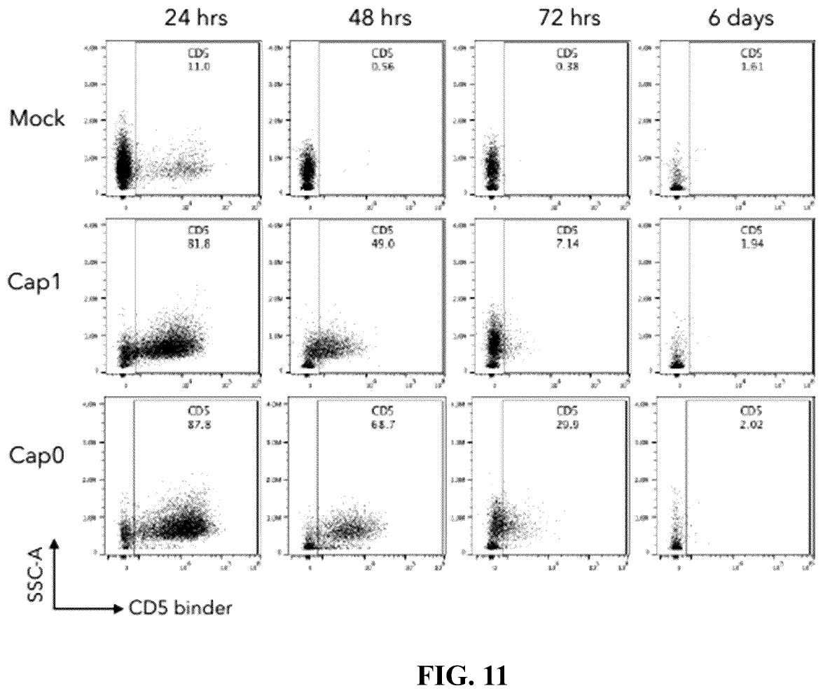

The effect of different methods of capping mRNA, as well as use of Cap1 versus Cap 0 (FIG. 9) was investigated. In this example, Cap 1 modification was performed by enzymatic capping using Vaccinia capping enzyme and 2′-O methyl transferase. The process steps involve generating the mRNA by in vitro transcription (IVT), followed by DNAse 1 treatment, tailing, enzymatic capping, and RNA purification. Capping efficiency was greater than 95%. Cap 0 structure (right) is introduced co-transcriptionally using anti-reverse cap analog (ARCA). The process steps involve generating the mRNA by in vitro transcription (IVT), followed by DNAse 1 treatment, tailing and RNA purification. Capping efficiency was 80%. Results shown in FIG. 10 indicate that the method of capping or type of cap had little effect on the mRNA expression or durability of the expression in THP-1 cells. Similar observation was made for human monocytes, shown in FIG. 11.

### Example 15. Effect of Use of Uridine Modified Nucleotides During IVT on mRNA Expression in Myeloid Cells

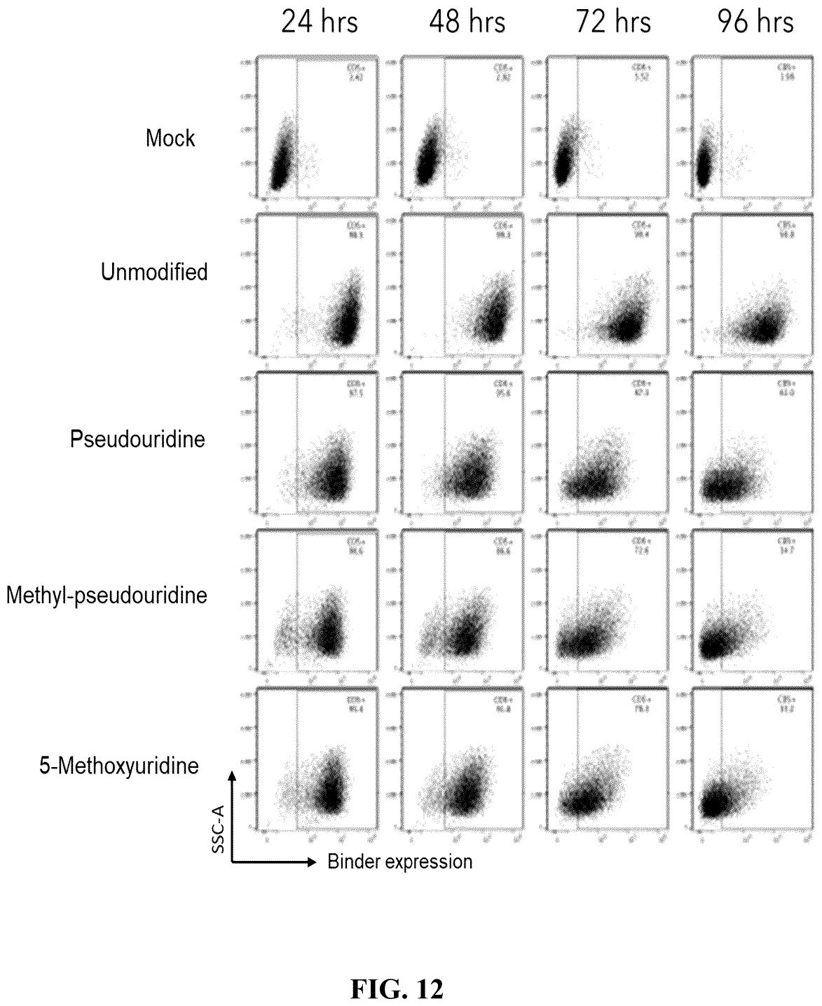

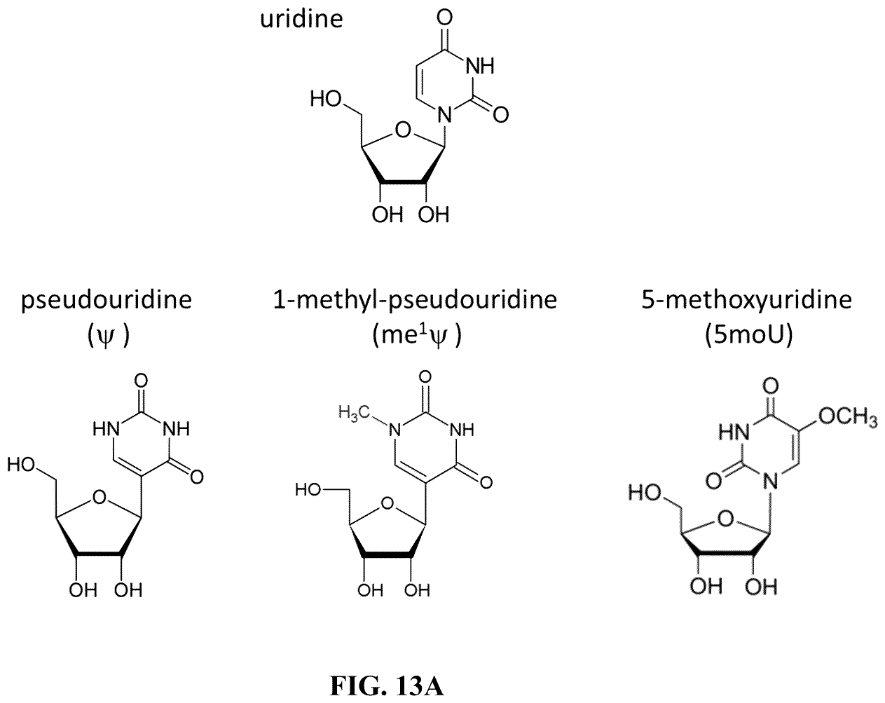

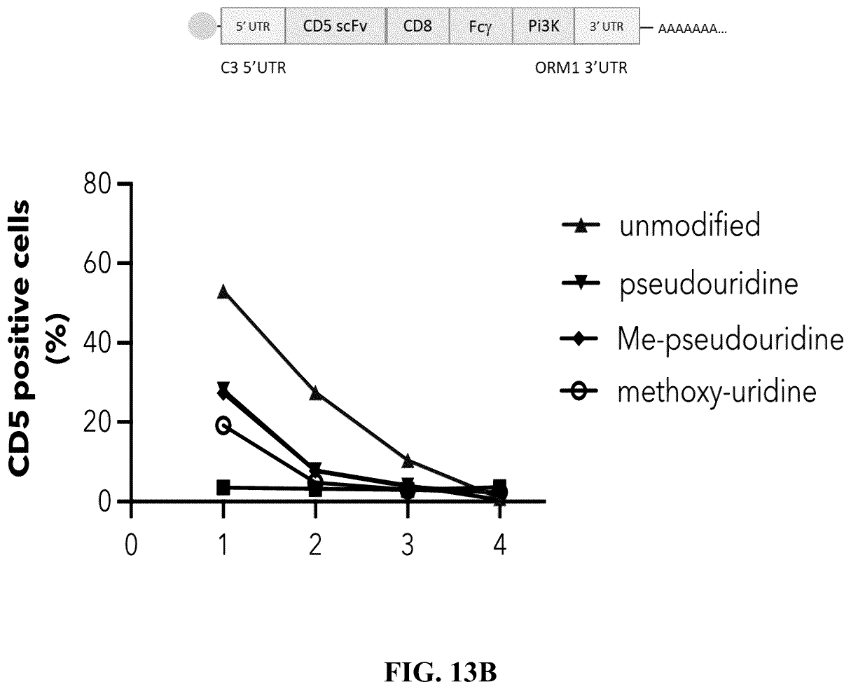

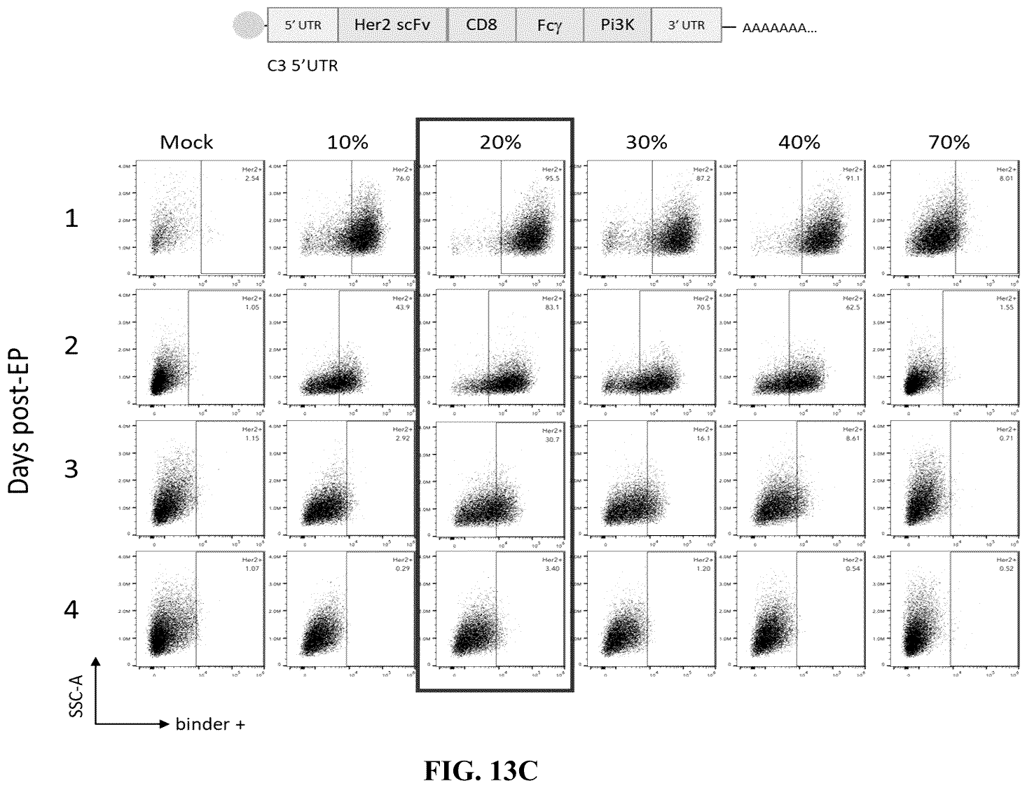

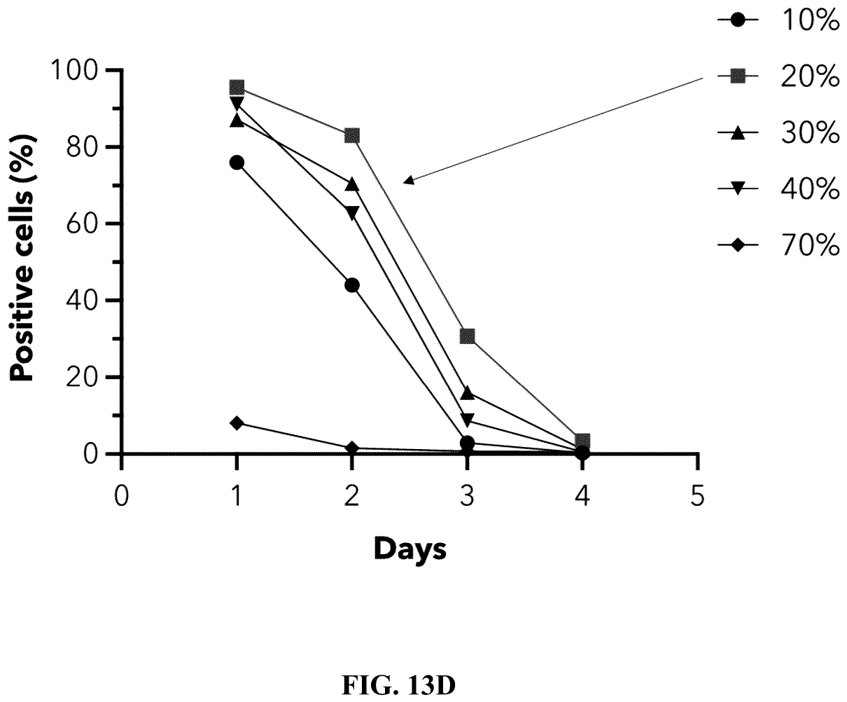

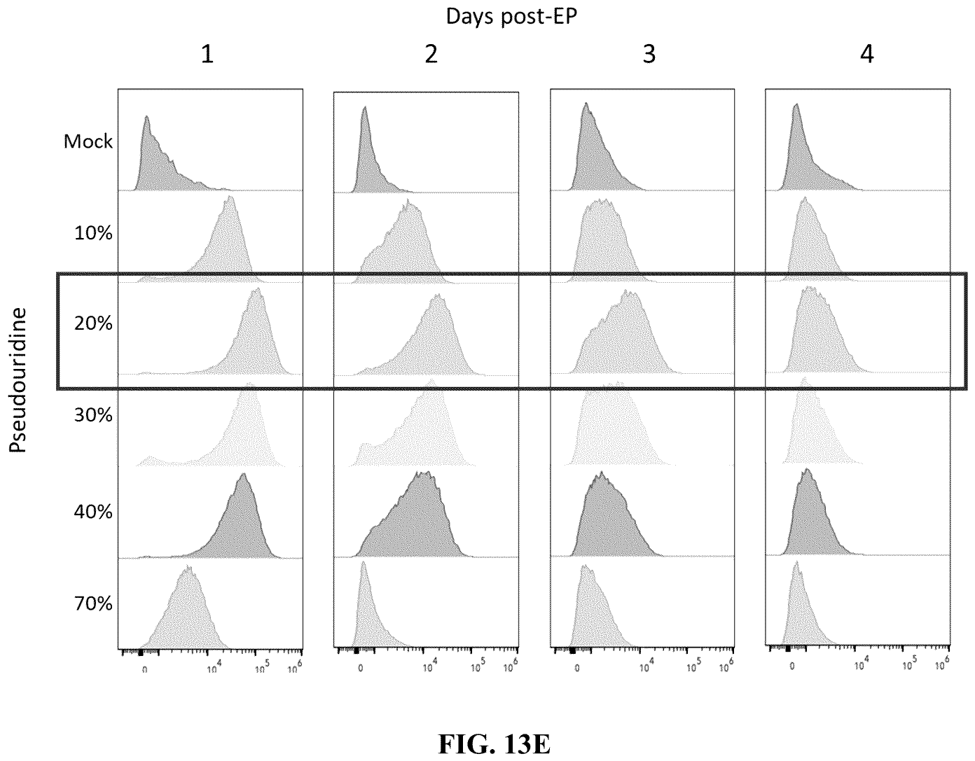

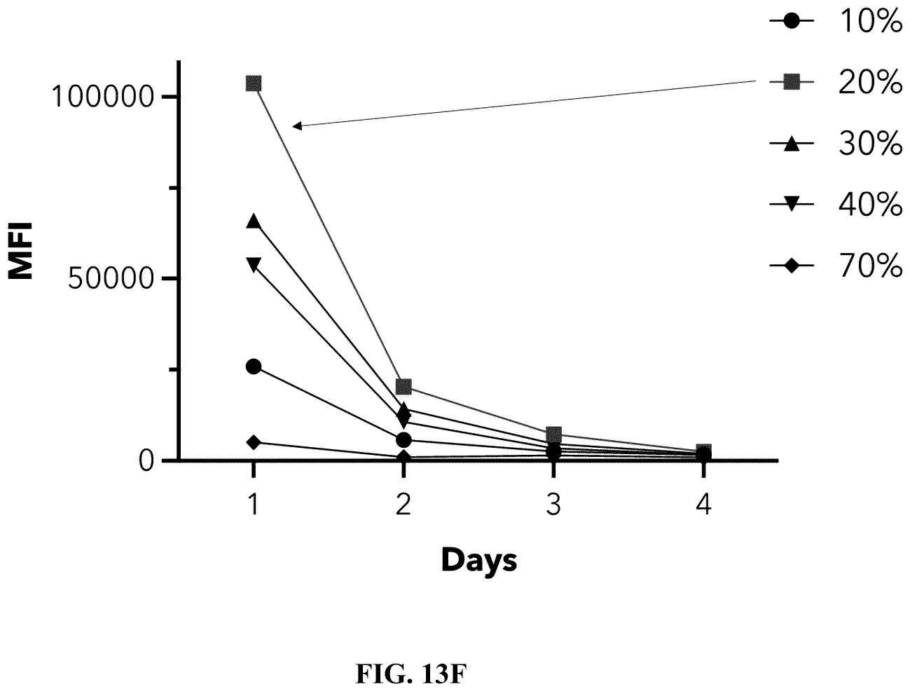

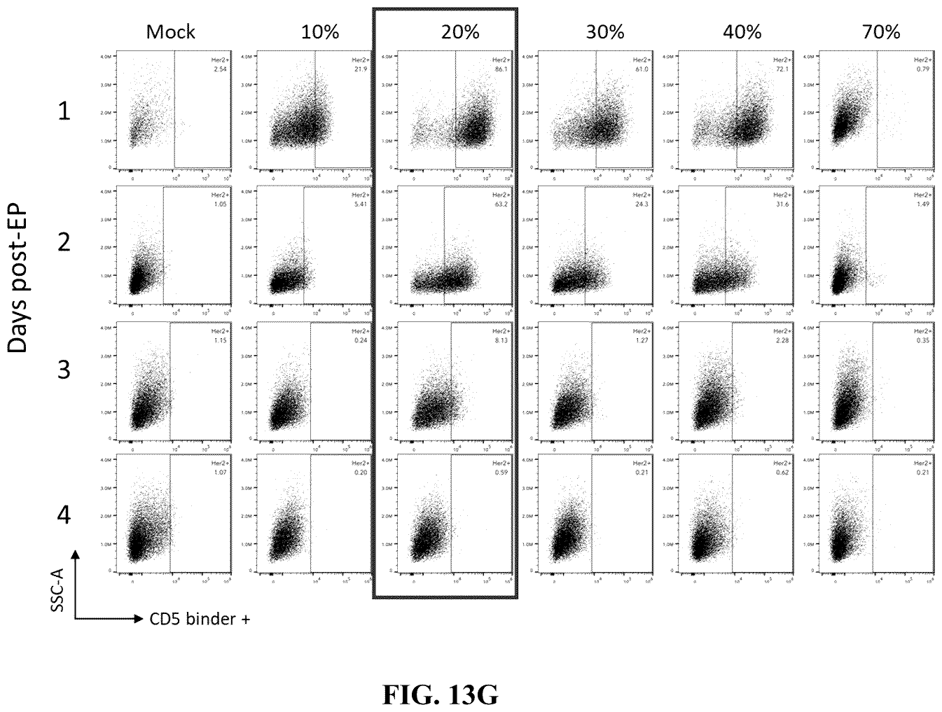

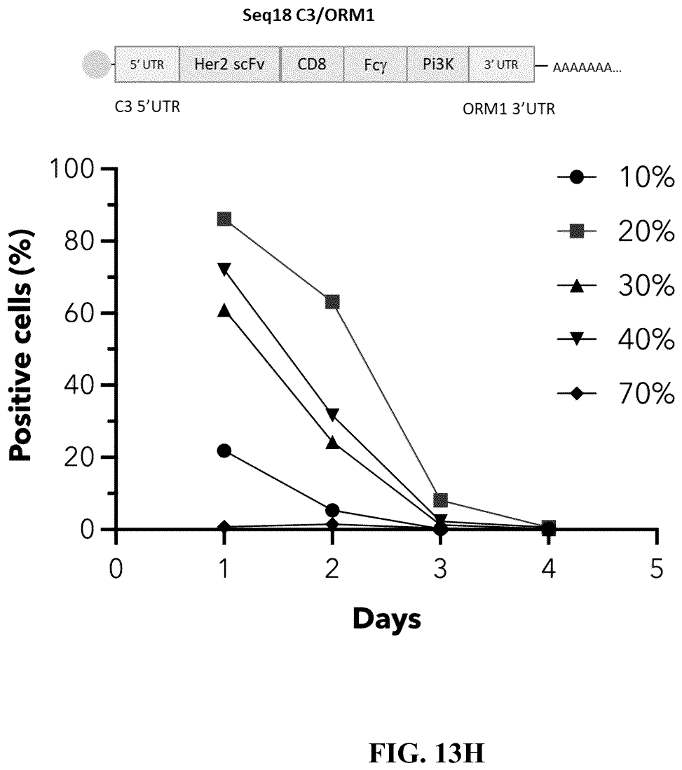

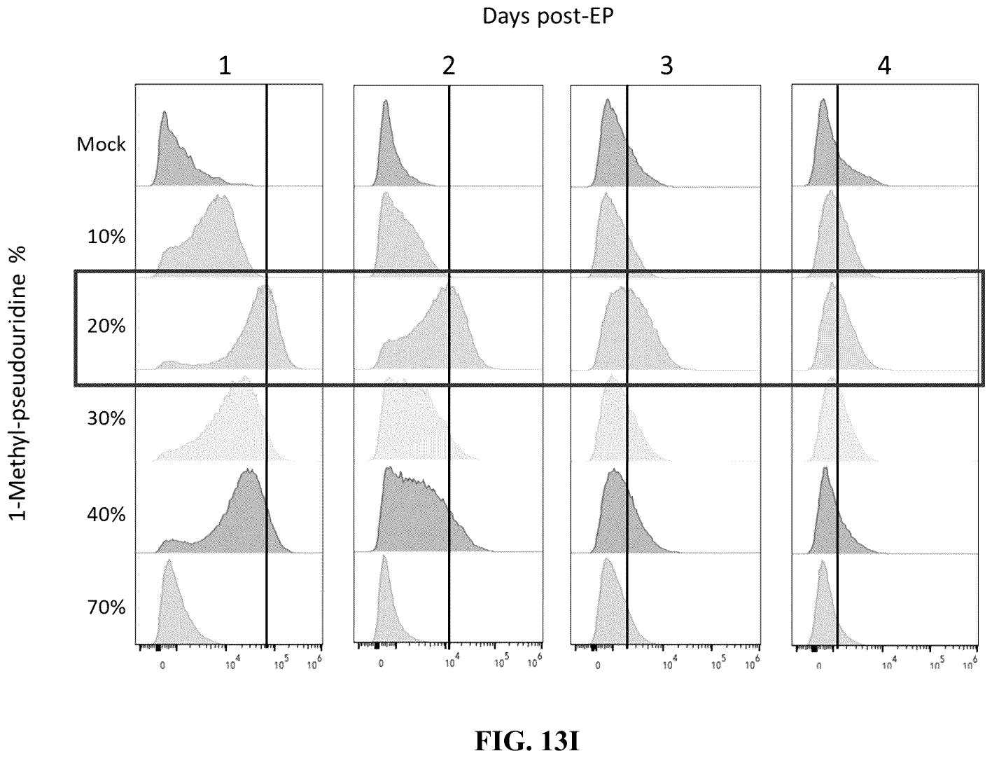

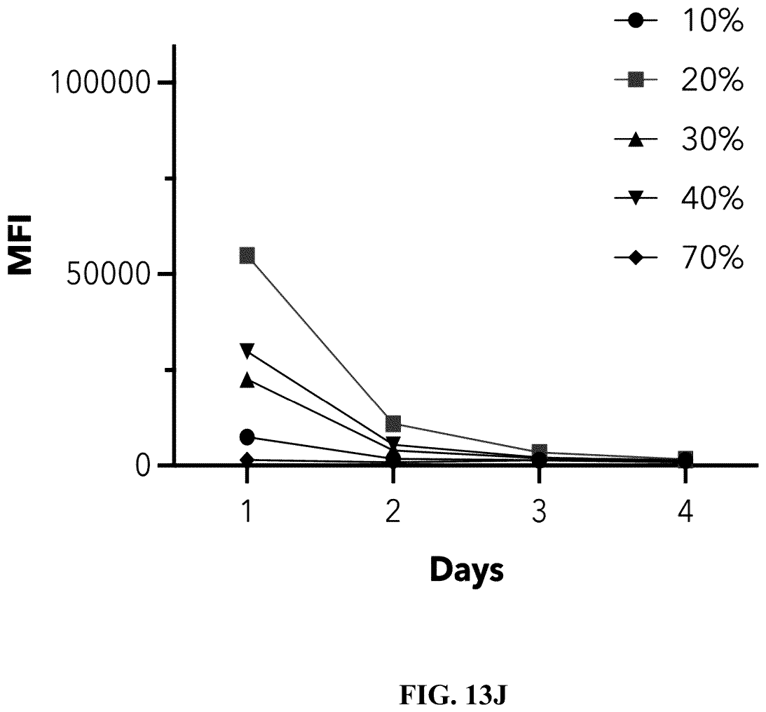

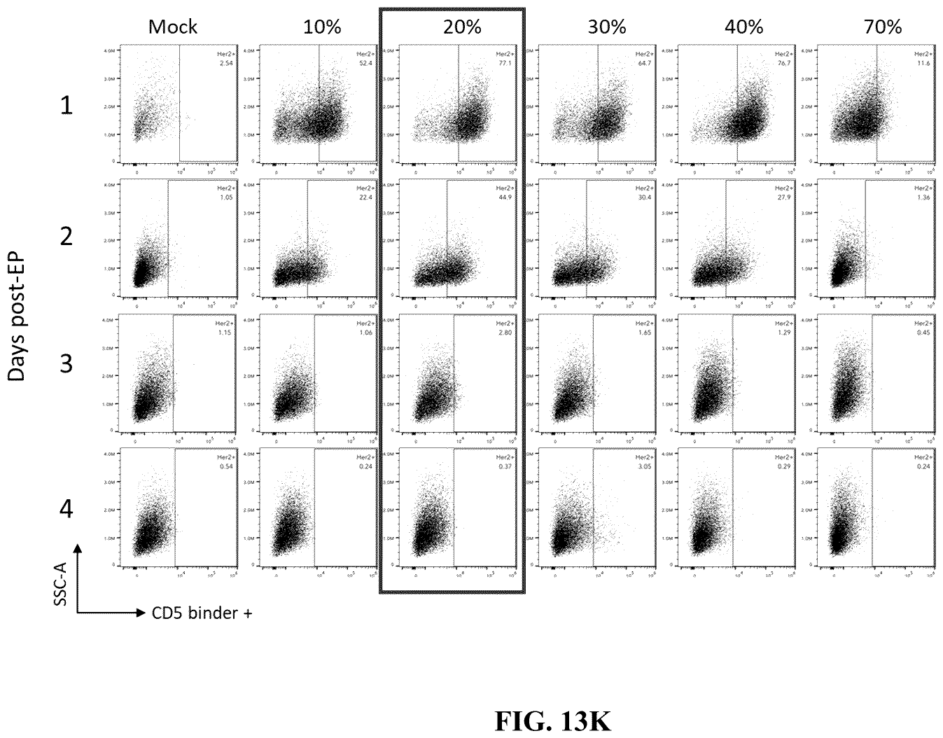

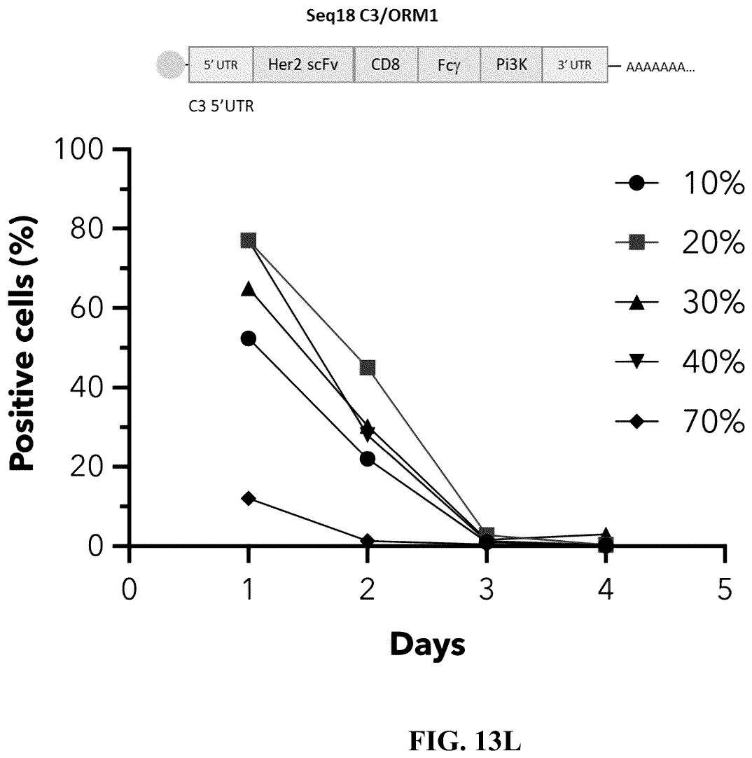

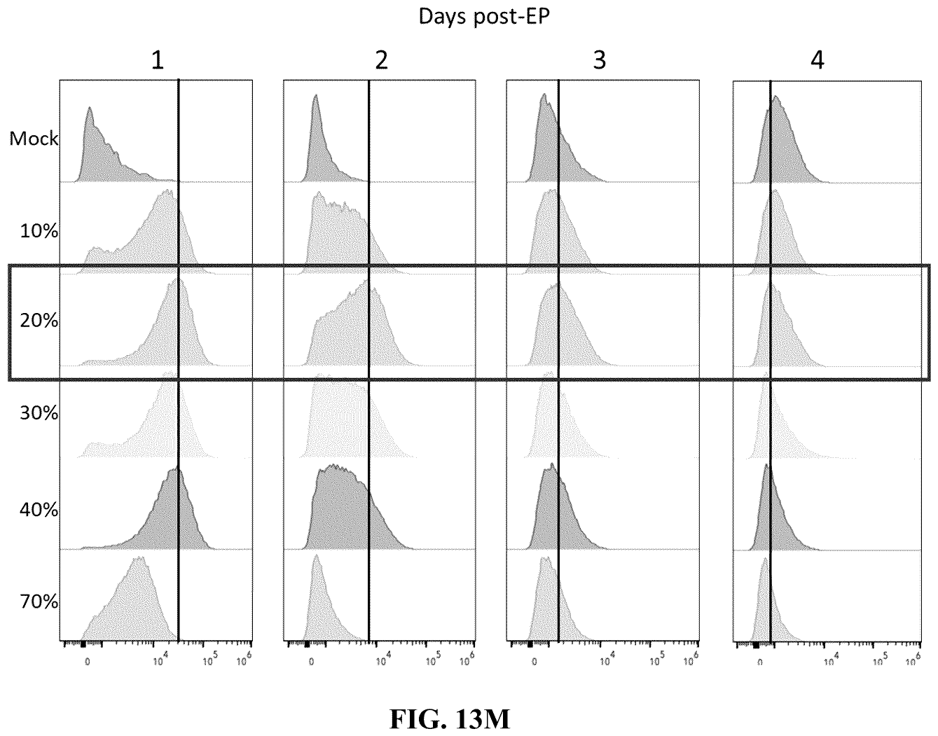

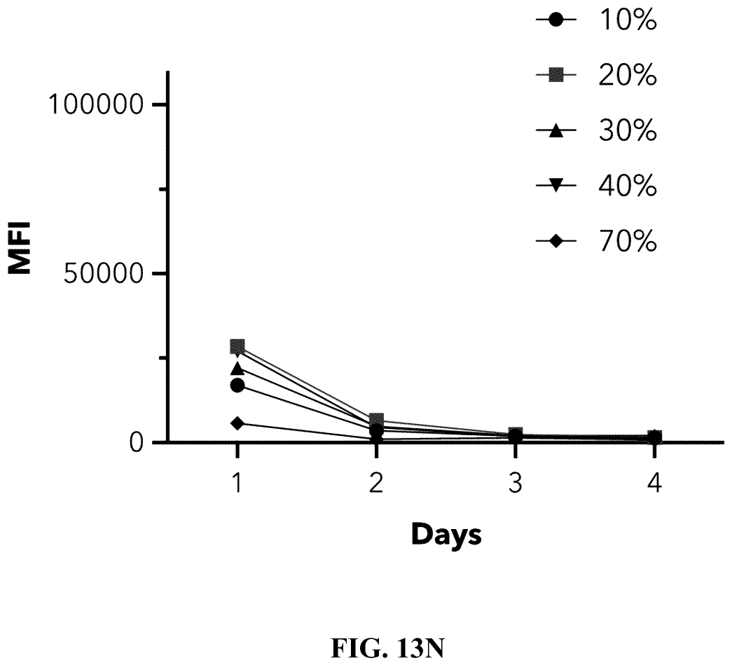

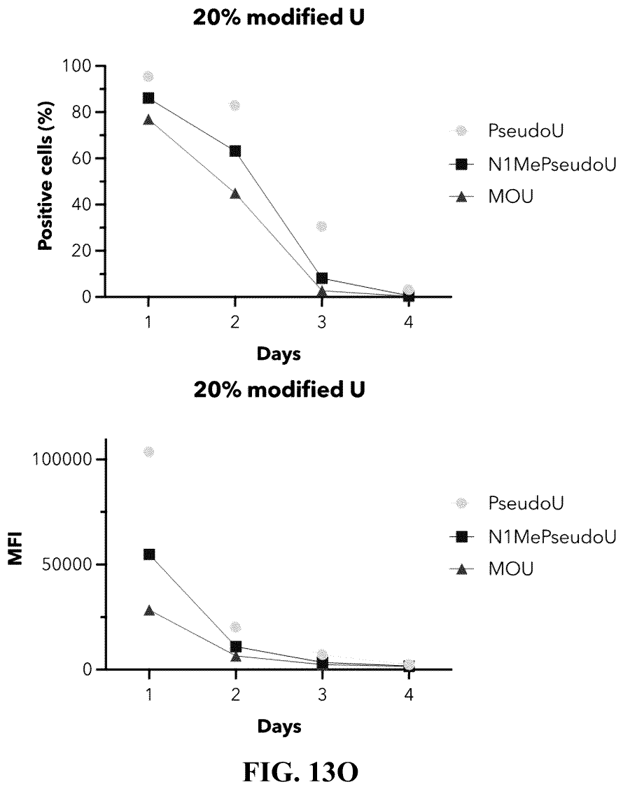

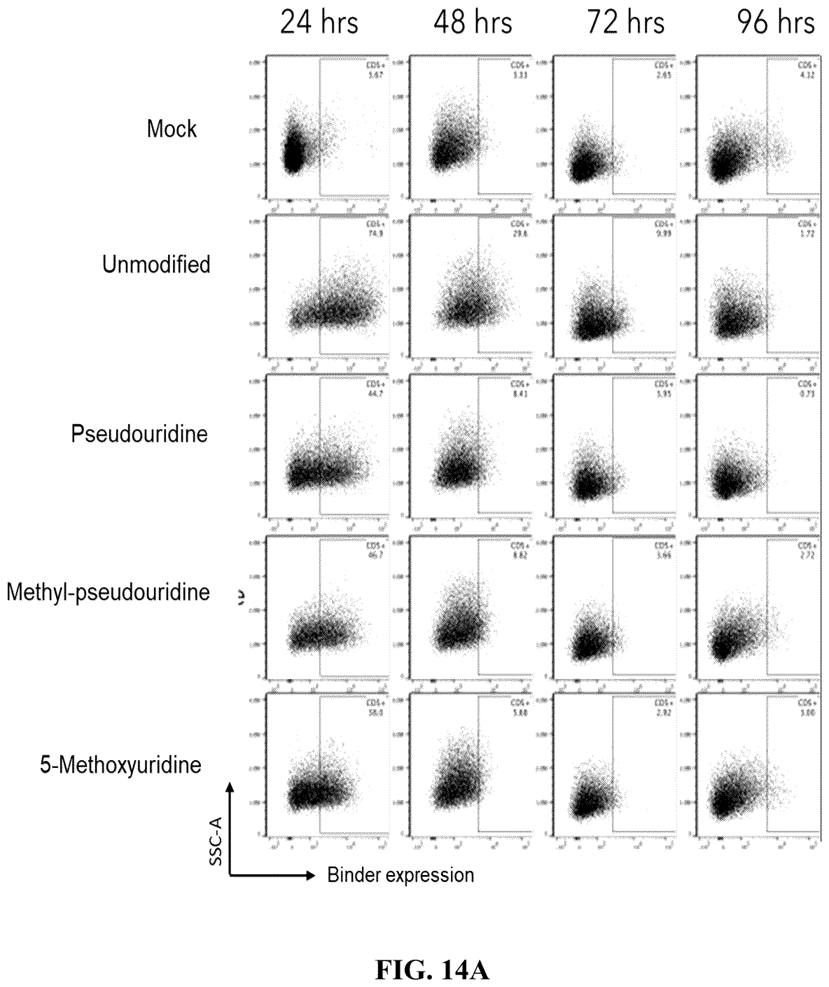

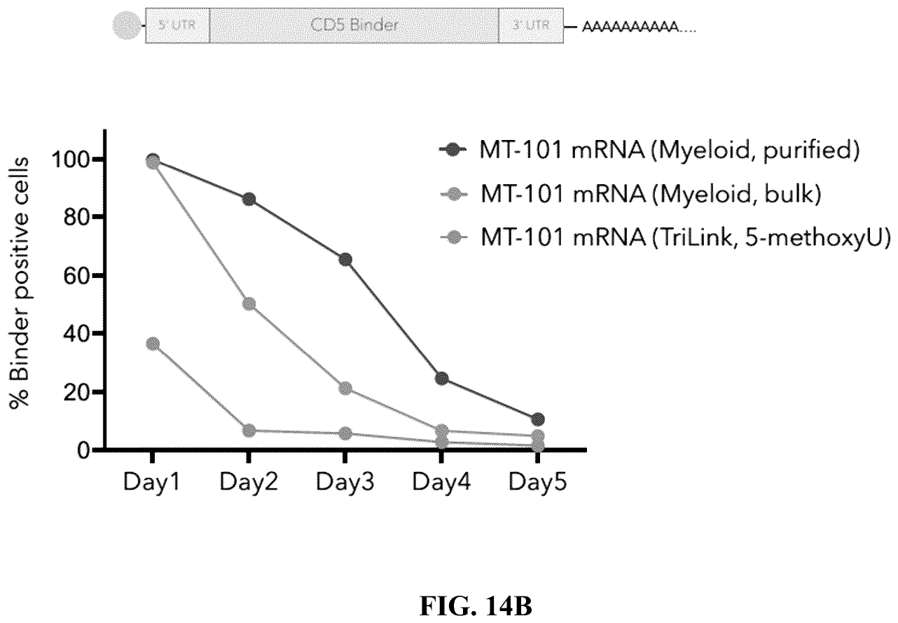

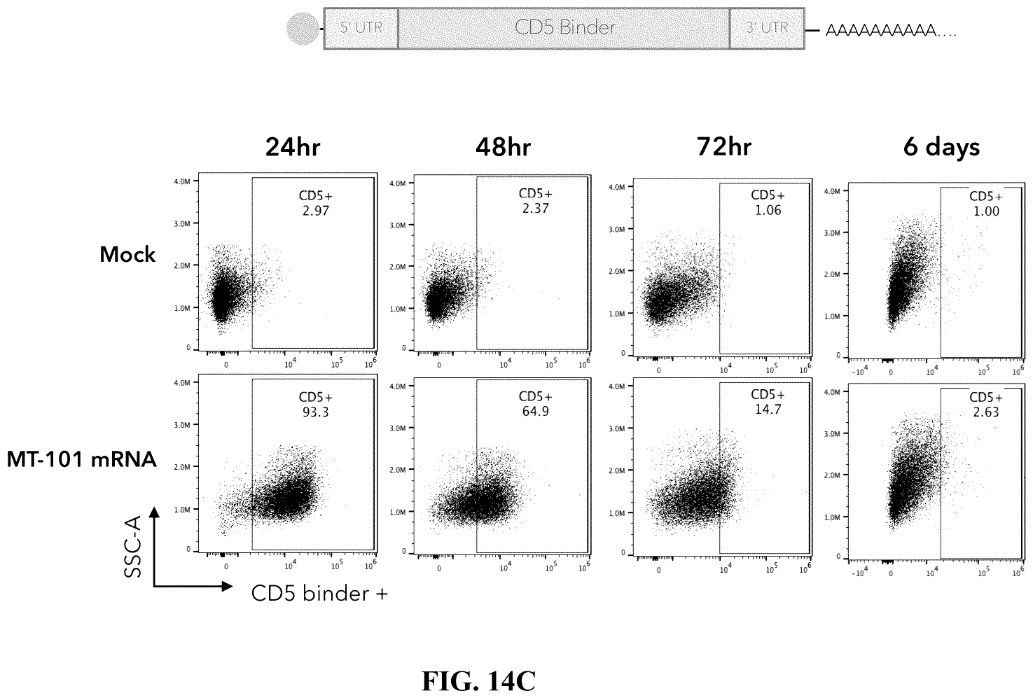

In this example, modified uridine nucleotides, such as pseudouridine (ψ), 1-methyl pseudouridine (me¹ψ), 5-methoxyuridine (5moU) (base structures illustrated in FIG. 13A) were tested for any effect on prolonging the half-life for mRNA expression in myeloid cells. However, results shown in FIG. 12 and FIG. 14A indicate that the complete modifications of all U residues in an mRNA had negligible effect on the CD5 CFP expression in THP-1 cells and primary human monocytes respectively. Further studies shown in FIG. 13B and FIGS. 13C-13O depict the effect of complete replacement of uridine bases with modified bases in an mRNA encoding HER2 CFP, compared to partial replacement of the U residues with the modified bases on the expression level of the encoded proteins. As shown in FIG. 13B, expression of the mRNA encoded protein was significantly lower in samples where uridine residues in the mRNA were completely modification to either pseudouridine, methyl-pseudouridine, or methoxy uridine; the partial modifications had remarkably enhanced expression levels of the encoded protein compared to the unmodified mRNA, with best results obtained when only 20% uridine bases in an mRNA sample were modified (FIGS. 13C-13O). In fact, as illustrated in FIG. 14B, in-house generated (myeloid) IVT-mRNA without U-modified nucleotides had better outcome on expression of the mRNA encoded protein than Trilink 5-moU modified mRNA. FIG. 14C shows enhanced expression of CD5 binders using the modified protocols described above.

### Example 16. Screening for UTRs

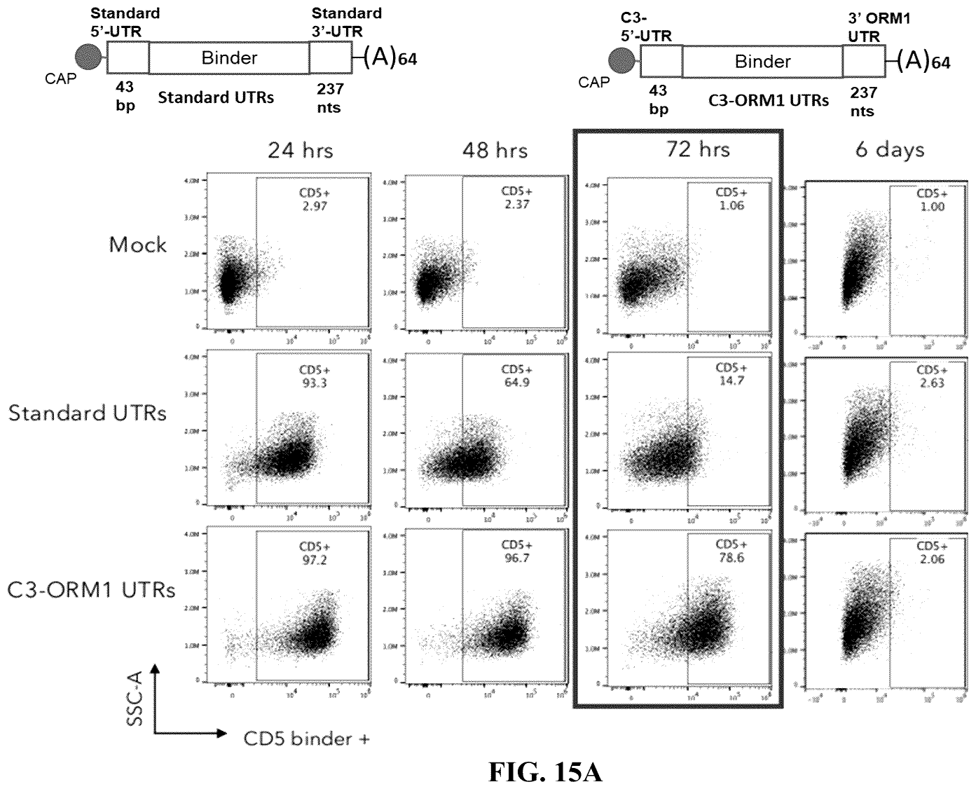

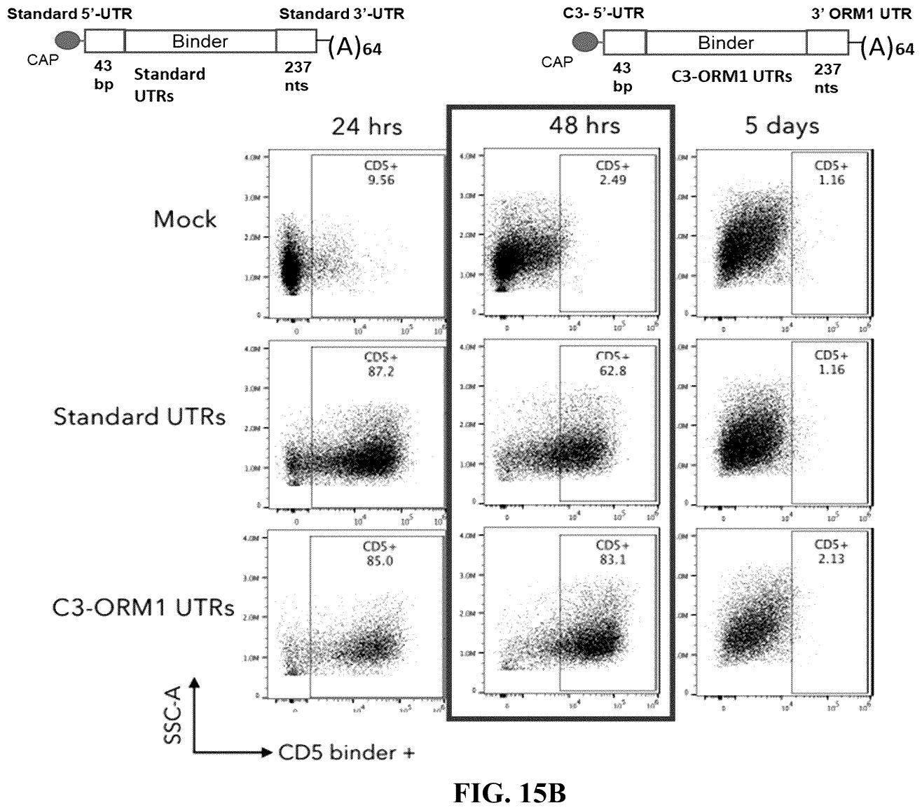

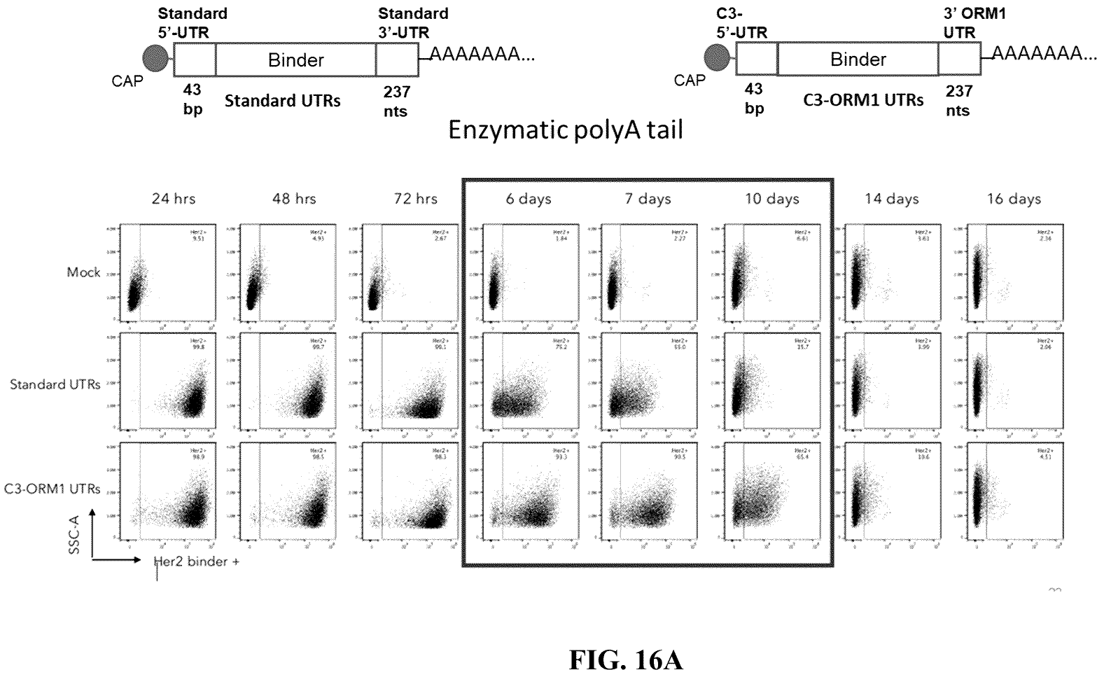

In this example, UTRs from different organisms were inserted flanking the mRNA coding sequence for the gene of interest to test the effect of various 5′-UTR: 3′-UTR sets on expression of the gene of interest. Various constructs having the respective 5′- and 3′-UTRs were prepared (see FIG. 1A and FIG. 1B) using the protocol shown in FIG. 2. Briefly, primary monocytes were frozen upon isolation from a healthy human donor. A healthy human donor, or donor in short is as understood in common language as being a human who has not been detected with a disease at the time the blood sample was drawn or is not convalescent or recovering from a disease or deficiency. Upon thawing, the cells were cultured for 1 hour, with TexMACs+MCSF in T 75 low binding flasks, harvested, EDTA washed to remove adherent cells, electroporated with 0.1 mg/ml RNA for 1×10\hat{ }8 cells/ml. Cells were then incubated in TexMACs+MCSF medium for 24 hours and tested for expression. A nucleic acid construct containing a C3 5′ UTR (presented herein as SEQ ID NO: 36 and as SEQ ID NO: 47) sequence and a ORM-1 3′ UTR (presented herein as SEQ ID NO: 43 and as SEQ ID NO: 53) sequence pair showed increased expression of the cargo mRNA sequence in myeloid cells at 24 hours. When tested at longer time points, remarkable effects were seen at 24, 48 and 72 hours, where the C3-ORM1 5′- and 3′-UTR pairs respectively were used compared to standard UTRs from different donors (FIGS. 15A and 15B). All constructs in this set had plasmid-encoded poly A tail 64 nucleotides long. Presence of these UTRs increased both the expression robustness, as well as the duration of expression of the binder construct. Even at 72 hours post electroporation, greater than 80% cells were showing expression of the binder CFP.

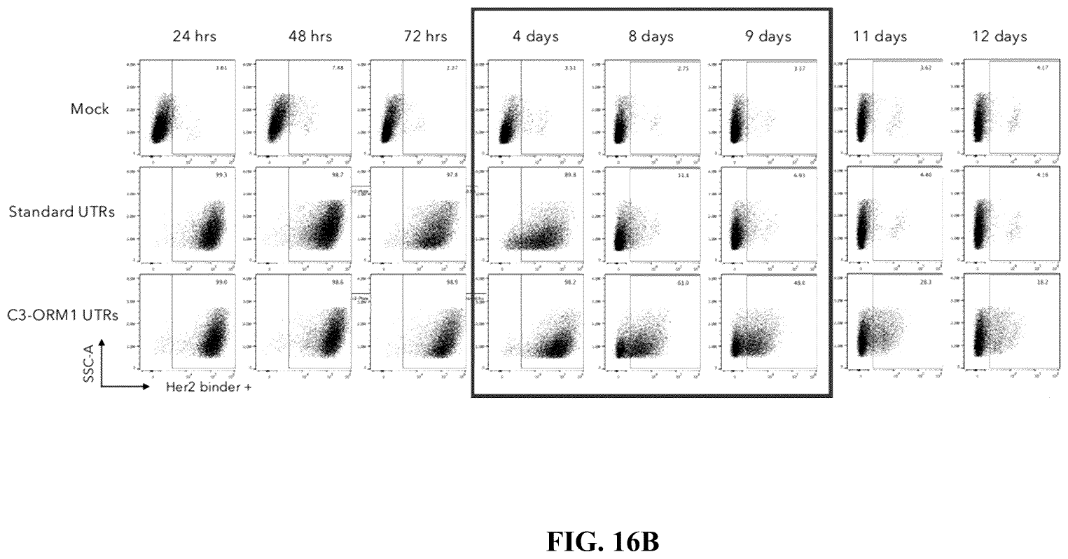

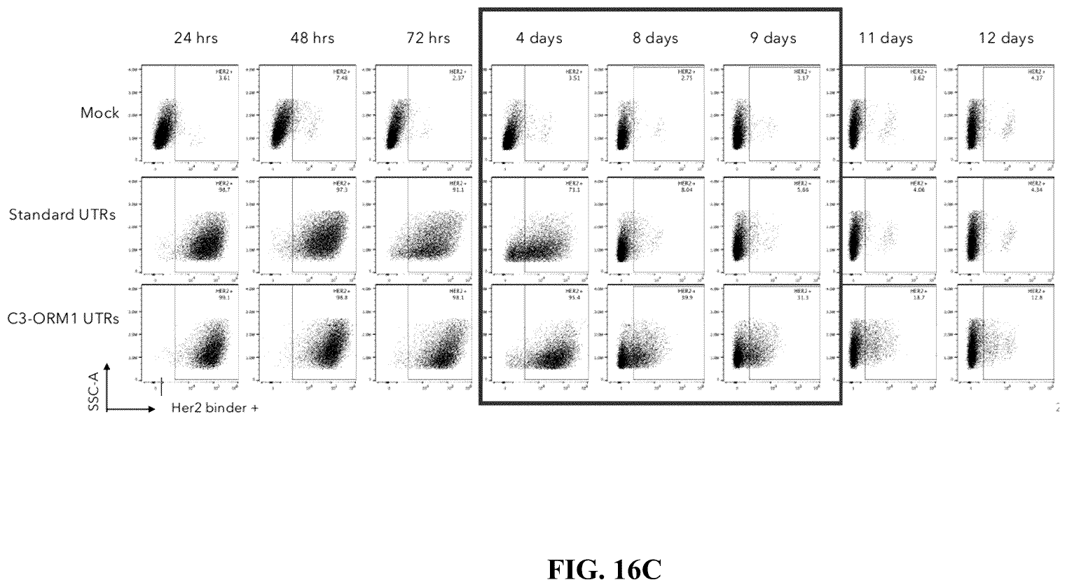

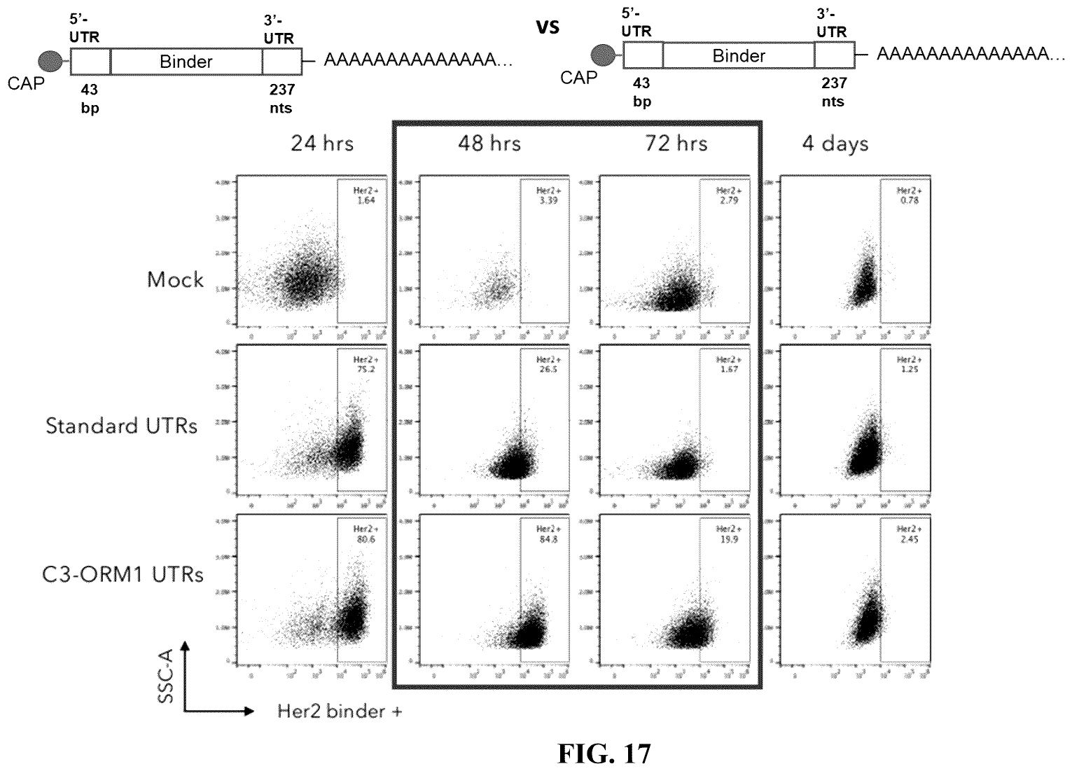

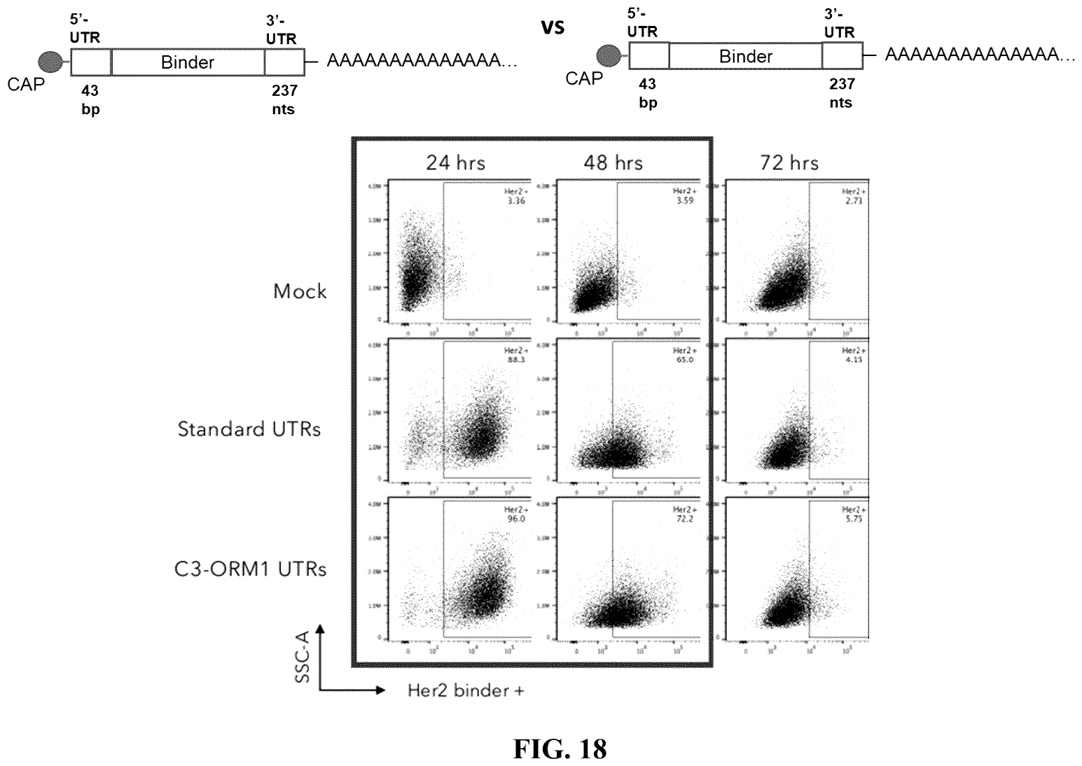

When enzymatic poly A tail was used instead of plasmid encoded poly A tail, (FIGS. 16A-16C) high and long-lasting expression levels of the encoded CFP were observed in THP-1 cells, with the expression being detectable at 10 days, and even at 14 days after electroporation. FIGS. 16A, 16B and 16C show that 100 micrograms/mL, 50 micrograms/mL, and as less as 25 micrograms/mL total RNA respectively were used to electroporate 2.5 million cells in the method as described above, and robust and long-lasting expression was evident in each case. The data were reproducible in monocyte samples from different human donors shown in FIGS. 17 and 18 respectively.

These results indicate that the combination of the UTRs and the poly A tail can lead to very high levels of expression of a candidate gene sequence in an mRNA construct, when expressed in a myeloid cell. These results have shown that the expressions were independent of the donor, or the gene of interest, as similar results were observed in both constructs encoding CD5-CFP and Her2-CFP.

1.-84. (canceled)

85. A composition comprising a recombinant mRNA comprising:
a sequence encoding a fused polypeptide, flanked by a non-native 5′ UTR sequence and a non-native 3′ UTR sequence,
wherein expression of the fused polypeptide encoded by the sequence of the recombinant mRNA is detected in a myeloid cell for at least 72 hours after introduction of the recombinant mRNA into the myeloid cell.

86. The composition of claim 85, wherein the recombinant mRNA comprises a 5′ methyl guanylate cap.

87. The composition of claim 85, wherein the 3′ UTR comprises a non-native poly A sequence, wherein the non-native poly A sequence is added enzymatically.

88. The composition of claim 87, wherein the non-native poly A sequence is about 50 to 250 nucleotides long.

89. The composition of claim 88, wherein the non-native poly A sequence is about 110 nucleotides long.

90. The composition of claim 85, wherein the expression of the fused polypeptide encoded by the sequence of the recombinant mRNA upon incorporating in the myeloid cell is at least 10% higher compared to a polypeptide encoded by a recombinant mRNA that (i) comprises a native 5′UTR and a non-native 3′UTR or (ii) lacks the non-native 5′ UTR sequence or the non-native 3′ UTR sequence.

91. The composition of claim 87, wherein the non-native 3′ UTR is enzymatically added to the recombinant mRNA and wherein the expression of the fused polypeptide encoded by the recombinant mRNA upon incorporating in the myeloid cell is at least 10% higher compared to a polypeptide encoded by a recombinant mRNA comprising a poly A sequence that is added non-enzymatically.

92. The composition of claim 85, wherein the non-native 5′UTR is at least 43 nucleotides in length.

93. The composition of claim 85, wherein the recombinant mRNA is an in vitro transcribed mRNA.

94. The composition of claim 86, wherein the +1 nucleotide of the 5′ methyl guanylate cap comprises a ribose methylated at the 2′O position.

95. The composition of claim 85, wherein less than 50% uridine residues of the recombinant mRNA are modified uridine residues; and wherein the modified uridine residue is a pseudouridine, 1-methyl-pseudouridine and/or a 5-methoxyuridine.

96. The composition of claim 85, wherein the recombinant mRNA comprises only unmodified residues.

97. The composition of claim 85, further comprising a lipid, wherein the lipid is one or more of a cationic lipid, a non-cationic lipid, and a PEGylated lipid.

98. The composition of claim 85, wherein incorporating comprises incorporating the recombinant mRNA in a myeloid cell in vivo or ex vivo.

99. The composition of claim 85, wherein the myeloid cell is a CD14+ cell, a CD14+CD16− cell, a CD14+CD16+ cell, a CD14−CD16+ cell, CD14−CD16− cell, a dendritic cell, an M0 macrophage, an M2 macrophage, an M1 macrophage or a mosaic myeloid cell/macrophage/dendritic cell.

100. The composition of claim 85, wherein the 5′ UTR sequence has at least 90% sequence identity to any one of SEQ ID NOs: 46-51; and/or the 3′ UTR sequence has at least 90% sequence identity to any one of SEQ ID NOs: 52-59.

101. A pharmaceutical composition comprising (I) the composition of claim 85, wherein the recombinant mRNA is isolated and purified mRNA and (II) a pharmaceutically acceptable excipient.

102. The pharmaceutical composition of claim 101, wherein the fused polypeptide is a chimeric fusion protein comprising an extracellular domain comprising an anti-CD5 binding domain or an anti-HER2 binding domain.

103. A method of treating a cancer in a subject in need thereof, the method comprising administering to a subject a therapeutically effective dose of the pharmaceutical composition of claim 101.

104. A method of expressing an exogenous polypeptide in a myeloid cell such that the expressed exogenous polypeptide is detectable for 72 hours or more, the method comprising:
(a) in vitro transcribing an mRNA comprising a sequence encoding the exogenous polypeptide, wherein the sequence encoding the exogenous polypeptide is flanked by a non-native 5′UTR and a non-native 3′UTR;
(b) enzymatically adding a poly A sequence to the 3′UTR; and
(c) incorporating the mRNA into the myeloid cell, and maintaining the myeloid cell in a microenvironment that endows cell survival and growth.

105. A composition comprising a recombinant mRNA comprising: a sequence encoding a fused polypeptide, flanked by a non-native 5′ UTR sequence and a non-native 3′ UTR sequence, wherein the 5′ UTR is at least 43 nucleotides in length.


SEQUENCE LISTING (69 sequences)

SEQ ID NO: 1 - protein, 116 aa, synthetic construct
  REGION 1..116: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 2 - protein, 107 aa, synthetic construct
  REGION 1..107: note = Description of Artificial Sequence: Synthetic polypeptide
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 3 - protein, 240 aa, synthetic construct
  REGION 1..240: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 4 - protein, 46 aa, synthetic construct
  REGION 1..46: note = Description of Artificial Sequence: Synthetic polypeptide
LYCRRLKIQVRKAAITSYEKSDGVYTGLSTRNQETYETLKHEKPPQ

SEQ ID NO: 5 - protein, 45 aa, synthetic construct
  REGION 1..45: note = Description of Artificial Sequence: Synthetic polypeptide
LYCRLKIQVRKAAITSYEKSDGVYTGLSTRNQETYETLKHEKPPQ

SEQ ID NO: 6 - protein, 42 aa, synthetic construct
  REGION 1..42: note = Description of Artificial Sequence: Synthetic polypeptide
RLKIQVRKAAITSYEKSDGVYTGLSTRNQETYETLKHEKPPQ

SEQ ID NO: 7 - protein, 42 aa, synthetic construct; identical to SEQ ID NO: 6
  REGION 1..42: note = Description of Artificial Sequence: Synthetic polypeptide

SEQ ID NO: 8 - protein, 35 aa, synthetic construct
  REGION 1..35: note = Description of Artificial Sequence: Synthetic polypeptide
YEDMRGILYAAPQLRSIRGQPGPNHEEDADSYENM

SEQ ID NO: 9 - protein, 21 aa, synthetic construct
  REGION 1..21: note = Description of Artificial Sequence: Synthetic peptide
IYIWAPLAGTCGVLLLSLVIT

SEQ ID NO: 10 - protein, 24 aa, synthetic construct
  REGION 1..24: note = Description of Artificial Sequence: Synthetic peptide
IYIWAPLAGTCGVLLLSLVITLYC

SEQ ID NO: 11 - protein, 62 aa, synthetic construct
  REGION 1..62: note = Description of Artificial Sequence: Synthetic polypeptide
ALSNSIMYFSHFVPVFLPAKPTTTPAPRPPTPAPTIASQPLSLRPEACRPAAGGAVHTRGLD

SEQ ID NO: 12 - protein, 130 aa, synthetic construct
  REGION 1..130: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 13 - protein, 108 aa, synthetic construct
  REGION 1..108: note = Description of Artificial Sequence: Synthetic polypeptide
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 14 - protein, 255 aa, synthetic construct
  REGION 1..255: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 15 - protein, 17 aa, synthetic construct
  REGION 1..17: note = Description of Artificial Sequence: Synthetic peptide
MWLQSLLLLGTVACSIS

SEQ ID NO: 16 - protein, 27 aa, synthetic construct
  REGION 1..27: note = Description of Artificial Sequence: Synthetic peptide
FWVLVVVGGVLACYSLLVTVAFIIFWV

SEQ ID NO: 17 - protein, 26 aa, synthetic construct
  REGION 1..26: note = Description of Artificial Sequence: Synthetic peptide
IYLIIGICGGGSLLMVFVALLVFYIT

SEQ ID NO: 18 - protein, 25 aa, synthetic construct
  REGION 1..25: note = Description of Artificial Sequence: Synthetic peptide
ILLPLIIGLILLGLLALVLIAFCII

SEQ ID NO: 19 - protein, 219 aa, synthetic construct
  REGION 1..219: note = Description of Artificial Sequence: Synthetic polypeptide
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 20 - protein, 171 aa, synthetic construct
  REGION 1..171: note = Description of Artificial Sequence: Synthetic polypeptide
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 21 - protein, 211 aa, synthetic construct
  REGION 1..211: note = Description of Artificial Sequence: Synthetic polypeptide
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 22 - protein, 86 aa, synthetic construct
  REGION 1..86: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVITLYC

SEQ ID NO: 23 - protein, 83 aa, synthetic construct
  REGION 1..83: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVIT

SEQ ID NO: 24 - protein, 432 aa, synthetic construct
  REGION 1..432: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 25 - protein, 436 aa, synthetic construct
  REGION 1..436: note = Description of Artificial Sequence: Synthetic polypeptide
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 26 - protein, 454 aa, synthetic construct
  REGION 1..454: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 27 - protein, 607 aa, synthetic construct
  REGION 1..607: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 28 - protein, 615 aa, synthetic construct
  REGION 1..615: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 29 - protein, 567 aa, synthetic construct
  REGION 1..567: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 30 - protein, 17 aa, synthetic construct
  REGION 1..17: note = Description of Artificial Sequence: Synthetic peptide
SSGGGGSGGGGSGGGGS

SEQ ID NO: 31 - protein, 7 aa, synthetic construct
  REGION 1..7: note = Description of Artificial Sequence: Synthetic peptide
SGGGGSG

SEQ ID NO: 32 - protein, 4 aa, synthetic construct
  REGION 1..4: note = Description of Artificial Sequence: Synthetic peptide
SGGG

SEQ ID NO: 33 - protein, 4 aa, synthetic construct
  REGION 1..4: note = Description of Artificial Sequence: Synthetic peptide
GSGS

SEQ ID NO: 34 - protein, 432 aa, synthetic construct; identical to SEQ ID NO: 24
  REGION 1..432: note = Description of Artificial Sequence: Synthetic polypeptide

SEQ ID NO: 35 - DNA, 17 bp, synthetic construct
  misc_feature 1..17: note = Description of Artificial Sequence: Synthetic oligonucleotide
  source 1..17: mol_type = other DNA
taatacgactcactata

SEQ ID NO: 36 - DNA, 64 bp, synthetic construct
  misc_feature 1..64: note = Description of Artificial Sequence: Synthetic oligonucleotide
  source 1..64: mol_type = other DNA
gggactcctccccatcctctccctctgtccctctgtccctctgaccctgcactgtcccagcacc

SEQ ID NO: 37 - DNA, 1299 bp, synthetic construct
  misc_feature 1..1299: note = Description of Artificial Sequence: Synthetic polynucleotide
  source 1..1299: mol_type = other DNA
atgtggctgcagtctctgctgctgctgggaacagtggcctgtagcatctctgagatccagctggttcagtctggcggcggacttgtgaaacctggcggatctgtcagaatcagctgtgccgccagcggctacaccttcaccaactacggcatgaactgggtccgacaggcccctggaaaaggccttgagtggatgggctggatcaatacccacaccggcgagccaacctacgccgatagctttaagggcagattcaccttcagcctggacgacagcaagaacaccgcctacctgcagatcaacagcctgagagccgaggataccgccgtgtacttctgcaccagaagaggctacgactggtacttcgatgtgtggggccagggcaccacagtgacagtttctagcggaggcggaggatcaggtggcggtggatctggcggtggtggctctgatatccagatgacacagagccctagcagcctgtctgcctctgtgggcgatagagtgaccatcacctgtagagccagccaggacatcaacagctacctgagctggttccagcagaagcctggcaaggcccctaagacactgatctaccgggccaacagactggaaagcggcgtgccaagcagattttctggcagcggctctggcaccgactacaccctgacaatcagcagcctgcagtacgaggacttcggcatctactactgccagcagtacgacgagagcccttggacatttggcggaggcaccaagctggaaatcaaatcaggcggcggaggaagcggagccctgagcaatagcatcatgtacttcagccacttcgtgcccgtgtttctgcccgccaagcctacaacaacacccgctcctagaccacctacaccagctcctacaatcgccagccagcctctgtctctcagacctgaagcctgtagacctgctgcaggcggagctgtgcataccagaggcctggatatctacatttgggcccctctggctggcacatgtggtgtcctgctgctgtctctggtcatcaccctgtactgcagacggctgaagatccaagtgcggaaggccgccatcaccagctacgagaaatctgatggcgtgtacaccggcctgagcacccggaatcaagagacatacgagacactgaagcacgagaagcctccacaaggcagcggcagctatgaggacatgagaggcattctgtacgccgctcctcagctgcggtctatcagaggccaacctggacctaaccacgaagaggacgccgactcctacgagaacatgtga

SEQ ID NO: 38 - DNA, 79 bp, synthetic construct
  misc_feature 1..79: note = Description of Artificial Sequence: Synthetic oligonucleotide
  source 1..79: mol_type = other DNA
atgagggccctgtgggtgctgggcctctgctgcgtcctgctgaccttcgggtcggtcagagctgacgatgaagttgatg

SEQ ID NO: 39 - DNA, 14 bp, synthetic construct
  misc_feature 1..14: note = Description of Artificial Sequence: Synthetic oligonucleotide
  source 1..14: mol_type = other DNA
tggccattggggcc

SEQ ID NO: 40 - DNA, 1653 bp, synthetic construct
  misc_feature 1..1653: note = Description of Artificial Sequence: Synthetic polynucleotide
  source 1..1653: mol_type = other DNA
atgatatccgtactgggtcccatttcggggcacgtgctgaaagccgtgtttagtcgcggcgatacgccggtgctgccgcacgagacgcgactcctgcagacgggtatccacgtacgcgtgagccagccctcgctgatcttggtatcgcagtacacgcccgactcgacgccatgccaccgcggcgacaatcagctgcaggtgcagcacacgtactttacgggcagcgaggtggagaacgtgtcggtcaacgtgcacaaccccacgggccgaagcatctgccccagccaggagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctgttgatgaacgggcagcagatcttcctggaggtacaagccatacgcgagaccgtggaactgcgtcagtacgatcccgtggctgcgctcttctttttcgatatcgacttgctgctgcagcgcgggcctcagtacagcgagcaccccaccttcaccagccagtatcgcatccagggcaagcttgagtaccgacacacctgggaccggcacgacgagggtgccgcccagggcgacgacgacgtctggaccagcggatcggactccgacgaagaactcgtaaccaccgagcgcaagacgccccgcgtcaccggcggcggcgccatggcgggcgcctccacttccgcgggccgcaaacgcaaatcagcatcctcggcgacggcgtgcacgtcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggaattcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggt

SEQ ID NO: 41 - DNA, 13 bp, synthetic construct
  misc_feature 1..13: note = Description of Artificial Sequence: Synthetic oligonucleotide
  source 1..13: mol_type = other DNA
ggccttgttggcc

SEQ ID NO: 42 - DNA, 107 bp, synthetic construct
  misc_feature 1..107: note = Description of Artificial Sequence: Synthetic polynucleotide
  source 1..107: mol_type = other DNA
tgatccccatcgctgtgggtggtgccctggcggggctggtcctcatcgtcctcatcgcctacctcgtcggcaggaagaggagtcacgcaggctaccagactatctag

SEQ ID NO: 43 - DNA, 118 bp, synthetic construct
  misc_feature 1..118: note = Description of Artificial Sequence: Synthetic polynucleotide
  source 1..118: mol_type = other DNA
caggacacagccttggatcaggacagagacttgggggccatcctgcccctccaacccgacatgtgtacctcagctttttccctcacttgcatcaataaagcttctgtgtttggaacag

SEQ ID NO: 44 - protein, 436 aa, synthetic construct; identical to SEQ ID NO: 25
  REGION 1..436: note = Description of Artificial Sequence: Synthetic polypeptide

SEQ ID NO: 45 - protein, 454 aa, synthetic construct; identical to SEQ ID NO: 26
  REGION 1..454: note = Description of Artificial Sequence: Synthetic polypeptide

SEQ ID NO: 46 - RNA, 47 nt, synthetic construct
  misc_feature 1..47: note = Description of Artificial Sequence: Synthetic oligonucleotide
  source 1..47: mol_type = other RNA
gggaaataagagagaaaagaagagtaagaagaaatataagagccacc

SEQ ID NO: 47 - RNA, 61 nt, synthetic construct
  misc_feature 1..61: note = Description of Artificial Sequence: Synthetic oligonucleotide
  source 1..61: mol_type = other RNA
actcctccccatcctctccctctgtccctctgtccctctgaccctgcactgtcccagcacc

SEQ ID NO: 48 - RNA, 50 nt, synthetic construct
  misc_feature 1..50: note = Description of Artificial Sequence: Synthetic oligonucleotide
  source 1..50: mol_type = other RNA
acatttgcttctgacacaactgtgttcactagcaacctcaaacagacacc

SEQ ID NO: 49 - DNA, 90 bp, synthetic construct
  misc_feature 1..90: note = Description of Artificial Sequence: Synthetic oligonucleotide
  source 1..90: mol_type = other DNA
cttgcccgccgatatctctgccgggtgactagctgcttcctttctctctcgcgcgcggtgtggtggcagcaggcgcagcccagcctcgaa

SEQ ID NO: 50 - DNA, 63 bp, synthetic construct
  misc_feature 1..63: note = Description of Artificial Sequence: Synthetic oligonucleotide
  source 1..63: mol_type = other DNA
cttcctttttgtccgacatcttgacgaggctgcggtgtctgctgctattctccgagcttcgca

SEQ ID NO: 51 - DNA, 58 bp, synthetic construct
  misc_feature 1..58: note = Description of Artificial Sequence: Synthetic oligonucleotide
  source 1..58: mol_type = other DNA
agcaatcctttctttcagctggagtgctcctcaggagccagccccacccttagaaaag

SEQ ID NO: 52 - RNA, 110 nt, synthetic construct
  misc_feature 1..110: note = Description of Artificial Sequence: Synthetic polynucleotide
  source 1..110: mol_type = other RNA
tgataataggctggagcctcggtggccatgcttcttgccccttgggcctccccccagcccctcctccccttcctgcacccgtacccccgtggtctttgaataaagtctga

SEQ ID NO: 53 - RNA, 118 nt, synthetic construct
  misc_feature 1..118: note = Description of Artificial Sequence: Synthetic polynucleotide
  source 1..118: mol_type = other RNA
caggacacagccttggatcaggacagagacttgggggccatcctgcccctccaacccgacatgtgtacctcagctttttccctcacttgcatcaataaagcttctgtgtttggaacag

SEQ ID NO: 54 - RNA, 114 nt, synthetic construct
  misc_feature 1..114: note = Description of Artificial Sequence: Synthetic polynucleotide
  source 1..114: mol_type = other RNA
gctcgctttcttgctgtccaatttctattaaaggttcctttgttccctaagtccaactactaaactgggggatattatgaagggccttgagcatctggattctgcctaataaaa

SEQ ID NO: 55 - RNA, 228 nt, synthetic construct
  misc_feature 1..228: note = Description of Artificial Sequence: Synthetic polynucleotide
  source 1..228: mol_type = other RNA
gctcgctttcttgctgtccaatttctattaaaggttcctttgttccctaagtccaactactaaactgggggatattatgaagggccttgagcatctggattctgcctaataaaagctcgctttcttgctgtccaatttctattaaaggttcctttgttccctaagtccaactactaaactgggggatattatgaagggccttgagcatctggattctgcctaataaaa

SEQ ID NO: 56 - DNA, 53 bp, synthetic construct
  misc_feature 1..53: note = Description of Artificial Sequence: Synthetic oligonucleotide
  source 1..53: mol_type = other DNA
ctggagagaatcacagatgtggaatatttgtcataaataaataatgaaaacct

SEQ ID NO: 57 - DNA, 51 bp, synthetic construct
  misc_feature 1..51: note = Description of Artificial Sequence: Synthetic oligonucleotide
  source 1..51: mol_type = other DNA
ataggtccaaccagctgtacatttggaaaaataaaactttattaaatcaaa

SEQ ID NO: 58 - DNA, 278 bp, synthetic construct
  misc_feature 1..278: note = Description of Artificial Sequence: Synthetic polynucleotide
  source 1..278: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 59 - RNA, 243 nt, synthetic construct
  misc_feature 1..243: note = Description of Artificial Sequence: Synthetic polynucleotide
  source 1..243: mol_type = other RNA
gctgaagaagtgggaatgggagcactctgtcttctttgctagagaagtggagagaaaatacaaaaggtaaagcagttgagattctctacaacctaaaaaattcctaggtgctattttcttatcctttgtactgtagctaaatgtacctgagacatattagtctttgaaaaataaagttatgtaaggttttttttatctttaaatagctctgtgggttttaacatttttataaagatataccaa

SEQ ID NO: 60 - RNA, 13 nt, synthetic construct
  misc_feature 1..13: note = Description of Artificial Sequence: Synthetic oligonucleotide
  source 1..13: mol_type = other RNA
wwwtatttatttw

SEQ ID NO: 61 - protein, 62 aa, synthetic construct
  REGION 1..62: note = Description of Artificial Sequence: Synthetic polypeptide
KKVAKKPTNKAPHPKQEPQEINFPDDLPGSNTAAPVQETLHGCQPVTQEDGKESRISVQERQ

SEQ ID NO: 62 - protein, 9 aa, unidentified
  REGION 1..9: note = Description of Unknown: TRP2 epitope sequence
SVYDFFVWL

SEQ ID NO: 63 - protein, 25 aa, synthetic construct
  REGION 1..25: note = Description of Artificial Sequence: Synthetic peptide
GWVKPIIIGHHAYGDQYRATDFVVP

SEQ ID NO: 64 - RNA, 64 nt, synthetic construct
  misc_feature 1..64: note = Description of Artificial Sequence: Synthetic oligonucleotide
  source 1..64: mol_type = other RNA
aaaaaaaaaaaaaaaaaaaaaaaaaaaaaaaaaaaaaaaaaaaaaaaaaaaaaaaaaaaaaaaa

SEQ ID NO: 65 - RNA, 74 nt, synthetic construct
  misc_feature 1..74: note = Description of Artificial Sequence: Synthetic oligonucleotide
  source 1..74: mol_type = other RNA
aaaaaaaaaaaaaaaaaaaaaaaaaaaaaaaaaaaaaaaaaaaaaaaaaaaaaaaaaaaaaaaaaaactagtgc

SEQ ID NO: 66 - RNA, 153 nt, synthetic construct
  misc_feature 1..153: note = Description of Artificial Sequence: Synthetic polynucleotide
  misc_feature 4..153: note = SITE - This sequence may encompass 100-150 residues
  source 1..153: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - RNA, 14 nt, synthetic construct
  misc_feature 1..14: note = Description of Artificial Sequence: Synthetic oligonucleotide
  source 1..14: mol_type = other RNA
aaaaaaaaaaaaaa

SEQ ID NO: 68 - RNA, 10 nt, synthetic construct
  misc_feature 1..10: note = Description of Artificial Sequence: Synthetic oligonucleotide
  source 1..10: mol_type = other RNA
aaaaaaaaaa

SEQ ID NO: 69 - RNA, 200 nt, synthetic construct
  misc_feature 1..200: note = Description of Artificial Sequence: Synthetic polynucleotide
  misc_feature 1..200: note = SITE - This sequence may encompass 50-200 nucleotides
  source 1..200: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa